(19) United States
(12) Patent Application Publication
(10) Pub. No.: US 2022/0362357 A1
(43) Pub. Date: Nov. 17, 2022

(54) Synergistic Combinations of Amino Acid Depletion Agent Sensitizers (AADAS) and Amino Acid Depletion Agents (AADA), and Therapeutic Methods of Use Thereof

(71) Applicant: STICHTING RADBOUD UNIVERSITAIR MEDISCH CENTRUM, NIJMEGEN (NL)

(72) Inventors: Franciscus Nicolaas VAN LEEUWEN, BILTHOVEN (NL); Laurens VAN DER MEER, BILTHOVEN (NL)

(21) Appl. No.: 17/271,824

(22) PCT Filed: Dec. 6, 2018
(86) PCT No.: PCT/EP2018/083893

§ 371 Date: Feb. 26, 2021

Related U.S. Application Data
(60) Provisional application No. 62/725,313, filed on Aug. 31, 2018.

(51) Int. Cl. A61K 38/50; A61K 38/51; A61K 31/519; A61K 31/4985; A61K 31/52; A61K 31/506; A61K 31/336; A61K 31/505; A61K 31/517; A61K 31/4545; A61K 35/18; A61P 35/00

(52) U.S. Cl. CPC A61K 38/50; A61K 38/51; A61K 31/519; A61K 31/4985; A61K 31/52; A61P 35/00; A61K 31/336; A61K 31/505; A61K 31/517; A61K 31/4545; A61K 35/18; A61K 31/506

(57) ABSTRACT

Disclosed herein are synergistically effective combinations of Amino Acid Depletion Agents (AADA) and Amino Acid Depletion Agent Sensitizers (AADAS). Also disclosed are methods of using the disclosed combinations to treat subjects with a disease treatable by amino acid depletion-induced cell death (e.g. apoptosis). For example, the disclosed combinations are useful in the treatment or the manufacture of a medicament for use in the treatment of adult and pediatric cancers, in particular, acute lymphoblastic leukemia (ALL), as well as other conditions where amino acid depletion-induced apoptosis is expected to have a therapeutically useful effect. The synergistic combinations are also effective against solid tumors and lymphomas, including gastric cancer, pancreatic cancer, NK lymphoma, DLBCL, colorectal cancer, bladder cancer, hepatic cancer and glioblastoma.

### CROSS-REFERENCE TO RELATED APPLICATIONS

This application claims priority to U.S. Provisional Application No. 62/725,313, filed on 31 Aug. 2018, and incorporated herein by reference in its entirety.

### FIELD OF THE INVENTION

The invention relates to the treatment of cancers using combinations of amino acid depletion agents (AADA) and amino acid depletion agent-sensitizers (AADAS), which render cancer cells more susceptible to AADA-induced cell death (e.g. apoptosis).

### SUMMARY OF THE INVENTION

Asparaginase (ASNase) is a key component in the treatment of acute lymphoblastic leukemia (ALL), and is under clinical evaluation for other malignancies. A poor response to ASNase is associated with increased relapse risk. Commercially available ASNases are of bacterial origin, and their primary therapeutic effect is to deplete Asn from blood plasma. Approved versions include native E. coli asparaginase (ELSPAR, Lundbeck Inc.), an E. coli-derived peg-conjugated ASNase (e.g. ONCASPAR®, Servier) and an E. chrysanthemi ASNase (ERWINAZE®, EUSA Pharma). As some tumor cells, including leukemic blasts, are selectively dependent on the exogenous supply of Asn, systemic administration of ASNase leads to a cessation of growth and the induction of cell death. In ALL, the response of leukemic cells to ASNase (i.e. cellular quiescence or some degree of apoptosis) is dependent on the genetic makeup of the leukemia and the extent of Asn depletion in the cellular microenvironment.

Unfortunately, leukemic cells present in the bone marrow niche or the central nervous system (CNS) appear to be less responsive to ASNase treatment as a result of incomplete Asn depletion, enhancing the chance of relapse. Another clinical problem with the use of this protein drug is the formation of inhibitory antibodies over time that cause silent inactivation of the drug. A phenomenon associated with this immune response is the occurrence of severe allergies and related toxicities. Once patients have developed an allergic reaction to the drug, treatment must be stopped, which enhances the chances of a relapse and reduces the chance of cure once a relapse has developed.

To address these challenges, some groups have been exploring the benefits of conjugating polymers to ASNases, to make (for example) new PEGylated and PASylated forms of ASNase. One company has addressed these challenges by encapsulating the ASNase within red blood cells (see Patents U.S. Pat. No. 8,974,802 & U.S. Pat. No. 8,617,840, both to Erytech Pharma SA), thereby enabling ASNase activity to be safely circulated in a patient's blood stream for extended periods of time. Another group has attempted to reduce the toxicity of ASNase by eliminating its Glutaminase (GLNase) activity (see WO 2018/050918, to Cambridge Innovations Technologies and WO 2017/151707, to the University of Illinois). And yet another group has developed endotoxin-free ASNases (WO 2018/085493 to Georgia State Research Foundation), having improved safety/toxicity profiles.

Despite these innovations, there remains a long-felt need to improve the efficacy of amino acid depletion therapies, particularly in cases where amino acid depletion alone is insufficient to completely cure or place the disease or condition into remission.

To address this problem, the Applicants performed an in vitro loss-of-function screen to identify novel therapeutic interventions that may synergize with ASNase to induce cell death (e.g. apoptosis) rather than mere cell quiescence. The results of this screen indicated that specifically interfering with a cell's ability to cope with amino acid starvation enhances the clinical efficacy of amino acid depletion agents (AADA).

Blocking cellular stress responses at certain kinases (e.g. the amino acid sensor GCN2, a key mediator of the amino acid stress response), sensitized cells to AADA-induced apoptosis. Unexpectedly, the Applicants observed that blocking Bruton's Tyrosine Kinase (BTK) was at least as effective in sensitizing cells to AADA-induced apoptosis. Moreover, the GCN2 kinase was effectively inhibited in response to inhibition of BTK activity, demonstrating that BTK regulates the activity of this branch of the amino acid stress response. In contrast, and rather unexpectedly, inhibition of mTOR, another important component of the amino acid stress response route, and known effector of BTK, did not affect response to AADA-induced apoptosis.

Accordingly, a first object of the disclosure is to provide therapeutically effective combinations of amino acid depletion agent sensitizers (AADAS) and amino acid depletion agents (AADA) for the treatment of diseases, including cancers. In some embodiments, the AADAS is a Bruton's Tyrosine Kinase inhibitor (BTKi) and the AADA is an ASNase. Now that the disclosure has been made, the skilled artisan will reasonably expect that a safe and effective amount of any BTK inhibitor will sensitize a variety of different tumor cells to ASNase-induced apoptosis. As further disclosed below, the efficacy of the combination of the BTKi and ASNase is greater than the additive efficacy of either component by itself. Applicants envision that other combinations of AADAS and AADA will likewise yield synergistic efficacy against cells from various cancer types.

In some embodiments, the therapeutically effective combinations provide synergistic efficacy against one or more cancers as compared with the efficacy of either active alone.

In other embodiments, the synergistic combinations are therapeutically effective against cancer types that are non-responsive to one or both of the AADA and the AADAS.

In some particular embodiments, the AADA is an ASNase and the AADAS is a BTKi, each present in subtherapeutic amounts. As used herein, a “subtherapeutic amount” means an amount of a drug or therapeutic agent that is ineffective at producing or eliciting a given therapeutic effect (e.g. a significant reduction in the size of a tumor, a significant decrease in the number of tumor cells or a significant decrease in the metastatic potential of tumor cells).

Accordingly, synergistic combinations according to this disclosure may exhibit at least the following patterns of synergistic efficacy against a given cancer indication and/or cell type:
a) AADA Efficacy of 1+AADAS Efficacy of 1=Combination Efficacy of 3, 4 or greater;
b) AADA Efficacy of 1+AADAS Efficacy of 0=Combination Efficacy of 2, 3, 4 or greater;
c) AADA Efficacy of 0+AADAS Efficacy of 1=Combination Efficacy of 2, 3, 4 or greater;
d) AADA Efficacy of 0+AADAS Efficacy of 0=Combination Efficacy of 1, 2, 3, 4 or greater;

In a second object, the disclosure provides methods of treating diseases including cancers comprising sequential or simultaneous administration of synergistically effective combinations of AADA and AADAS as disclosed herein.

In a third object, the disclosure provides kits comprising effective amounts of an AADA and an AADAS, optionally including instructions for use thereof in treating cancers.

In a fourth object, the disclosure provides methods of manufacture of a medicament comprising effective amounts of an AADA and an AADAS.

In a fifth object, the disclosure provides methods and/or uses of combinations of AADA and AADAS in the treatment of cancer. In some embodiments, the use is effective in inducing tumor cells that are resistant to treatment with either the AADA or the AADAS alone. In some embodiments, the use of the combination of AADA and AADAS is effective in treating a patient in whom a cancer has relapsed after a treatment with either the AADA or AADAS previously administered as a monotherapy, or in combination with an agent other than the AADA (in the case where the AADAS was previously administered) or the AADAS (in the case where the AADA was previously administered).

In a sixth object, the disclosure provides methods and/or uses of combinations of AADA and AADAS in the treatment of cancer that is resistant to either or both of the AADA or the AADAS, when administered alone or with an agent other than the corresponding AADA or AADAS. In some embodiments, simultaneous or sequential administration of individually subtherapeutic doses of the AADA and AADAS restores the sensitivity of the tumor cells. In some embodiments, the entire population of tumor cells is killed by a combination of the AADA and AADAS, but not either the AADA or AADAS alone.

It is a further object of the invention to not encompass within the invention any previously known product, process of making the product, or method of using the product such that the Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product.

These and other embodiments are disclosed or are obvious from and encompassed by, the following Detailed Description.

### BRIEF DESCRIPTION OF THE DRAWINGS

FIG. 1A is a diagram outlining the Dox inducible Cas9 Kinome gRNA library;

FIG. 1B is a WB image showing Dox regulation of Cas9 expression;

FIG. 1C is a graph showing the abundance of sgRNAs before and after treatment;

FIG. 1D is a graph showing the genes whose deletions correlate most significantly with resistance (right) or sensitization (left) to ASNase treatment;

FIG. 2A depicts several components of the signaling pathways modulated by treatment of cells with ASNase. KO of TRIB3 correlates with resistance to ASNase, while KO of p90RSK, EEF2K or GCN2 correlates with sensitivity to ASNase;

FIG. 2B is an image of a WB indicating TRIB3 levels in deletion pools of cells representing two independent gRNAs (2.1-3, 2.2-3)

FIG. 2C is an image of a WB indicting GCN2 levels in deletion pools of cells representing two independent gRNAs (1.1, 1.2)

FIG. 2D is a graph showing the growth of TRIB3 deletion cells versus control cells;

FIG. 2E is a graph showing deletion of TRIB3 protects cells from ASNase-induced death;

FIG. 2F is a graph showing deletion of TRIB3 reduces the percent of SubG1 cells in response to ASNase treatment;

FIG. 2G is a graph showing the relative viability among control, TRB3del_2.1-3 and TRIB3del_2.2-3;

FIG. 2H is a graph showing that deletion of GCN2 increases the percent of SubG1 cells in response to ASNase treatment;

FIG. 2I is a graph showing the relative viability among control, GCN2KO-1.1 and GCN2KOdel_1.2;

FIG. 3A depicts several components signaling pathways downstream of B cell receptor signaling, including BTK;

FIG. 3B is a schematic representation of the in vitro ASNase treatment of nalm6 BTK KO cells vs. control nalm6 cells;

FIG. 3C is a WB image showing the relative levels of BTK in the indicated control and selected BTK KO clones, obtained after single cell cloning;

FIG. 3D is a WB image showing the relative levels of cleaved and uncleaved PARP in the indicated control and selected BTK KO clones;

FIG. 3E is a graph of percent dead cells in control or BTK KO clones, plus and minus 5 IU/mL ASNase;

FIG. 3F is a graph showing percent dead cells in control or GCN2KO clones, plus and minus 1 IU/mL ASNase;

FIG. 3G are graphs showing Hoechst staining for control and BTK KO clones, either untreated, treated with 5 IU/mL ASNase, or washed out after treatment with 5 IU/mL ASNase;

FIG. 3H is a graph of the percent of cells in SubG1 in control or BTK KO clones, either untreated, treated with 5 IU/mL ASNase (non-washout experiment);

FIG. 4A is a schematic representation of the in vitro ASNase+ibrutinib treatment of nalm6 cells, followed by a colony assay after washout of ASNase and Ibrutinib;

FIG. 4B is a WB showing the effective inhibition of BTK via loss of phospho-BTK with increasing concentration of ibrutinib;

FIG. 4C are graphs showing Hoechst staining for untreated cells and cells treated with 1 IU/mL ASNase, plus 0, 1 or 10 μM ibrutinib;

FIG. 4D is a graph showing the percent of cells in SubG1 for Sem, Nalm6 and Reh cells treated with 0 or 1 IU/mL ASNase and 0, 1 or 10 μM ibrutinib;

FIG. 4E is a WB image and a graph showing PARP cleavage for Nalm6, Sem and Reh cells treated with 0 or 1 IU/mL ASNase and 0 or 10 μM ibrutinib;

FIG. 4F is a graph showing the percent SubG1 cells for Sem, Nalm6 and Reh cells treated with 0 or 1 IU/mL ASNase and 0, 1 or 10 μM ibrutinib;

FIG. 4G are images of plates seeded with Nalm6 cells treated with 0 or 1 IU/mL ASNase plus 0, 1 or 10 μM ibrutinib;

FIG. 4H are graphs presenting the colony counts for Nalm6 cells and Sem cells (below);

FIG. 4I is a schematic indicating that effective amounts of both ASNase+ibrutinib induce apoptosis, whereas these same amounts of either active alone induce quiescence;

FIG. 5A presents a schematic overview representing the workflow used to derive drug synergies in ALL cell lines and patient-derived xenograft (PDX) samples;

FIG. 5B is an example heat map showing the synergistic killing efficacy of the combination of ASNase and ibrutinib;

FIG. 5C is a schematic indicating that synergistic killing efficacy was observed in a large majority of PDX samples treated with ASNase and ibrutinib;

FIG. 5D is a graph showing the relative viability for a selection of PDX cells subjected to the indicated concentration of ASNase plus or minus the indicated concentrations of ibrutinib;

FIG. 5E is a graph showing the percent 7AAD positive cells for the indicated PDX samples treated with the indicated concentrations of ASNase and/or ibrutinib;

FIG. 5F is a summary table indicating the synergistic killing efficacy of the combination of ASNase and ibrutinib against patient derived xenografts (PDX);

FIG. 5G is a summary table indicating the synergistic killing efficacy of the combination of ASNase and ibrutinib (CI calculated without the 100 μM ibrutinib group);

FIG. 6A are graphs showing the Hoechst staining for Nalm6 cells treated with 0 or 5 μg/mL prednisone plus 0, 1 or 10 μM ibrutinib;

FIG. 6B are WB images showing the cleavage of PARP Nalm6 cells treated with 0 or 5 μg/mL prednisone plus 0, 1 or 10 μM ibrutinib;

FIG. 6C are graphs showing the percent of SubG1 cells for Nalm6, Sem and 697 cells treated with 0 or 5 μg/mL prednisone plus 0, 1 or 10 μM ibrutinib;

FIG. 7A is a schematic showing the experimental design for the transplantation of patient-derived xenografts (PDX) into immunocompromised mice. Briefly, mice were injected intrafemorally with 500,000 leukemic blasts, then two weeks after transplantation, mice were administered the indicated treatment for 9 consecutive days;

FIG. 7B is a graph showing the bodyweight of the mice during and after treatment;

FIG. 7C is a graph showing the percent of hCD45+ cells post-transplant;

FIG. 7D is a graph showing the percent of hCD10+ cells post-transplant;

FIG. 7E is graph showing the percent hCD19+ cells as a function of treatment;

FIG. 7F is a Kaplan-Meier plot showing event-free survival. Significance was tested using a log-rank test;

FIG. 8A is a diagram presenting how inhibition of BTK blocks amino acid depletion agent (AADA) activation of the GCN2-ATF4 axis;

FIG. 8B is a series of WB images showing the impact on GCN2, ATF4 and ASNS protein expression in Nalm6 or Sem cells treated according to the following: untreated, ibrutinib, ASNase or combination of ibrutinib and ASNase;

FIG. 9 is a heat map presenting the results of the reverse phase proteomics analysis (top 50 normalized against NALM6 & SEM controls) performed on NA;

FIG. 10A is a graph showing the relative viability for HBP-ALL cells subjected to the indicated concentrations of ASNase and 0, 0.1, 1 or 10 μM Ibrutinib;

FIG. 10B is a graph of percent dead HBP-ALL cells treated with either 0 or 0.001 IU/mL ASNase and 1 or 10 μM Ibrutinib (3 day treatment);

FIG. 11A is a graph showing the relative viability for LOUCY cells subjected to the indicated concentrations of ASNase and 0, 0.1, 1 or 10 μM Ibrutinib;

FIG. 11B is a graph of percent dead LOUCY cells treated with either 0 or 0.0001 IU/mL ASNase and 1 or 10 μM Ibrutinib (3 day treatment);

FIG. 12A is a graph showing the relative viability for SupT1 cells subjected to the indicated concentrations of ASNase and 0, 0.1, 1 or 10 μM Ibrutinib;

FIG. 12B is a graph of percent dead SupT1 cells treated with either 0 or 1 IU/mL ASNase and 1 or 10 μM Ibrutinib (3 day treatment);

FIG. 12C is a graph of percent dead SupT1 cells treated with either 0 or 1 IU/mL ASNase and 1 or 10 μM Ibrutinib (7 day treatment);

FIG. 13A is a graph showing the relative viability for SupT1 cells subjected to the indicated concentrations of ASNase and 0, 0.1, 1 or 10 μM Ibrutinib;

FIG. 13B is a graph of percent dead SupT1 cells treated with either 0 or 1 IU/mL ASNase and 1 or 10 μM Ibrutinib (7 day treatment);

FIG. 14A is a graph showing percent viability for refractory patient-derived ALL cells subjected to the indicated concentrations of ASNase and 0, 1 or 10 μM Ibrutinib;

FIG. 14B is a graph showing the AUC for the ASNase treatment shown in FIG. 14A;

FIG. 15A is a graph showing % cell viability for SU-DHL-10 (DLBCL) cells subjected to 0.0032, 0.016, 0.08, 0.4, 2 or 10 U/mL L-ASNase and 0.0032, 0.016, 0.08, 0.4, 2 or 10 μM Ibrutinib for 4 days of treatment;

FIG. 15B is a Combination Index (CI) plot indicating that the combination of ASNase and Ibrutinib exerted synergistic killing efficacy against SU-DHL-10 cells;

FIG. 15C is the histogram plot for the SU-DHL-10 study;

FIG. 16 is a graph showing % cell viability for NCI-N87 (Gastric cancer) cells subjected to 0.0032, 0.016, 0.08, 0.4, 2 or 10 U/mL L-ASNase and 0.0032, 0.016, 0.08, 0.4, 2 or 10 μM Ibrutinib for 4 days of treatment. Analysis indicated synergistic efficacy;

FIG. 17 is a graph showing % cell viability for AsPC-1 (Pancreatic cancer) cells subjected to 0.0032, 0.016, 0.08, 0.4, 2 or 10 U/mL L-ASNase and 0.0032, 0.016, 0.08, 0.4, 2 or 10 μM Ibrutinib for 4 days of treatment. Analysis indicated synergistic efficacy;

FIG. 18 is a graph showing % cell viability for CAPAN-1 (Pancreatic cancer) cells subjected to 0.0032, 0.016, 0.08, 0.4, 2 or 10 U/mL L-ASNase and 0.0032, 0.016, 0.08, 0.4, 2 or 10 μM Ibrutinib for 4 days of treatment. Analysis indicated synergistic efficacy;

FIG. 19 is a graph showing % cell viability for BxPC-3 (Pancreatic cancer) cells subjected to 0.0032, 0.016, 0.08, 0.4, 2 or 10 U/mL L-ASNase and 0.0032, 0.016, 0.08, 0.4, 2 or 10 μM Ibrutinib for 4 days of treatment. Analysis indicated synergistic efficacy;

FIG. 20 is a graph showing % cell viability for KHYG-1 (NK Lymphoma cancer) cells subjected to 0.0032, 0.016, 0.08, 0.4, 2 or 10 U/mL L-ASNase and 0.0032, 0.016, 0.08, 0.4, 2 or 10 μM Ibrutinib for 4 days of treatment. Analysis indicated synergistic efficacy;

FIG. 21 is a graph showing % cell viability for HT-29 (Colorectal cancer) cells subjected to 0.0032, 0.016, 0.08, 0.4, 2 or 10 U/mL L-ASNase and 0.0032, 0.016, 0.08, 0.4, 2 or 10 μM Ibrutinib for 4 days of treatment. Analysis indicated synergistic efficacy;

FIG. 22 is a graph showing % cell viability for RT4 (Bladder cancer) cells subjected to 0.0032, 0.016, 0.08, 0.4, 2 or 10 U/mL L-ASNase and 0.0032, 0.016, 0.08, 0.4, 2 or 10 μM Ibrutinib for 4 days of treatment. Analysis indicated synergistic efficacy;

FIG. 23 is a graph showing % cell viability for Hep3B (Hepatic cancer) cells subjected to 0.0032, 0.016, 0.08, 0.4, 2 or 10 U/mL L-ASNase and 0.0032, 0.016, 0.08, 0.4, 2 or 10 μM Ibrutinib for 4 days of treatment. Analysis indicated synergistic efficacy;

### DETAILED DESCRIPTION OF THE INVENTION

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

The term “about,” as used herein, means approximately, in the region of, roughly, or around. When the term “about” is used in conjunction with a numerical range, it modifies that range by extending the boundaries above and below the recited values. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Asparaginase (ASNase) is a key component of the multi-drug treatment that is used to treat pediatric leukemia. Upon administration, this protein converts the amino acid asparagine into aspartate, effectively depleting the blood of asparagine. In contrast to most cells, lymphocytes and leukemic blasts cannot produce asparagine in sufficient amounts. The limited availability of asparagine activates the amino acid stress response pathway, which induces a state of cellular quiescence, allowing cells to overcome periods of nutrient starvation. That said, sustained amino acid starvation will eventually induce apoptosis via this same pathway.

However, the success of ASNase therapy is compromised by protection of tumor cells by the cellular microenvironment, causing tumor cells to go into a quiescent cell survival mode, rather than to induce apoptosis. To address this problem, Applicants performed a CRISPR/Cas9-based in vitro loss-of-function screen to identify novel therapeutic interventions that may synergize with ASNase to induce apoptosis rather than cell quiescence (see Wang et al. Science 2014; 343 (6166):80-4). Briefly, the screen was performed using NALM6 pre-B ALL cells exposed to a IC₅₀ dose of ASNase. To facilitate rapid translation into clinical practice, the screen focused on kinases as potential targets for pharmacological intervention. After treatment, genomic DNA from treated and untreated control cells was isolated, and incorporated gRNA sequences were amplified and sequenced (Illumina HISEQT™) to identify gRNAs that were selectively enriched or depleted during treatment. The results were validated using targeted knockouts and by using small-molecule inhibitors.

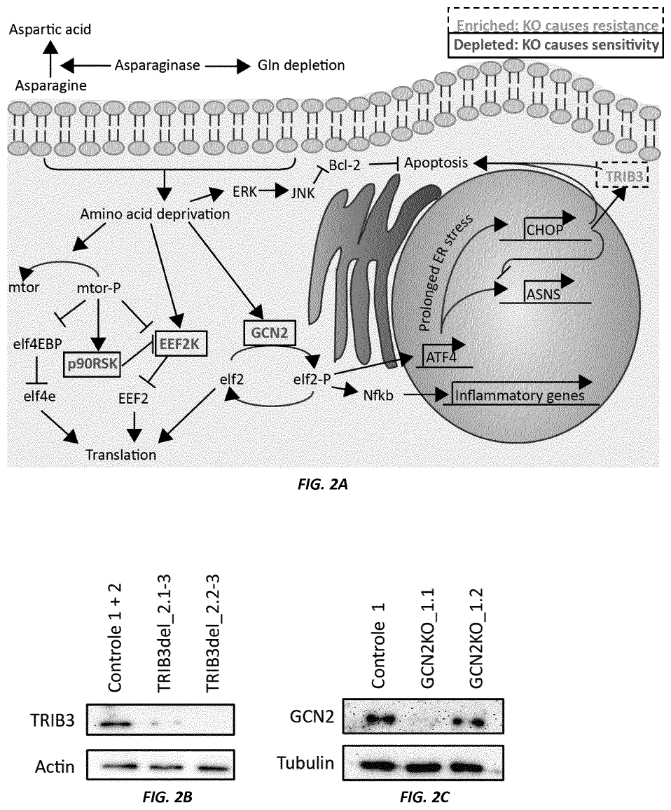

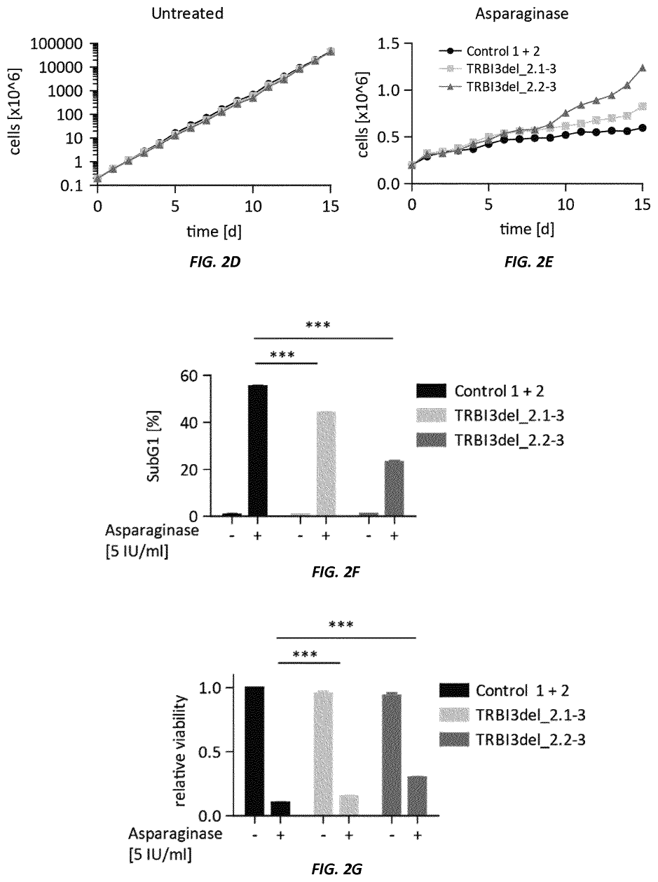

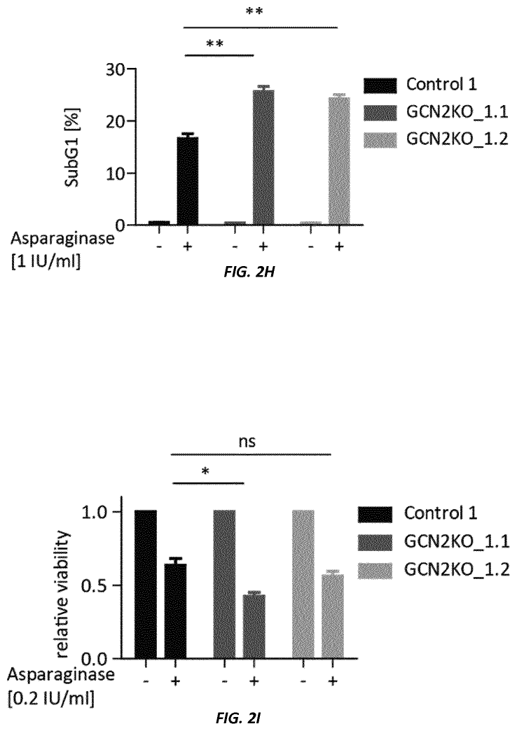

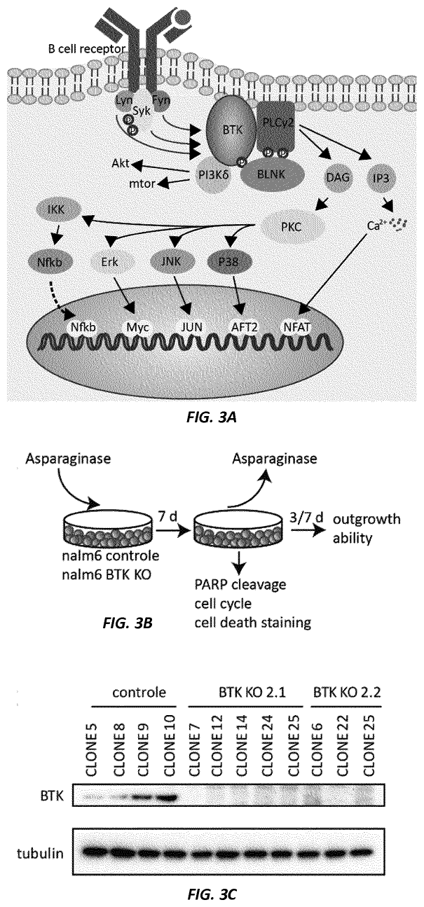

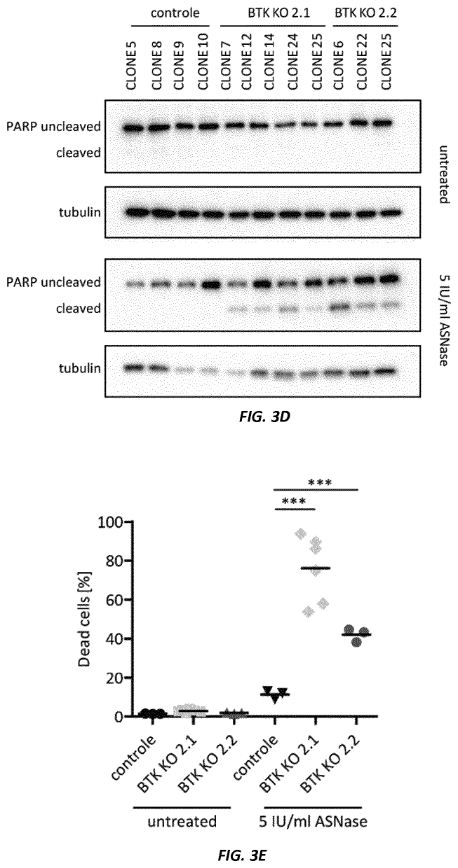

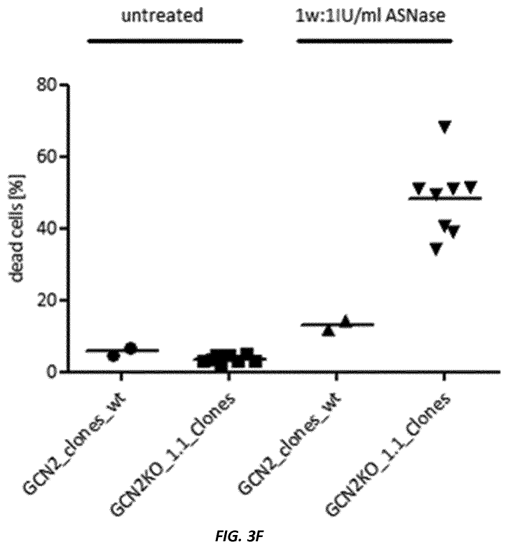

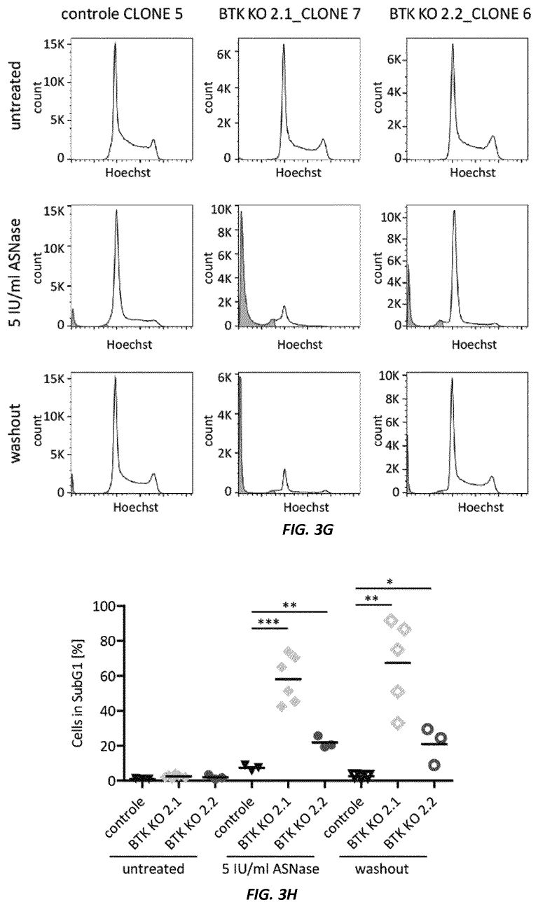

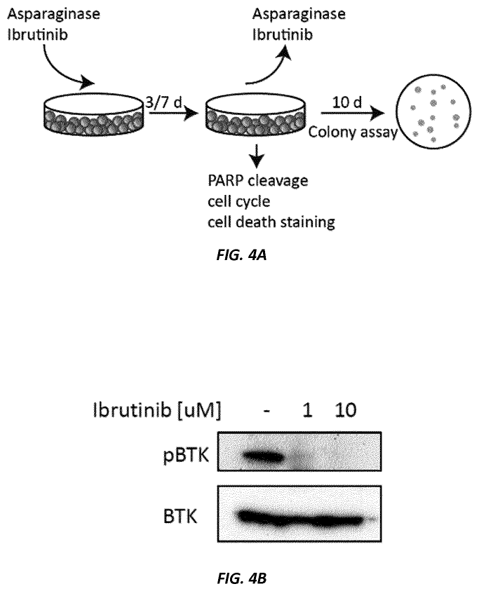

As detailed in the Examples below, candidate genes were validated using ALL cell lines and a co-culture of hTERT immortalized MSCs and ALL-xenografts. FIG. 2A depicts several elements of the amino acid deprivation signaling pathway, which is modulated by treatment with ASNase. FIGS. 3A & 3B summarize genes whose deletion promotes either resistance or sensitization to treatment of the cells with the AADA ASNase.

As disclosed herein, the anti-tumor effects of ASNase impinge on changes in cell metabolism that occur as a result of amino acid starvation. Consistent with this notion, the loss of function screen identified genes either directly involved in the amino acid response route (TRIB3) or inhibition of protein translation in response to amino acid starvation (GCN2). Indeed, knockout of GCN2 sensitized cells to ASNase treatment whereas depletion of TRIB3 was sufficient to render these cells more resistant to the effects of ASNase on cell growth. Accordingly, the Applicants have identified novel pathway interactions that can potentiate or resist the apoptosis inducing effects of the amino acid depletion agent (AADA) ASNase.

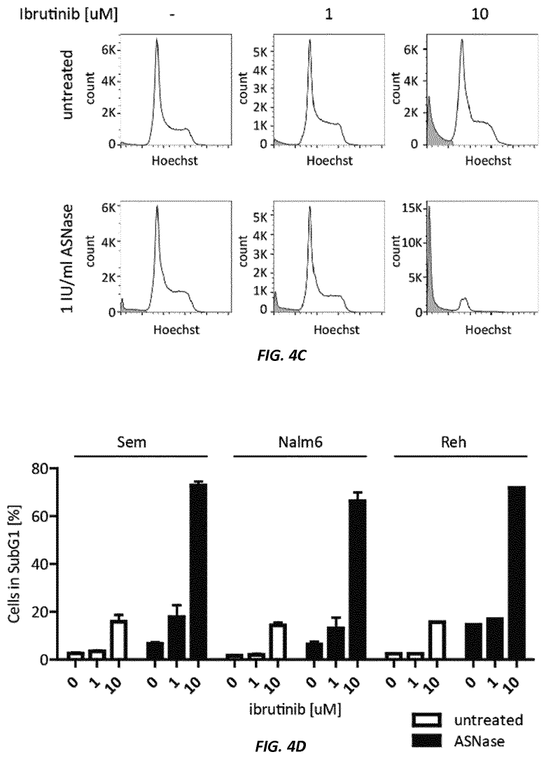

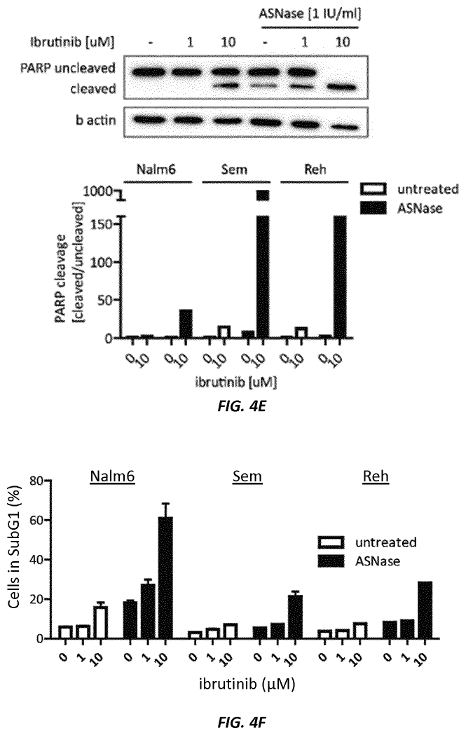

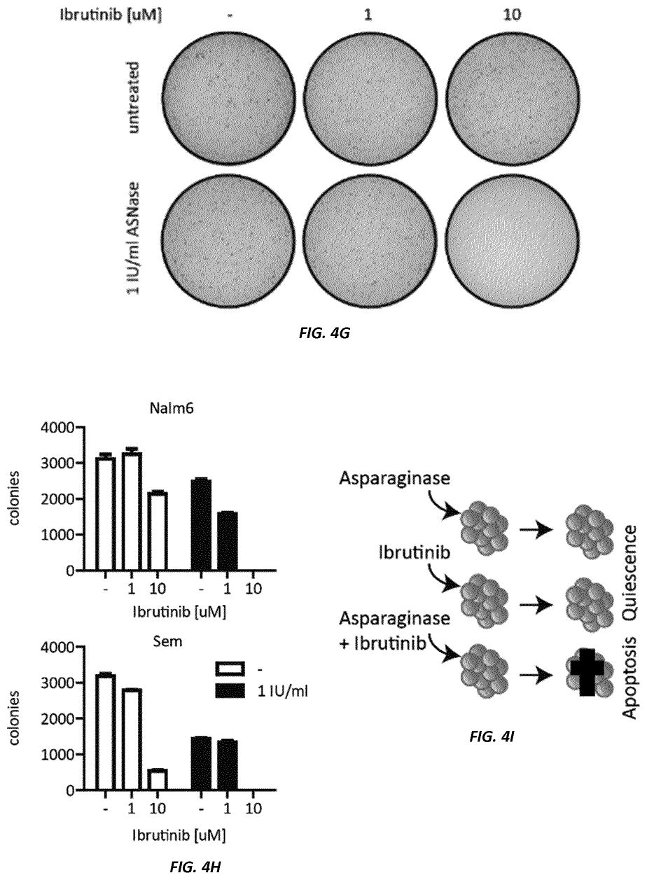

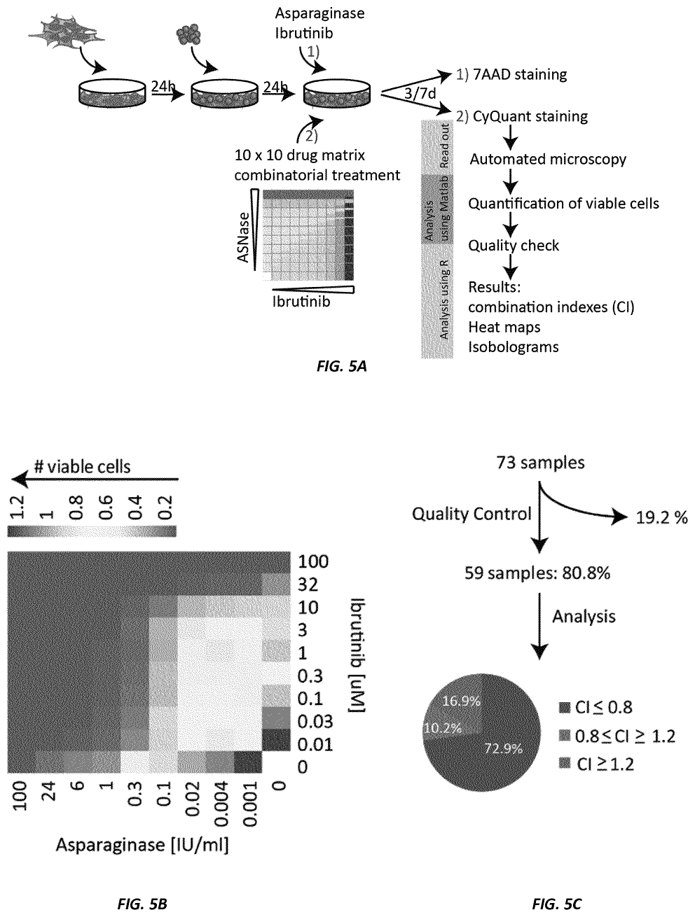

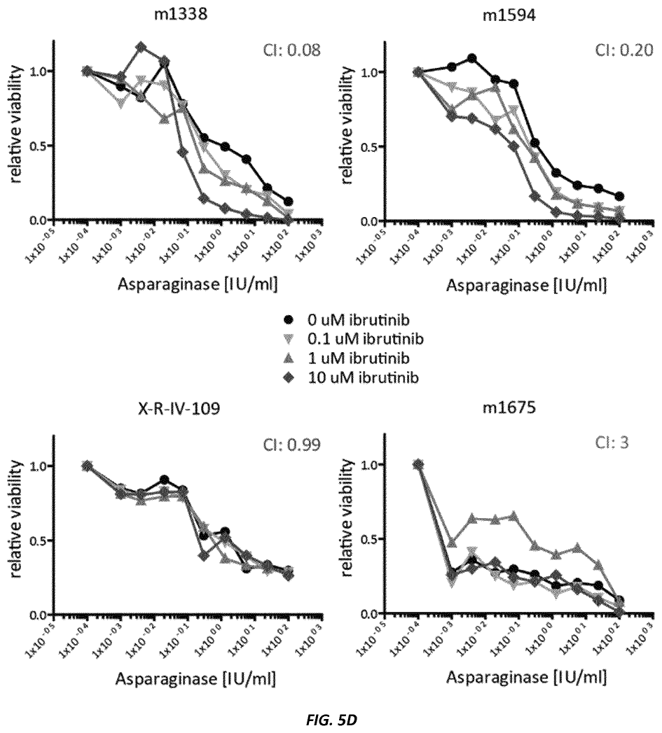

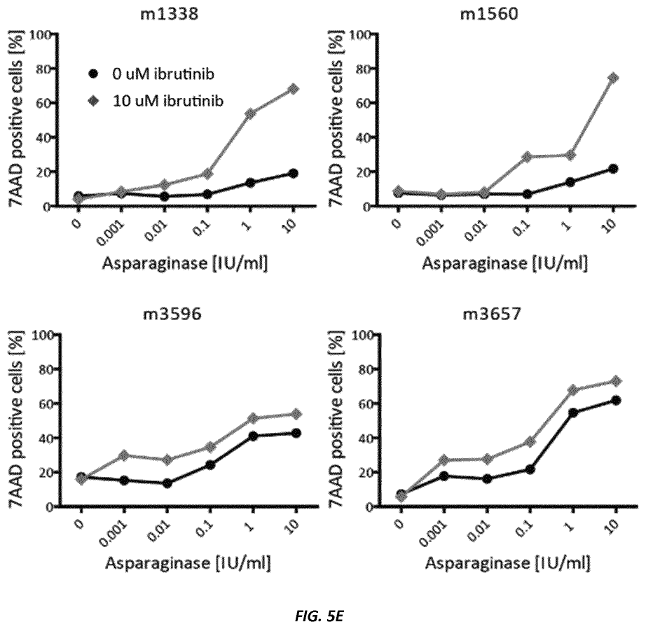

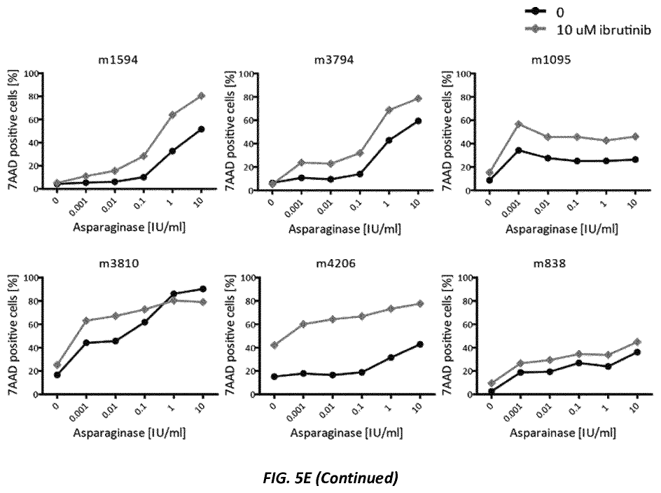

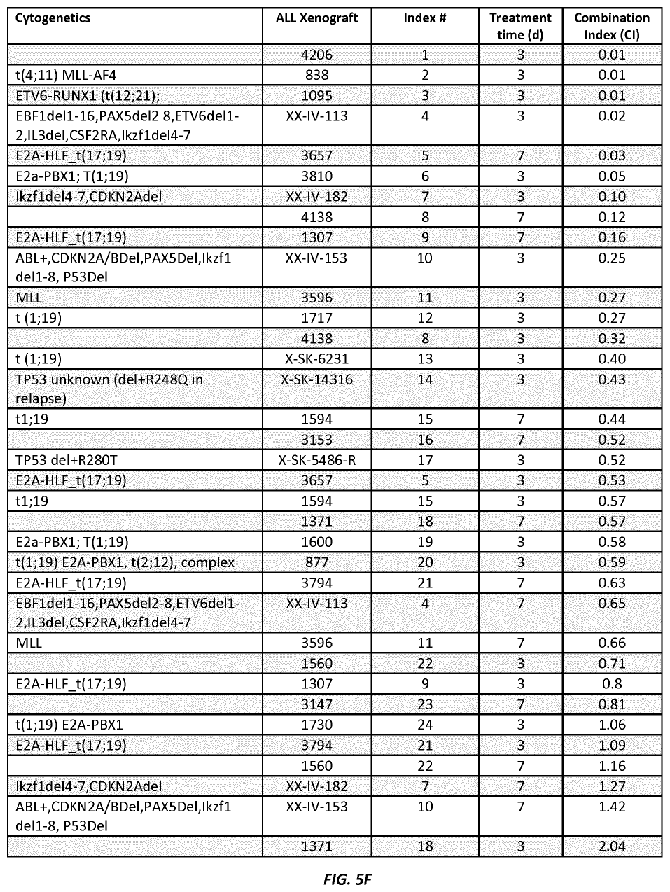

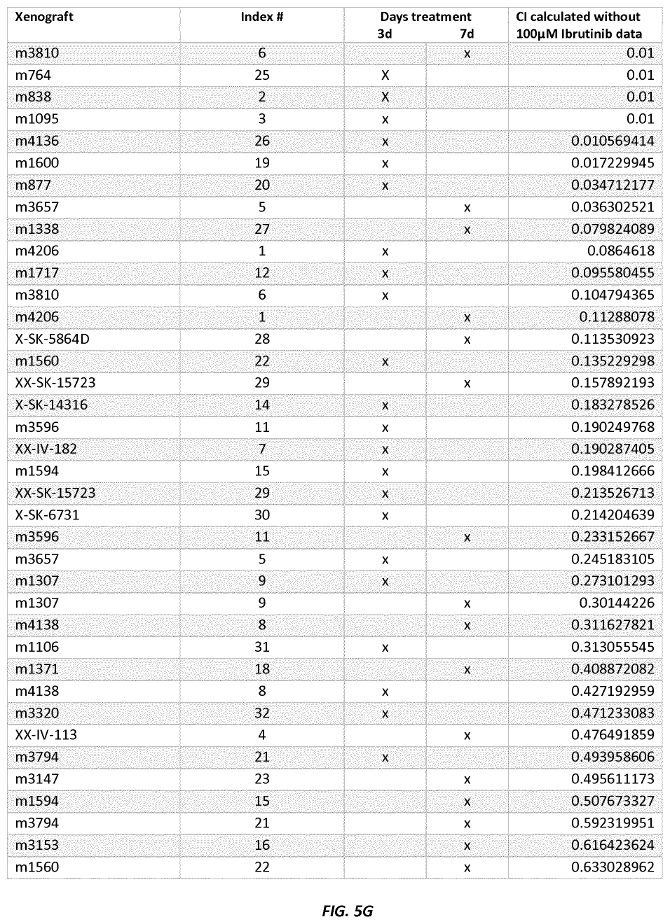

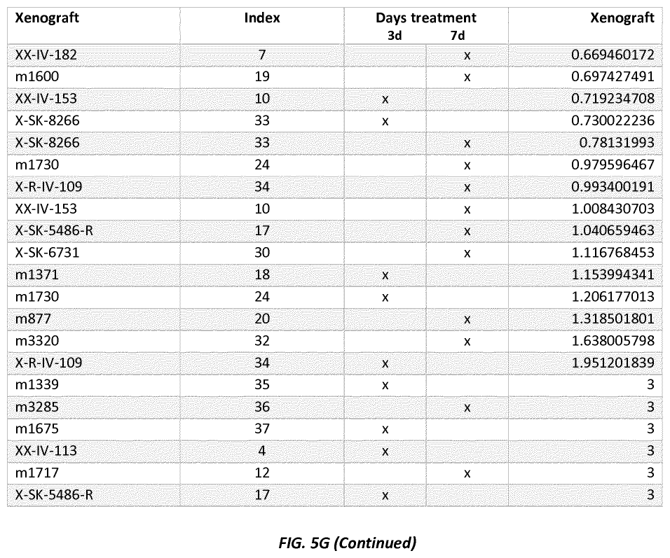

As further disclosed herein, Bruton's Tyrosine Kinase (BTK), a hematopoietic-cell specific protein kinase acting downstream of the B-cell receptor, was demonstrated to protect ALL cells from ASNase-induced apoptosis. Indeed, targeted knockout as well as inhibition by the FDA-approved BTK inhibitor ibrutinib, strongly enhanced ASNase-induced apoptosis in a variety of ALL cell lines (see e.g. FIGS. 4B & 4D). Moreover, Applicants tested the effect of combinations of ASNase and ibrutinib in many different patient-derived xenograft (PDX) samples, mostly representing high risk leukemia cases and covering a wide variety of ALL subtypes. In >80% of the cases, Applicants observed synergy ranging from moderate to strong with a combination index (CI)<0.8 (FIG. 5F, 5G).

Thus, it is an object of this disclosure to provide synergistic combinations of amino acid depletion agents (AADA, e.g. ASNase) and amino acid depletion agent sensitizers (AADAS, e.g. BTKi) for use in treating patients in need thereof.

It is a further object to provide methods of treating a subject or patient suffering from cancer comprising simultaneously or sequentially administering synergistically effective amounts of an AADA (e.g. ASNase) and an AADAS (e.g. BTKi). In some embodiments, the cancer may be a liquid or solid tumor. In some embodiments, the use of an AADAS may potentiate the solid tumor killing efficacy of otherwise ineffective amounts of AADA. In other embodiments, the AADAS may be combined with a better tolerated AADA, such as L-ASNase encapsulated in erythrocytes (e.g. Erytech's ERYASPASE®). Advantageous indications include, but are not limited to, solid tumor and lymphoma indications selected from Gastric cancer, Pancreatic cancer, NK Lymphoma, DLBCL, Colorectal cancer, Bladder cancer, Hepatic cancer and Glioma/Glioblastoma.

Determination of a synergistic interaction between an AADA and an AADAS may be based on the results obtained from the assays described herein. The results of these assays may be analyzed using the Chou and Talalay combination method and Dose-Effect Analysis with CalcuSyn software in order to obtain a Combination Index (Chou and Talalay, Trends Pharmacol. Sci. 4:450-454; Chou, T. C. (2006) Pharmacological Reviews 68(3):621-681; Chou and Talalay, 1984, Adv. Enzyme Regul. 22:27-55).

As further detailed in the Examples below, the synergistic AADA and AADAS combinations provided by this disclosure have been evaluated in several assay systems, and the data can be analyzed utilizing a standard program for quantifying synergism, additivism, and antagonism among anticancer agents. An exemplary program utilized is described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 to 1.2 indicate additive effects. The combination therapy may provide “synergy” and prove “synergistic”, i.e., the effect achieved when the active ingredients used together is greater than the sum of the effects that results from using the compounds separately. A “synergistic effect” may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In some embodiments, the synergistic combination therapy of this disclosure may be effective against tumor cells having one or more of the cytogenetic profiles indicated in FIG. 5F, 5G. For example, the synergistic combination therapy may be effective against tumor cells comprising the following: t(4;11) MLL-AF4, ETV6-RUNX1 (t(12;21); EBF1del1-16, PAX5del2 8, ETV6del1-2, IL3del, CSF2RA, lkzf1del4-7, E2A-HLF_t(17;19), E2a-PBX1; T(1;19), Ikzfh1del4-7, CDKN2Adel, ABL+, CDKN2A/BDel, PAX5Del, lkzf1 del1-8, P53Del, MLL, t(1;19), TP53 unknown (del-R248Q in relapse), TP53 del+R280T, E2a-PBX1; t(1;19), t(1;19) E2A-PBX1, t(2;12), complex.

In some embodiments, the synergistic combination therapy may be effective against solid tumor cells expressing or over-expressing a gene or mutated version thereof, whose deletion and/or inhibition sensitizes said cells to AADA-induced apoptosis. In some embodiments, the AADAS may be a BTK inhibitor and the AADA is ASNase.

In other embodiments, the synergistic combination therapy may be effective against a wide variety of solid tumor cells, including those from pancreatic cancer, breast cancer, colorectal cancer, gastric cancer, brain cancer, etc. The synergistic combination therapy may also be effective against semi-solid tumors including lymphomas. Advantageous indications include, but are not limited to, solid tumor and lymphoma indications selected from Gastric cancer, Pancreatic cancer, NK Lymphoma, DLBCL, Colorectal cancer, Bladder cancer, Hepatic cancer and Glioma/Glioblastoma.

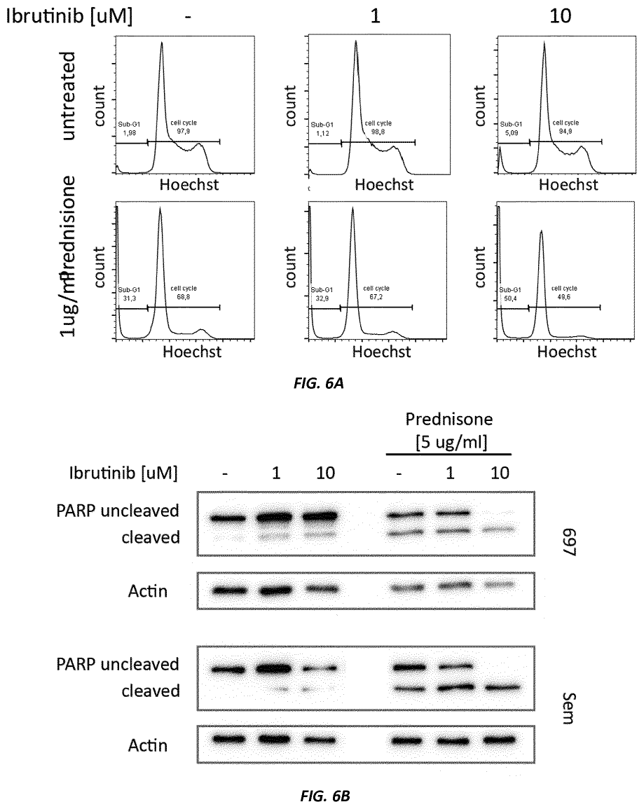

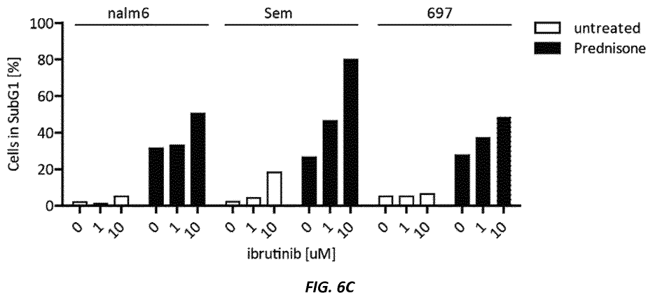

Furthermore, the data disclosed herein indicate that the synergistic effect of AADA (ASNase) and AADAS (BTKi) is selective for ASNase-induced cell death, since no clear synergy was seen between BTK inhibition and glucocorticoid-induced apoptosis (see Example 5 and FIGS. 6A to 6C). This finding was both surprising and unexpected, since skilled artisans might have reasonably expected the AADAS to sensitize cells to the action of other inducers of apoptosis, and not only AADA-induced apoptosis. Furthermore, as indicated above, inhibition of mTOR, another important component of the amino acid stress response route and known effector of BTK, did not affect response to AADA-induced apoptosis.

Now that the invention has been disclosed, the inventors envision that other combinations of AADAS and AADA will demonstrate comparable synergistic efficacy, and include Table 1 as a non-limiting list of such combinations. When the AADA is a peptidic agent, it may be present in any pharmaceutically acceptable form, including free, pegylated, otherwise conjugated and RBC encapsulated.

TABLE 1
Combinations of amino acid depletion agent sensitizer
(AADAS) and amino acid depletion agent (AADA)

| Combination | AADAS | AADA |
|---|---|---|
| 1 | BTKi | Asparaginase (ASNase) (e.g. GRASPA ®, |
| | | Erytech Pharma; ONCASPAR ®, Servier) |
| 2 | | ASN synthetase (ASNS) inhibitor |
| 3 | | Arginine deiminase (ADI) (e.g. ADI-PEG20, |
| | | POLARIS) |
| 4 | | Arginase (ARGase) |
| 5 | | Argininosuccinate synthetase inhibitor |
| | | (ASI) |
| 6 | | Adenosine deaminase (ADA) |
| 7 | | Methionase (METase) |
| 8 | | Glutaminase (GLNase) |
| 9 | | Glutamine synthetase inhibitor (GSI) |
| 10 | | ASNase + METase |
| 11 | | IDO (e.g. indoximod, docetaxel) |
| 12 | | TDO (e.g. indole LM10) |
| 13 | p21- | ASNase |
| 14 | activated | ASNS inhibitor |
| 15 | kinase | ADI |
| 16 | inhibitor | ARGase |
| 17 | (PAKi) | ASI |
| 18 | | ADA |
| 19 | | METase |
| 20 | | GLNase |
| 21 | | GSI |
| 22 | | ASNase + METase |
| 23 | | IDO |
| 24 | | TDO |
| 25 | Cell | ASNase |
| 26 | division | ASNS inhibitor |
| 27 | cycle 7 | ADI |
| 28 | inhibitor | ARGase |
| 29 | (CDC7i) | ASI |
| 30 | | ADA |
| 31 | | METase |
| 32 | | GLNase |
| 33 | | GSI |
| 34 | | ASNase + METase |
| 35 | | IDO |
| 36 | | TDO |
| 37 | TAOK2 | ASNase |
| 38 | inhibitor | ASNS inhibitor |
| 39 | | ADI |
| 40 | | ARGase |
| 41 | | ASI |
| 42 | | ADA |
| 43 | | METase |
| 44 | | GLNase |
| 45 | | GSI |
| 46 | | ASNase + METase |
| 47 | | IDO |
| 48 | | TDO |

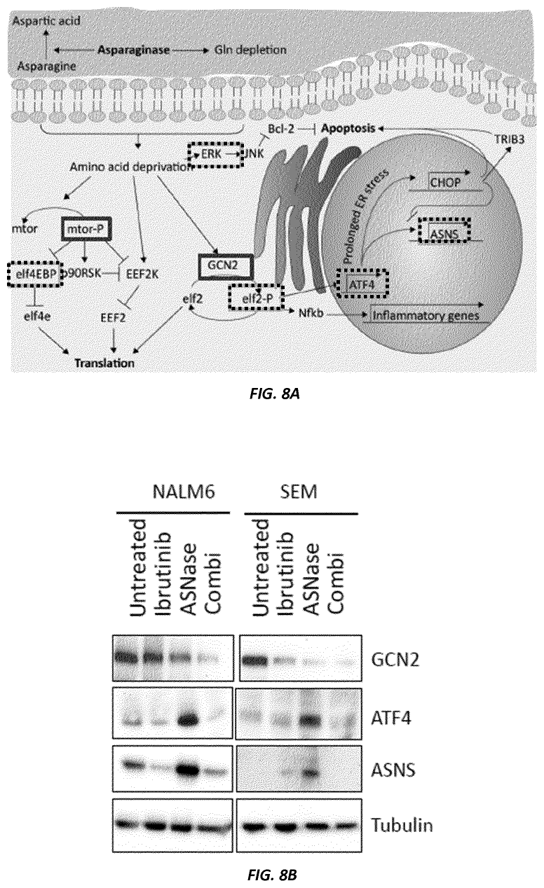

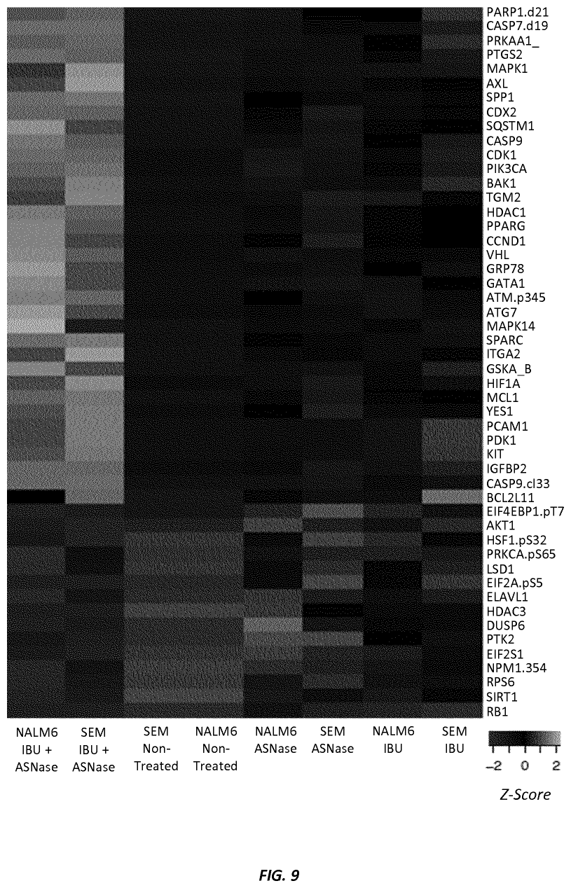

Moreover, the combination of ASNase and ibrutinib in a mouse xenograft model produced impressive clinical responses, even after a single block of treatment (9 days). See Example 6 below. One of the surprising observations was that the effects of ibrutinib (an inhibitor of a component of the B cell receptor signaling pathway, see FIG. 3A) were also seen in leukemia subtypes that do not (yet) express a rearranged (pre) B cell receptor. Mechanistically, these data indicate that inhibition of BTK leads to inhibition of the amino acid sensor GCN2 and the transcription factor ATF4 (see FIGS. 8A & 8B), a previously unrecognized connection between components of the B cell receptor signaling pathway and amino acid stress responses (FIG. 2A). As such, the invention also encompasses a method for overcoming ASNase resistance in patients or subjects comprising administering to patients synergistically effective amounts of a BTKi and an ASNase, such that the levels of the BTKi are sufficient to attenuate, block and/or prevent the ASNase-induced increases in ASNS expression, which would contribute to the ASNase resistance.

This unpredictable finding is further notable in that mice deficient for GCN2 show enhanced liver toxicity after exposure to ASNase, demonstrating that systemic inhibition of the amino acid stress response is not feasible (Phillipson-Weiner L et al. Am J Physiol Gastrointest Liver Physiol. 2016 Jun. 1; 310(11)) for combination with an AADA like ASNase. As such, the present disclosure demonstrates that it is possible to interfere with the GCN2-ATF4 stress response in a (tumor) cell type specific manner (i.e. because the biological target of AADAS is highly expressed in the target cells), reducing the chance of systemic toxicities.

By combining ASNase therapy with cell type specific inhibition of the GCN2-ATF4 signaling pathway, the therapeutic efficacy against ALL, other blood cancers, and solid tumors, may be enhanced while reducing side effects. In the case of ALL, this effect may be brought about by the inhibition of the hematopoietic cell specific kinase BTK, for instance by using the FDA-approved drug ibrutinib or other BTK inhibitors in combination with ASNase. Since ibrutinib is known to inhibit activation of the B cell receptor, an additional advantage could be the inhibition of antibody responses by normal B cells during treatment. Outside of ALL, the Applicants envision that sensitization of tumor cells to ASNase therapy may depend upon the expression level and/or activity of BTK (and/or related kinases of the TEC family) in such non-ALL tumor cells.

As used herein, the term “amino acid depletion agent sensitizer” (AADAS) means a drug, compound, biotherapeutic or other agent capable of sensitizing a tumor cell to treatment with an “amino acid depleting agent” (AADA).

As used herein, and “AADA” or “amino acid depleting agent” is any drug, compound, biotherapeutic or other agent capable of depleting amino acid levels to deprive one or more cell types of said amino acid. As disclosed herein, the AADAS and the AADA work together, in some embodiments synergistically, to induce apoptosis in one or more desired cell types.

In some embodiments, the amino acid depletion stress pathway is blocked using a Bruton's Tyrosine Kinase inhibitor (BTKi) (the AADAS) and an ASNase (the AADA).

In some embodiments, the ASNase is provided in the red blood cell (RBC)-encapsulated form currently produced Erytech Pharma. The RBCs are subjected to osmotic stress, which opens and reseals pores on the surface of the cells and allows the compounds to enter and be trapped inside the cells. Encapsulation offers a number of benefits as compared to free-form compounds.

The red blood cell membrane protects the therapeutic substance, which allows it to remain in the body longer and should lead to fewer administrations and a lower overall dose. The cellular membrane also protects the body against any direct toxicity of the drug substance, which can result in a decreased incidence of allergic reactions and other side effects. The combination of prolonged activity and reduced toxicity is particularly beneficial for the administration of certain therapeutic enzymes, that often have short half-lives and are associated with important toxicities. Examples include enzymes such as asparaginase (ASNase) and methioninase (METase), used to starve tumors by affecting cancer metabolism.

In some embodiments, the synergistic combination therapy may comprise a synergistically effective amount of an RBC-encapsulated ASNase and a synergistically effective amount of a BTKi.

In some embodiments, the synergistic combination therapy may comprise a synergistically effective amount of any ASNase, including but not limited to an Erwinase, a PEG-conjugated ASNase, or an ASNase lacking glutaminase activity, and a synergistically effective amount of a BTKi.

In some embodiments, treatment of a patient suffering from a cancer with either the synergistically effective amount of the ASNase or the synergistically effective amount of the BTKi alone results in inhibition of tumor cell proliferation, whereas treatment with the combination of ASNase and the BTKi induces massive tumor cell apoptosis.

In other embodiments, the BTKi blocks BTK from protecting tumor cells from ASNase-induced killing.

Furthermore, as is well-known, there are numerous ways to activate AA depletion-induced stress, including, but not limited to the following: 1) dietary restriction (see e.g. Erytech's International Patent Application WO2017/114966, disclosing novel methods of depleting MET and ASN for treating cancer); 2) ASN depletion (including ASNase, ASNS inhibitor, etc.); 3) MET depletion (e.g. METase, MGL, MET synthesis inhibitors); 4) TYR depletion (TDO, IDO inhibitors); 5) ARG depletion (ADI, ARGase . . . ); 6) Adenosine Deaminase (ADA); and/or 7) combinations thereof. Accordingly, the AADA may encompass any one or more of the foregoing AA depletion approaches, or any other means for activating the AA depletion stress response in cancer cells.

### Definitions

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

“Tumor,” as used herein, refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

The terms “cancer” and “cancerous” refer to or describe the physiological condition in mammals that is typically characterized by unregulated cell growth. Examples of cancer include, but are not limited to, blood cancers and solid tumors, including pancreatic ductal adenocarcinoma, colorectal cancer, breast cancer, triple negative breast cancer (TNBC), and B-cell lymphoproliferative disorders (BCLDs), such as lymphoma and leukemia. Particularly advantageous target indications include, but are not limited to, solid tumor and lymphoma indications selected from Gastric cancer, Pancreatic cancer, NK Lymphoma, DLBCL, Colorectal cancer, Bladder cancer, Hepatic cancer and Glioma/Glioblastoma.

By “refractory” in the context of a cancer is intended the particular cancer is resistant to, or non-responsive to, therapy with a particular therapeutic agent. A cancer can be refractory to therapy with a particular therapeutic agent either from the onset of treatment with the particular therapeutic agent (i.e., non-responsive to initial exposure to the therapeutic agent), or as a result of developing resistance to the therapeutic agent, either over the course of a first treatment period with the therapeutic agent or during a subsequent treatment period with the therapeutic agent.

By “agonist activity” is intended that a substance functions as an agonist. By “antagonist activity” is intended that the substance functions as an antagonist. An antagonist of “Bruton's Tyrosine Kinase” (BTK) prevents or reduces induction of any of the responses mediated by BTK.

In some embodiments, the BTK inhibitor therapeutic agent is an antagonist anti-BTK antibody. Such antibodies are free of significant agonist activity as noted above when bound to a BTK antigen in a human cell.

By “BTK-mediated signaling” it is intended any of the biological activities that are dependent on, either directly or indirection, the activity of BTK.

A BTK “signaling pathway” or “signal transduction pathway” is intended to mean at least one biochemical reaction, or a group of biochemical reactions, that results from the activity of BTK, and which generates a signal that, when transmitted through the signal pathway, leads to activation of one or more downstream molecules in the signaling cascade. Signal transduction pathways involve a number of signal transduction molecules that lead to transmission of a signal from the cell-surface across the plasma membrane of a cell, and through one or more in a series of signal transduction molecules, through the cytoplasm of the cell, and in some instances, into the cell's nucleus.

As used herein, the term “agonist” refers to a compound, the presence of which results in a biological activity of a protein that is the same as the biological activity resulting from the presence of a naturally occurring ligand for the protein, such as, for example, BTK.

As used herein, the term “partial agonist” refers to a compound the presence of which results in a biological activity of a protein that is of the same type as that resulting from the presence of a naturally occurring ligand for the protein, but of a lower magnitude.

As used herein, the term “antagonist” refers to a compound, the presence of which results in a decrease in the magnitude of a biological activity of a protein. In certain embodiments, the presence of an antagonist results in complete inhibition of a biological activity of a protein, such as, for example, BTK. In certain embodiments, an antagonist is an inhibitor.

The term “Bruton's tyrosine kinase (BTK),” as used herein, refers to Bruton's tyrosine kinase from Homo sapiens (e.g. GenBank Accession No. NP-000052). The term “Bruton's tyrosine kinase homolog,” as used herein, refers to orthologs of Bruton's tyrosine kinase, e.g., the orthologs from mouse (GenBank Accession No. AAB47246), dog (GenBank Accession No. XP-549139.), rat (GenBank Accession No. NP-001007799), chicken (GenBank Accession No. NP 989564), or zebra fish (GenBank Accession No. XP 698117), and fusion proteins of any of the foregoing that exhibit kinase activity towards one or more substrates of Bruton's tyrosine kinase (e.g. a peptide substrate having the amino acid sequence “AVLESEEELYSSARQ”).

The terms “co-administration” or “combination therapy” and the like, as used herein, are meant to encompass administration of the selected therapeutic agents to a single patient, and are intended to include treatment regimens in which the agents are administered by the same or different route of administration or at the same or different time.

The term “effective amount,” as used herein, refers to a sufficient amount of an AADA and an AADAS. In some embodiments, the AADAS may be a BTK inhibitory agent or a BTK inhibitor compound being administered which will result in an increase or appearance in the blood of a subpopulation of lymphocytes (e.g., pharmaceutical debulking). An appropriate “effective amount” in any case may be determined using techniques, such as a dose escalation study.

The term “therapeutically effective amount,” as used herein, refers to a sufficient amount of an agent or a compound being administered which will relieve to some extent one or more of the symptoms of a disease or condition. The result can be reduction and/or alleviation of the signs, symptoms, or causes of the disease or condition, or any other desired alteration of a biological system. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound of any of Formula (A), Formula (B), Formula (C), or Formula (D), age, weight, general condition of the subject, the condition being treated, the severity of the condition being treated, and the judgment of the prescribing physician. For examples, therapeutically effective amounts may be determined by routine experimentation, including but not solely dose escalation trials.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the physician.

The terms “inhibits”, “inhibiting”, or “inhibitor” of a kinase, as used herein, refer to inhibition of enzymatic phosphotransferase activity.

The term “irreversible inhibitor,” as used herein, refers to a compound that, upon contact with a target protein (e.g., a kinase) causes the formation of a new covalent bond with or within the protein, whereby one or more of the target protein's biological activities (e.g., phosphotransferase activity) is diminished or abolished notwithstanding the subsequent presence or absence of the irreversible inhibitor.

The term “irreversible BTK inhibitor,” as used herein, refers to an inhibitor of BTK that can form a covalent bond with an amino acid residue of BTK. In one embodiment, the irreversible inhibitor of BTK can form a covalent bond with a Cys residue of BTK; in particular embodiments, the irreversible inhibitor can form a covalent bond with a Cys 481 residue (or a homolog thereof) of BTK or a cysteine residue in the homologous corresponding position of another TK.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. For example, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes by which a substance is changed by an organism.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. In certain embodiments the presence of a modulator results in an activity that does not occur in the absence of the modulator.

As used herein, the term “selective binding compound” refers to a compound that selectively binds to any portion of one or more target proteins.

As used herein, the term “selectively binds” refers to the ability of a selective binding compound to bind to a target protein, such as, for example, BTK, with greater affinity than it binds to a non-target protein.

As used herein, the term “selective modulator” refers to a compound that selectively modulates a target activity relative to a non-target activity.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 75%, about 80%, about 85%, about 90%, about 95%, about 96%, about 97%, about 98%, about 99% or greater.

The term “subject” as used herein, refers to an animal (including a mammal, including a human), which is the object of treatment, observation or experiment.

As used herein, the term “target activity” refers to a biological activity capable of being modulated by a selective modulator. Certain exemplary target activities include, but are not limited to, binding affinity, signal transduction, enzymatic activity, tumor growth, effects on particular biomarkers related to B-cell lymphoproliferative disorder pathology.

As used herein, the term “target protein” refers to a molecule or a portion of a protein capable of being bound by a selective binding compound.

The terms “treat,” “treating” or “treatment”, as used herein, include alleviating, abating or ameliorating a disease or condition, or symptoms thereof; managing a disease or condition, or symptoms thereof; preventing additional symptoms; ameliorating or preventing the underlying metabolic causes of symptoms; inhibiting the disease or condition, e.g., arresting the development of the disease or condition; relieving the disease or condition; causing regression of the disease or condition, relieving a condition caused by the disease or condition; or stopping the symptoms of the disease or condition. The terms “treat,” “treating” or “treatment”, include, but are not limited to, prophylactic and/or therapeutic treatments.

As used herein, the IC₅₀ refers to an amount, concentration or dosage of a particular test compound or combination that achieves a 50% inhibition of a maximal response.

As used herein, EC₅₀ refers to a dosage, concentration or amount of a particular test compound or combination that elicits a dose-dependent response at 50% of maximal expression of a particular response that is induced, provoked or potentiated by the particular test compound or combination.

In some embodiments, the combinations of AADA and AADAS may be used in a method of treating a malignancy, including a hematological malignancy, in an subject in need thereof, comprising: (a) administering to the subject an amount of an AADAS (e.g. a BTKi) sufficient to sensitize a plurality of the malignant cells to treatment with an AADA (e.g. ASNase); and (b) administering to the subject an amount of an AADA (e.g. ASNase) sufficient to induce apoptosis in the malignant cells sensitized thereto by the AADAS. In some embodiments, the amount of the AADA and/or the AADAS would be subtherapeutic were either to be administered as a monotherapy (e.g. without the corresponding AADA or AADAS) for the treatment of said malignancy. As used herein, “Individually Subtherapeutic Amount” means that administration of the AADA or the AADAS absent its corresponding AADA or AADAS would be insufficient to treat a given disease or condition that could be effectively treated by a combination of the AADA and the AADAS. In some embodiments, one or both the AADA and the AADAS may be administered to a subject in individually subtherapeutic amounts. In some embodiments, both the AADA and the AADAS are administered to the subject in individually subtherapeutic amounts.

In some embodiments, the AADAS is a BTKi and the AADA is an ASNase. In such embodiments, the amount of the BTKi may be insufficient to induce death in all of the cells of the malignancy, but may be sufficient to sensitize at least a plurality of the cells to ASNase-induced apoptosis.

In some embodiments, the malignancy is a hematological malignancy including ALL, AML or CLL. In other embodiments, the malignancy is a solid tumor, including but not limited to CRC, TNBC, PANC or any other solid tumor. Advantageous target indications include solid tumor and lymphoma indications selected from Gastric cancer, Pancreatic cancer, NK Lymphoma, DLBCL, Colorectal cancer, Bladder cancer, Hepatic cancer and Glioma/Glioblastoma.

In some embodiments, the method comprises evaluating the extent to which the AADAS has sensitized the malignant cells to AADA-induced apoptosis. Evaluation may comprise measuring the level of inhibition or inactivation of a biological target of the AADAS. For example, when the biological target of the AADAS is BTK, the level of phosphorylation of BTK may be measured to determine whether a given cell has been sensitized to AADA-induced apoptosis. In some embodiments, administration of the AADA may be timed to maximize the synergistic efficacy of the combination of AADA and AADAS. For example, it may be clinically advantageous to ensure the maximum inhibition of a biological target (e.g. BTK) prior to administering the AADA (e.g. ASNase). In some embodiments, if the evaluation indicates that maximum sensitization has been achieved by the administration of the AADAS, the amount of the AADAS administered to the subject may be lowered.

As used herein, a “sensitization-effective amount” means an amount of AADAS sufficient to elicit sensitivity to AADA-induced apoptosis. Likewise, a “maximal sensitization-effective amount” means a sensitization-effective amount that is capable of eliciting the maximal sensitization effect for a given AADAS (e.g. a BTKi) to a given AADA (e.g. an ASNase).

In some embodiments, evaluating the extent of sensitization comprises measuring the duration of the reduction in activity of the biological target of the AADAS (e.g. phosphorylation status of BTK) as compared to before administration of the AADAS (e.g. a BTKi). In some embodiments, the method comprises administering the AADA after the activity of the biological target has remained at a predetermined reduced level for a predetermined length of time.

In some embodiments, the hematological malignancy is a chronic lymphocytic leukemia (CLL), small lymphocytic lymphoma (SLL), high risk CLL, or a non-CLL/SLL lymphoma. In some embodiments, the hematological malignancy is follicular lymphoma, diffuse large B-cell lymphoma (DLBCL), mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma. In some embodiments, the hematological malignancy is acute or chronic myelogenous (or myeloid) leukemia, myelodysplastic syndrome, or acute lymphoblastic leukemia. In some embodiments, the hematological malignancy is relapsed or refractory diffuse large B-cell lymphoma (DLBCL), relapsed or refractory mantle cell lymphoma, relapsed or refractory follicular lymphoma, relapsed or refractory CLL; relapsed or refractory SLL; relapsed or refractory multiple myeloma. In some embodiments, the hematological malignancy is a hematological malignancy that is classified as high-risk. In some embodiments, the hematological malignancy is high risk CLL or high risk SLL.

B-cell lymphoproliferative disorders (BCLDs) are neoplasms of the blood and encompass, inter alia, non-Hodgkin lymphoma, multiple myeloma, and leukemia. BCLDs can originate either in the lymphatic tissues (as in the case of lymphoma) or in the bone marrow (as in the case of leukemia and myeloma), and they all are involved with the uncontrolled growth of lymphocytes or white blood cells. There are many subtypes of BCLD, e.g., chronic lymphocytic leukemia (CLL) and non-Hodgkin lymphoma (NHL). The disease course and treatment of BCLD is dependent on the BCLD subtype; however, even within each subtype the clinical presentation, morphologic appearance, and response to therapy is heterogeneous.

Malignant lymphomas are neoplastic transformations of cells that reside predominantly within lymphoid tissues. Two groups of malignant lymphomas are Hodgkin's lymphoma and non-Hodgkin's lymphoma (NHL).

In some embodiments, the combinations of AADA and AADAS may be used in a method of treating any malignancy, including a solid tumor, a hematological malignancy, a BCLD or a malignant lymphoma.

Disclosed herein, in certain embodiments, is a method for treating a DLCBL in a subject in need thereof, comprising administering a sensitization-effective amount of an AADAS (e.g. a BTKi) and an amount of AADA (e.g. an ASNase) sufficient to induce cell death (e.g. apoptosis) in DLCBL cells sensitized by said AADAS.

Disclosed herein, in certain embodiments, is a method for treating diffuse large B-cell lymphoma, activated B cell-like subtype (ABC-DLBCL), in a subject in need thereof, comprising administering a sensitization-effective amount of an AADAS (e.g. a BTKi) and an amount of AADA (e.g. an ASNase) sufficient to induce cell death (e.g. apoptosis) in ABC-DLBCL cells sensitized by said AADAS.

Disclosed herein, in certain embodiments, is a method for treating follicular lymphoma, in a subject in need thereof, comprising administering a sensitization-effective amount of an AADAS (e.g. a BTKi) and an amount of AADA (e.g. an ASNase) sufficient to induce cell death (e.g. apoptosis) in follicular lymphoma cells sensitized by said AADAS. As used herein, the term “follicular lymphoma” refers to any of several types of NHL in which the lymphomatous cells are clustered into nodules or follicles.

Disclosed herein, in certain embodiments, is a method for treating a CLL or SLL in an individual in need thereof, comprising administering a sensitization-effective amount of an AADAS (e.g. a BTKi) and an amount of AADA (e.g. an ASNase) sufficient to induce cell death (e.g. apoptosis) in CLL or SLL cells sensitized by said AADAS.

Disclosed herein, in certain embodiments, is a method for treating a Mantle cell lymphoma in an individual in need thereof, comprising administering a sensitization-effective amount of an AADAS (e.g. a BTKi) and an amount of AADA (e.g. an ASNase) sufficient to induce cell death (e.g. apoptosis) in Mantle cell lymphoma cells sensitized by said AADAS.

As used herein, the term, “Mantle cell lymphoma” (MCL) refers to a subtype of B-cell lymphoma, due to CD5 positive antigen-naive pregerminal center B-cell within the mantle zone that surrounds normal germinal center follicles.

Disclosed herein, in certain embodiments, is a method for treating a marginal zone B-cell lymphoma in an individual in need thereof, comprising administering a sensitization-effective amount of an AADAS (e.g. a BTKi) and an amount of AADA (e.g. an ASNase) sufficient to induce cell death (e.g. apoptosis) in marginal zone B-cell lymphoma cells sensitized by said AADAS.

As used herein, the term “marginal zone B-cell lymphoma” refers to a group of related B-cell neoplasms that involve the lymphoid tissues in the marginal zone, the patchy area outside the follicular mantle zone.

Disclosed herein, in certain embodiments, is a method for treating a “mucosa-associated lymphoid tissue” (MALT) lymphoma in an individual in need thereof, comprising administering a sensitization-effective amount of an AADAS (e.g. a BTKi) and an amount of AADA (e.g. an ASNase) sufficient to induce cell death (e.g. apoptosis) in MALT cells sensitized by said AADAS.

Disclosed herein, in certain embodiments, is a method for treating a nodal marginal zone B-cell lymphoma in an individual in need thereof, comprising administering a sensitization-effective amount of an AADAS (e.g. a BTKi) and an amount of AADA (e.g. an ASNase) sufficient to induce cell death (e.g. apoptosis) in marginal zone B cell lymphoma cells sensitized by said AADAS.

Disclosed herein, in certain embodiments, is a method for treating a Splenic Marginal Zone B-Cell Lymphoma in an individual in need thereof, comprising administering a sensitization-effective amount of an AADAS (e.g. a BTKi) and an amount of AADA (e.g. an ASNase) sufficient to induce cell death (e.g. apoptosis) in Splenic Marginal Zone B-Cell Lymphoma cells sensitized by said AADAS.

Disclosed herein, in certain embodiments, is a method for treating a Burkitt Lymphoma in an individual in need thereof, comprising administering a sensitization-effective amount of an AADAS (e.g. a BTKi) and an amount of AADA (e.g. an ASNase) sufficient to induce cell death (e.g. apoptosis) in Burkitt Lymphoma cells sensitized by said AADAS.

The term “Burkitt lymphoma” refers to a type of Non-Hodgkin Lymphoma (NHL) that commonly affects children. It is a highly aggressive type of B-cell lymphoma that often starts and involves body parts other than lymph nodes. In spite of its fast-growing nature, Burkitt's lymphoma is often curable with modern intensive therapies.

Disclosed herein, in certain embodiments, is a method for treating a Waldenstrom Macroglobulinemia in an individual in need thereof, comprising administering a sensitization-effective amount of an AADAS (e.g. a BTKi) and an amount of AADA (e.g. an ASNase) sufficient to induce cell death (e.g. apoptosis) in Waldenstrom Macroglobulinemia cells sensitized by said AADAS.

The term “Waldenstrom macroglobulinemia”, also known as lymphoplasmacytic lymphoma, is cancer involving a subtype of white blood cells called lymphocytes. It is characterized by an uncontrolled clonal proliferation of terminally differentiated B lymphocytes, and by the lymphoma cells making an antibody called immunoglobulin M (IgM).

Disclosed herein, in certain embodiments, is a method for treating a Multiple Myeloma in an individual in need thereof, comprising administering a sensitization-effective amount of an AADAS (e.g. a BTKi) and an amount of AADA (e.g. an ASNase) sufficient to induce cell death (e.g. apoptosis) in Multiple Myeloma cells sensitized by said AADAS.

Disclosed herein, in certain embodiments, is a method for treating a solid tumor or lymphoma in an individual in need thereof, comprising administering a sensitization-effective amount of an AADAS (e.g. a BTKi) and an amount of AADA (e.g. an ASNase) sufficient to induce cell death (e.g. apoptosis) in solid tumor or lymphoma cells sensitized by said AADAS. The methods may be effective against any solid tumor or lymphoma, including those selected from Gastric cancer, Pancreatic cancer, NK Lymphoma, Colorectal cancer, Bladder cancer, Hepatic cancer and Glioma/Glioblastoma.

Disclosed herein, in certain embodiments, is a method for treating a Leukemia in an individual in need thereof, comprising administering a sensitization-effective amount of an AADAS (e.g. a BTKi) and an amount of AADA (e.g. an ASNase) sufficient to induce cell death (e.g. apoptosis) in Leukemia cells sensitized by said AADAS.

Leukemia is a cancer of the blood or bone marrow characterized by an abnormal increase of blood cells, usually leukocytes (white blood cells). Leukemia is a broad term covering a spectrum of diseases. The first division is between its acute and chronic forms: (i) acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children; (ii) chronic leukemia is distinguished by the excessive build-up of relatively mature, but still abnormal, white blood cells. Leukemia includes, but not limited to, Acute lymphoblastic leukemia (ALL), Acute myelogenous leukemia (AML), Chronic myelogenous leukemia (CML), and Hairy cell leukemia (HCL).

Disclosed herein, in certain embodiments, is a method for treating a primary central nervous system lymphoma in an individual in need thereof, comprising administering a sensitization-effective amount of an AADAS (e.g. a BTKi) and an amount of AADA (e.g. an ASNase) sufficient to induce cell death (e.g. apoptosis) in primary central nervous system lymphoma cells sensitized by said AADAS.

Disclosed herein, in certain embodiments, is a method for treating an extranodal natural killer (NK) cell/T-cell lymphoma (ENKTL) in an individual in need thereof, comprising administering a sensitization-effective amount of an AADAS (e.g. a BTKi) and an amount of AADA (e.g. an ASNase) sufficient to induce cell death (e.g. apoptosis) in extranodal natural killer (NK) cell/T-cell lymphoma (ENKTL) cells sensitized by said AADAS.

In some embodiments, the AADAS is a BTK inhibitor selected from the group consisting of a small organic molecule, a macromolecule, a peptide or a non-peptide.

In some embodiments, the BTK inhibitor provided herein is a reversible or irreversible inhibitor. In certain embodiments, the BTK inhibitor is an irreversible inhibitor.

In some embodiments, the irreversible BTK inhibitor forms a covalent bond with a cysteine sidechain of a Bruton's tyrosine kinase, a Bruton's tyrosine kinase homolog, or a BTK tyrosine kinase cysteine homolog.

Irreversible BTK inhibitor compounds can use for the manufacture of a medicament for treating any of the foregoing conditions (e.g., autoimmune diseases, inflammatory diseases, allergy disorders, B-cell proliferative disorders, or thromboembolic disorders).

In some embodiments, the BTK inhibitor compound used for the methods described herein inhibits BTK or a BTK homolog kinase activity with an in vitro IC50 of less than about 10 μM. (e.g., less than about 1 μM, less than about 0.5 μM, less than about 0.4 μM, less than about 0.3 μM, less than about 0.1, less than about 0.08 μM, less than about 0.06 μM, less than about 0.05 μM, less than about 0.04 μM, less than about 0.03 μM, less than about 0.02 μM, less than about 0.01, less than about 0.008 μM, less than about 0.006 μM, less than about 0.005 μM, less than about 0.004 μM, less than about 0.003 μM, less than about 0.002 μM, less than about 0.001, less than about 0.00099 μM, less than about 0.00098 μM, less than about 0.00097 μM, less than about 0.00096 μM, less than about 0.00095 μM, less than about 0.00094 μM, less than about 0.00093 μM, less than about 0.00092, or less than about 0.00090 μM).

In one embodiment, the BTK inhibitor compound selectively and irreversibly inhibits an activated form of its target tyrosine kinase (e.g., a phosphorylated form of the tyrosine kinase). For example, activated BTK is transphosphorylated at tyrosine 551. Thus, in these embodiments the irreversible BTK inhibitor inhibits the target kinase in cells only once the target kinase is activated by the signaling events.

In some embodiments, the BTKi is selected from the following: BTKi is selected from ibrutinib (U.S. Pat. No. 7,514,444 to Pharmacyclics), acalabrutinib (U.S. Pat. No. 9,522,917 to Acerta), zanabrutinib (U.S. Pat. No. 9,447,106 to BeiGene), tirabrutinib (U.S. Pat. No. 8,557,803 to Ono), M7583 (U.S. Pat. No. 9,073,947 to Merck), vecabrutinib (U.S. Pat. No. 8,785,440 to Sunesis), CT-1530 (U.S. Pat. No. 9,447,106 to Centaurus), ARQ 531 (U.S. Pat. No. 9,630,968 to ArQule), DTRMWXHS-12 (U.S. Pat. No. 9,717,745 to Zhejiang), TG-1701 (WO 2017/118277 to TG Therapeutics), spebrutinib, CC-292 (U.S. Pat. No. 7,989,465 to Celgene), LOXO-305 (US 2017/0129897 to Loxo Oncology), CG′806 (Aptose Biosciences), evorbrutinib (U.S. Pat. No. 9,073,947 to Merck), RG7845, GDC-0853 (U.S. Pat. No. 8,716,274 to Roche-Genentech), poseltinib, LY3337641 and HM71224 (U.S. Pat. No. 8,957,065 to Lilly-Hanmi), PRN1008 (U.S. Pat. No. 9,580,427 to Principia), BMS-986142 (U.S. Pat. No. 8,846,673 to BMS), PRN2246 (U.S. Pat. No. 9,580,427 to Principia), TAK-020 (U.S. Pat. No. 9,365,566 to Takeda), AC0058 (U.S. Pat. No. 9,464,089 to Acea Biosciences), BIIB-068, vecabrutinib (U.S. Pat. No. 8,785,440 to Biogen) and combinations or equivalents thereof.

### Compositions, Formulations, and Routes of Administration

Also disclosed is a pharmaceutical composition comprising a disclosed AADA/AADAS combination in a pharmaceutically acceptable carrier. The composition may be a kit comprising effective amounts of the AADA and AADAS, and optionally instructions for use thereof.

Pharmaceutical compositions containing the disclosed AADA/AADAS combination can be administered to a patient using standard techniques. Techniques and formulations generally may be found in Remington's Pharmaceutical Sciences, 18th ed., Mack Publishing Co., Easton, Pa., 1990 (herein incorporated by reference).

Suitable dosage forms, in part, depend upon the use or the route of entry, for example, oral, transdermal, transmucosal, or by injection (parenteral, IV, etc.). Such dosage forms should allow the therapeutic agent to reach a target cell or otherwise have the desired therapeutic effect. For example, pharmaceutical compositions injected into the blood stream preferably are soluble. The disclosed conjugates and/or pharmaceutical compositions can be formulated as pharmaceutically acceptable salts and complexes thereof.

Pharmaceutically acceptable salts are non-toxic salts present in the amounts and concentrations at which they are administered. The preparation of such salts can facilitate pharmaceutical use by altering the physical characteristics of the compound without preventing it from exerting its physiological effect. Useful alterations in physical properties include lowering the melting point to facilitate transmucosal administration and increasing solubility to facilitate administering higher concentrations of the drug. The pharmaceutically acceptable salt of an AADA (e.g. an ASNase) may be present as a complex, as those in the art will appreciate.

Pharmaceutically acceptable salts include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate, and quinate. Pharmaceutically acceptable salts can be obtained from acids, including hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, supra. Such salts can be prepared using the appropriate corresponding bases.

Pharmaceutically acceptable carriers and/or excipients can also be incorporated into a pharmaceutical composition according to the invention to facilitate administration of the particular AADA or AADAS. Examples of carriers suitable for use in the practice of the invention include calcium carbonate, calcium phosphate, various sugars such as lactose, glucose, or sucrose, or types of starch, cellulose derivatives, gelatin, vegetable oils, polyethylene glycols, and physiologically compatible solvents. Examples of physiologically compatible solvents include sterile solutions of water for injection (WFI), saline solution and dextrose.

Pharmaceutical compositions can be administered by different routes, including intravenous, intraperitoneal, subcutaneous, intramuscular, oral, topical (transdermal), or transmucosal administration. For oral administration, for example, the compounds can be formulated into conventional oral dosage forms such as capsules, tablets, and liquid preparations such as syrups, elixirs, and concentrated drops. For injection, pharmaceutical compositions are formulated in liquid solutions, preferably in physiologically compatible buffers or solutions, such as saline solution, Hank's solution, or Ringer's solution.

In addition, the compounds may be formulated in solid form and re-dissolved or suspended immediately prior to use. For example, lyophilized forms of the conjugate can be produced. In a specific embodiment, the conjugate is administered intramuscularly. In another specific embodiment, the conjugate is administered intravenously. In some embodiments the pharmaceutical composition is contained in a vial as a lyophilized powder to be reconstituted with a solvent.

Systemic administration can also be accomplished by transmucosal or transdermal means. For transmucosal or transdermal administration, penetrants appropriate to the barrier to be permeated are used in the formulation. Such penetrants are well known in the art, and include, for example, for transmucosal administration, bile salts, and fusidic acid derivatives. In addition, detergents may be used to facilitate permeation. Transmucosal administration, for example, may be through nasal sprays, inhalers (for pulmonary delivery), rectal suppositories, or vaginal suppositories. For topical administration, compounds can be formulated into ointments, salves, gels, or creams, as is well known in the art.

The amounts of the composition to be delivered will depend on many factors, for example, the IC₅₀, EC₅₀, the biological half-life of the compound, the age, size, weight, and physical condition of the subject or patient, and the disease or disorder to be treated. The importance of these and other factors to be considered are well known to those of ordinary skill in the art. Generally, the amount of the composition (e.g. when the AADA is an enzyme like asparaginase) to be administered will range from about 10 International Units per square meter of the surface area of the patient's body (IU/m²) to 50,000 IU/m², with a dosage range of about 1,000 IU/m² to about 15,000 IU/m² being preferred, and a range of about 6,000 IU/m² to about 15,000 IU/m² being more preferred, and a range of about 10,000 to about 15,000 IU/m² (about 20-30 mg protein/m) being particularly preferred to treat a malignant hematologic disease, e.g., leukemia. Such dosages may be administered via intramuscular or intravenous injection at an interval of about 3 times weekly to about once per month, or once per week or once every other week during the course of therapy. Of course, other dosages and/or treatment regimens may be employed, as determined by the attending physician.

Nevertheless, it will be understood that various modifications may be made without departing from the spirit and scope of the invention. Accordingly, other embodiments are within the scope of the following claims.

### EXAMPLES

### Example 1—CRISPR/Cas9 Based Kinome Screen Identifies BTK as an Important Determinant of Asparaginase Treatment Response in Acute Lymphoblastic Leukemia (ALL)

Unless otherwise specified below, the in vitro studies were conducted using native E. coli ASNase (NCBI WP_000394140.1), and in vivo studies were conducted using ONCASPAR®. Moreover, all references to commercial product specifications should be interpreted to mean as understood by the skilled artisan as of the time of the filing of this application.

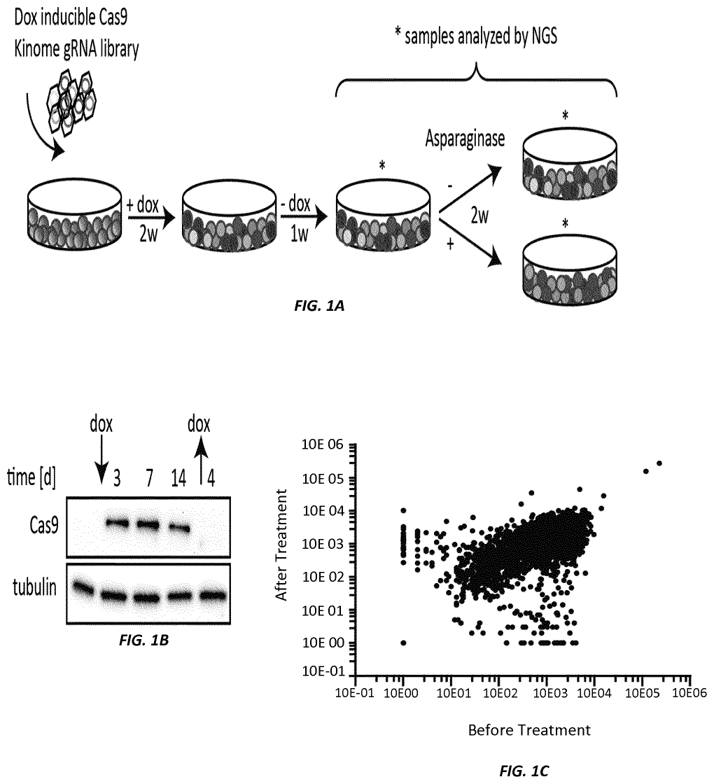

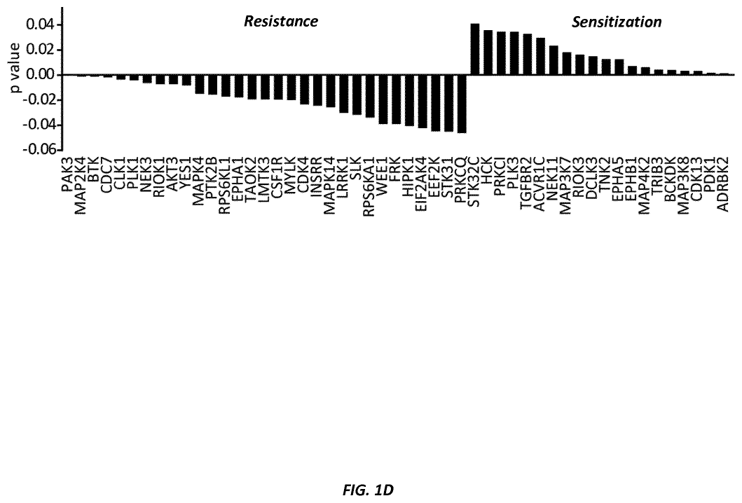

Briefly, a CRISPR/Cas9-based kinome screen (outlined in FIG. 1A) was conducted to identify genes whose deletion would have an impact on the ALL cells' sensitivity to amino acid depletion by ASNase. Nalm6 pre-B ALL cells were transduced with a lentivirus encoding a doxycycline inducible Cas9. Subsequently, a gRNA library targeting 506 kinases was introduced by lentiviral transduction with an average of one gRNA per cell. Each individual kinase in the library was represented by 10 gRNAs. After selection of transduced cells, doxycycline was added for one week to allow Cas9 expression to induce mutations. Thereafter, expression of Cas9 in response to doxycycline exposure was verified by Western Blotting (FIG. 1B). After verification of library complexity, cells were split for ASNase or mock treatment. Following treatment, DNA was harvested and subjected to NGS based sequencing to determine relative frequencies of each individual gRNA in the ASNase and control treated cells. and the distribution of gRNAs in the pool of Nalm6 cells before and after treatment with Asparaginase is shown in FIG. 1C. A deviation from the diagonal indicates enrichment (left) or depletion (right) of a specific gRNA during treatment. Finally, FIG. 1D indicates genes significantly enriched/depleted during asparaginase treatment.

Conclusion: These results show that the CRISPR-Cas9 based reverse genetics screen successfully identifies gene products that modulate the sensitivity to asparaginase treatment.

### Example 2—ASNase Response is Affected by Kinases Regulating the ER Stress Response Pathway

A series of experiments was conducted to begin to understand the mechanisms involved in the observed gene-deletion-mediated sensitization or resistance to amino acid starvation stress induced by ASNase.

FIG. 2A presents a schematic overview of the amino acid response pathway. Kinases representing gRNAs enriched in the screen are shown in blue, and kinases representing gRNAs that are selectively lost (dropouts) are shown in purple. Targeted knockout experiments were used to validate these results in independent experiments. As indicated by the Western Blot data shown in FIG. 2B, the TRIB3 gene was effectively disrupted by CRISPR/Cas9 mediated introduction of frame shift mutations/deletions in a pool of Nalm6 cells. In another pool of Nalm6 cells, the GCN2 gene was similarly disrupted by CRISPR/Cas9 mediated introduction of frame shift mutations/deletions (FIG. 2C). Nalm6 wt and TRB3 knockout cells (pool) were then left untreated or treated with ASNase and their proliferation was determined by measuring cell numbers over time (FIGS. 2D & 2E), the SubG1 (apoptotic) fraction was determined by Hoechst staining (FIG. 2F). Additionally, viability was measured using a MTT based viability assay (FIG. 2G). Nalm6 control and GCN2 deleted cells (pools) were either left untreated or treated with ASNase and the SubG1 (apoptotic) fraction was determined by Hoechst staining (FIG. 2H). Asterisks indicates a significant difference: * p<0.05, ** p<0.01, *** p<0.001. Viability was measured using a MTT based viability assay (FIG. 2I).

Conclusions: These results indicate that two candidate genes that were identified with the reverse genetic screen, TRB3 and GCN2, previously implicated in the amino acid stress response pathway, are indeed valid modifiers of the response to asparaginase.

### Example 3—Targeted Deletion or Pharmacological Inhibition of BTK Sensitizes Nalm6 Cells to ASNase Treatment

A series of experiments was conducted to study the impact of deletion or pharmacological inhibition of Bruton's Tyrosine Kinase (BTK) on the response of cells to ASNase treatment.

BTK Deletion. FIG. 3A presents a schematic overview depicting the (pre-) B cell receptor pathway. Initially, the BTK gene was disrupted by CRISPR/Cas9 mediated introduction of frame shift mutations/deletions in Nalm6 cells and single cell clones were evaluated for BTK expression using Western blot (FIG. 3C). Nalm6 wt Clones and BTK knockout clones were then either treated with 1 or 5 IU/ml ASNase or left untreated for 1 week before the SubG1 (apoptotic) fraction was measured by Flow cytometry. (FIGS. 3D & 3E) The same cells were also analyzed for the presence of apoptotic cells by Hoechst staining (FIGS. 3G & 3H). Asterisks indicate a significant difference: * p<0.05, ** p<0.01, *** p<0.001.

BTK inhibition. Wildtype Nalm6, Sem and Reh cells were cultured for 7 days in the presence or absence of 1 IU/ml ASNase and increasing concentrations of the BTK inhibitor ibrutinib. After 7 days, drugs were washed out and remaining cells reseeded in soft agarose containing medium (scheme presented in FIG. 4A). Cell viability was then measured by cell cycle distribution, (FIGS. 4C & 4D), and by blotting for the apoptosis marker PARP and calculating the ratio between cleaved and uncleaved PARP protein or (FIG. 4E) by amine staining (FIG. 4F). After a washout of ibrutinib and ASNase, viable cells were maintained in semi soft agar for three days and colonies were stained with Cristal violet and quantified by cell counting (FIGS. 4G & 4H). Finally, FIG. 4I is a model explaining how the asparaginase/ibrutinib combination therapy shifts cells from a survival mode into an apoptosis mode.

Conclusions. The ability of the combination of ibrutinib and ASNase to elicit such a significant apoptotic killing efficacy was surprising and unexpected, particularly in view of the relatively small effect that ibrutinib and ASNase exerted against cells when used alone (FIGS. 4D, 4E, 4F & 4H).

### Example 4—ASNase and Ibrutinib Exhibit Synergistic Efficacy in ALL Cell Lines and ALL Xenografts

A series of experiments was conducted to test the extent of synergistic efficacy between ASNase and ibrutinib against patient-derived xenografts (PDX). The viability and proliferative capacity of primary human ALL cells outside of the body is extremely poor. In order to expand the number of tumor cells, human ALL cells are injected into the bone marrow of immunocompromised mice. After 2-4 months these mice developed full blown leukemia with an immunophenotype and genetic composition that in the large majority of cases is identical to the original material at the time of injection. These cells can then be isolated and used immediately or viably frozen for future use in in vitro and in vivo experiments. These PDX cells were used to test for synergistic effects between Ibrutinib and asparaginase of cell viability (FIGS. 5A-5G).

FIG. 5A presents a schematic overview representing the workflow used to derive drug synergies in ALL cell lines and PDX samples. Briefly, hTERT immortalized MSCs were seeded in either a 384 wells format (microscopy) or a 96 wells format (7AAD staining) and allowed to settle for 24 hours prior to the addition of ALL xenografts. Cells were again allowed to settle for 24 hours before ASNase and ibrutinib were added as serial dilutions. After 3 and 7 days of incubation, cell death was analyzed using microscopy using live cells staining or flowcytometry using 7AAD to discriminate between live or dead cells. Quantification of the microscopy images was done using matlab. In a second step the data were analyzed using R-software to calculate a Combination Index (CI). For 7 day studies, cells were refed midway with ASN-depleted media.

Using this workflow, 73 experiments were performed, using 35 unique PDX samples. A total of 59 experiments passed quality control and were submitted for analysis. A strong synergy between asparaginase and Ibrutinib was found in over 70% of experiments (CI 0.8) (FIG. 5C). FIG. 5D shows representative examples of synergistic, additive and antagonistic effects of the combination treatment obtained using the automated microscopy. Of note, antagonistic effects were only observed in those cases that already showed a strong response to asparaginase alone. These results were validated in an independent experiment using flowcytometry based evaluation of cell viability after treatment. FIG. 5E shows the response of 10 different PDX samples to combination treatment. FIG. 5F is a summary table indicating the synergistic killing efficacy of the combination against PDX; and FIG. 5G is a summary table showing the synergistic killing efficacy, but this time, with the 100 μM ibrutinib group excluded from the CI calculation.

Conclusion: A strong synergy between asparaginase and Ibrutinib in (ex vivo) tumor cell killing was observed in over 70% of ALL cases (PDX).

For the following Tables: A=ASNase; A0=0; A1=0.01; A2=0.03; A3=0.1; A4=0.3; A5=1; A6=3; A7=10; A8=32; A=100; B=ibrutinib; B0=0; B1=0.001; B2=0.004; B3=0.02; B4=0.1; B5=0.3; B6=1; B7=6; B8=24; B9=100.

TABLE 2
m3810 (7 d)

| | A0 | A1 | A2 | A3 | A4 | A5 | A6 | A7 | A8 | A9 |
|---|---|---|---|---|---|---|---|---|---|---|
| B9 | 49 | 49 | 50 | 32 | 32 | 38 | 67 | 36 | 7 | 3 |
| B8 | 67 | 69 | 53 | 64 | 55 | 50 | 58 | 70 | 32 | 2 |
| B7 | 78 | 87 | 67 | 80 | 50 | 66 | 91 | 49 | 41 | 1 |
| B6 | 85 | 105 | 77 | 121 | 87 | 102 | 108 | 122 | 42 | 2 |
| B5 | 163 | 151 | 153 | 116 | 115 | 144 | 91 | 81 | 27 | 2 |
| B4 | 373 | 286 | 334 | 301 | 220 | 243 | 198 | 90 | 44 | 3 |
| B3 | 529 | 383 | 494 | 411 | 369 | 329 | 274 | 126 | 55 | 4 |
| B2 | 564 | 429 | 500 | 442 | 376 | 304 | 261 | 138 | 49 | 3 |
| B1 | 563 | 492 | 505 | 418 | 476 | 379 | 302 | 100 | 63 | 4 |
| B0 | 2527 | 1776 | 1752 | 1563 | 1519 | 1136 | 1251 | 686 | 87 | 4 |

TABLE 3
m764 (3 d)

| | A0 | A1 | A2 | A3 | A4 | A5 | A6 | A7 | A8 | A9 |
|---|---|---|---|---|---|---|---|---|---|---|
| B9 | 24 | 16 | 21 | 32 | 41 | 32 | 17 | 18 | 18 | 7 |
| B8 | 52 | 81 | 53 | 82 | 66 | 98 | 55 | 63 | 57 | 4 |
| B7 | 76 | 92 | 82 | 109 | 105 | 124 | 67 | 110 | 79 | 4 |
| B6 | 74 | 124 | 88 | 87 | 103 | 78 | 93 | 59 | 46 | 8 |
| B5 | 111 | 107 | 167 | 113 | 104 | 128 | 117 | 82 | 146 | 5 |
| B4 | 123 | 113 | 114 | 119 | 102 | 101 | 152 | 73 | 85 | 5 |
| B3 | 111 | 111 | 103 | 142 | 118 | 129 | 136 | 99 | 75 | 10 |
| B2 | 105 | 114 | 124 | 113 | 120 | 92 | 138 | 92 | 145 | 3 |
| B1 | 112 | 92 | 116 | 104 | 85 | 114 | 139 | 94 | 73 | 7 |
| B0 | 697 | 546 | 1126 | 109 | 250 | 915 | 887 | 594 | 157 | 2 |

TABLE 4
m838 (3 d)

| | A0 | A1 | A2 | A3 | A4 | A5 | A6 | A7 | A8 | A9 |
|---|---|---|---|---|---|---|---|---|---|---|
| B9 | 249 | 430 | 397 | 508 | 641 | 254 | 245 | 50 | 40 | 16 |
| B8 | 628 | 1082 | 924 | 986 | 1084 | 997 | 476 | 372 | 18 | 14 |
| B7 | 1014 | 992 | 1024 | 1214 | 1215 | 1227 | 798 | 532 | 47 | 14 |
| B6 | 1169 | 749 | 1233 | 1225 | 1192 | 716 | 1101 | 747 | 61 | 13 |
| B5 | 1019 | 1074 | 1148 | 1361 | 1334 | 1369 | 1143 | 527 | 67 | 10 |
| B4 | 1638 | 938 | 1479 | 1605 | 1643 | 925 | 1264 | 706 | 72 | 11 |
| B3 | 1765 | 1476 | 1447 | 1589 | 1294 | 1385 | 1241 | 949 | 83 | 12 |
| B2 | 1646 | 1465 | 1759 | 1002 | 993 | 1424 | 918 | 876 | 99 | 7 |
| B1 | 1577 | 911 | 1662 | 1755 | 1125 | 789 | 1437 | 678 | 173 | 15 |
| B0 | 3112 | 2793 | 3003 | 3109 | 2839 | 2454 | 3049 | 2431 | 175 | 18 |

TABLE 5
m1095 (3 d)

| | A0 | A1 | A2 | A3 | A4 | A5 | A6 | A7 | A8 | A9 |
|---|---|---|---|---|---|---|---|---|---|---|
| B9 | 420 | 408 | 342 | 333 | 335 | 289 | 251 | 224 | 40 | 12 |
| B8 | 813 | 860 | 826 | 708 | 763 | 777 | 806 | 518 | 56 | 19 |
| B7 | 773 | 887 | 747 | 703 | 611 | 770 | 689 | 620 | 174 | 13 |
| B6 | 831 | 919 | 902 | 683 | 779 | 906 | 751 | 584 | 153 | 23 |
| B5 | 858 | 695 | 739 | 686 | 878 | 726 | 865 | 606 | 136 | 17 |
| B4 | 853 | 834 | 914 | 812 | 716 | 660 | 805 | 494 | 198 | 9 |
| B3 | 745 | 787 | 754 | 734 | 814 | 765 | 701 | 534 | 144 | 13 |
| B2 | 758 | 753 | 723 | 785 | 721 | 616 | 481 | 352 | 174 | 18 |
| B1 | 747 | 749 | 724 | 658 | 551 | 603 | 557 | 320 | 312 | 16 |
| B0 | 3148 | 2935 | 2914 | 3089 | 3205 | 2579 | 549 | 2033 | 238 | 30 |

TABLE 6
m4136 (3 d)

| | A0 | A1 | A2 | A3 | A4 | A5 | A6 | A7 | A8 | A9 |
|---|---|---|---|---|---|---|---|---|---|---|
| B9 | 100 | 122 | 85 | 165 | 122 | 128 | 92 | 64 | 27 | 13 |
| B8 | 132 | 94 | 95 | 141 | 105 | 130 | 114 | 177 | 79 | 17 |
| B7 | 158 | 126 | 95 | 164 | 113 | 116 | 166 | 180 | 130 | 24 |
| B6 | 125 | 132 | 129 | 132 | 163 | 149 | 120 | 149 | 152 | 16 |
| B5 | 184 | 152 | 158 | 127 | 160 | 204 | 157 | 211 | 164 | 36 |
| B4 | 165 | 161 | 171 | 213 | 168 | 224 | 215 | 320 | 324 | 19 |
| B3 | 215 | 185 | 175 | 195 | 205 | 251 | 236 | 381 | 489 | 13 |
| B2 | 366 | 202 | 229 | 195 | 192 | 218 | 272 | 293 | 522 | 21 |
| B1 | 293 | 200 | 289 | 207 | 193 | 205 | 314 | 408 | 254 | 15 |
| B0 | 2590 | 1992 | 1998 | 1980 | 1892 | 2157 | 2652 | 2136 | 2172 | 39 |

TABLE 7
m1600 (3 d)

| | A0 | A1 | A2 | A3 | A4 | A5 | A6 | A7 | A8 | A9 |
|---|---|---|---|---|---|---|---|---|---|---|
| B9 | 903 | 752 | 962 | 655 | 916 | 1025 | 362 | 84 | 11 | 0 |
| B8 | 1378 | 681 | 1068 | 864 | 860 | 1201 | 710 | 506 | 1 | 3 |
| B7 | 1293 | 914 | 843 | 1125 | 949 | 1010 | 788 | 603 | 4 | 1 |
| B6 | 2324 | 1520 | 1322 | 1461 | 1377 | 1189 | 1084 | 776 | 16 | 1 |
| B5 | 3669 | 2665 | 2421 | 2385 | 2306 | 1815 | 1758 | 1316 | 39 | 0 |
| B4 | 4993 | 3411 | 3113 | 2983 | 3394 | 2569 | 2209 | 1469 | 155 | 2 |
| B3 | 5554 | 3343 | 3485 | 3461 | 3097 | 3047 | 3026 | 2491 | 149 | 1 |
| B2 | 3632 | 3511 | 3292 | 2799 | 3030 | 2658 | 3402 | 2449 | 348 | 1 |
| B1 | 4128 | 3619 | 3680 | 3292 | 2673 | 3558 | 3084 | 2515 | 279 | 1 |
| B0 | 4508 | 4146 | 4603 | 3714 | 3134 | 4045 | 4366 | 3730 | 487 | 4 |

TABLE 8
m877 (3 d)

| | A0 | A1 | A2 | A3 | A4 | A5 | A6 | A7 | A8 | A9 |
|---|---|---|---|---|---|---|---|---|---|---|
| B9 | 1493 | 1001 | 1072 | 939 | 1022 | 602 | 545 | 115 | 33 | 1 |
| B8 | 3082 | 2044 | 2043 | 1723 | 1443 | 1300 | 1062 | 887 | 120 | 1 |
| B7 | 3178 | 2006 | 2363 | 2011 | 1958 | 1806 | 1434 | 846 | 30 | 3 |
| B6 | 4243 | 3191 | 3536 | 3418 | 2958 | 2503 | 2316 | 1535 | 124 | 1 |
| B5 | 5835 | 4602 | 4504 | 4313 | 4520 | 4483 | 3710 | 1803 | 164 | 2 |
| B4 | 5764 | 5424 | 5650 | 5427 | 4935 | 4678 | 4574 | 2984 | 409 | 3 |
| B3 | 6331 | 5504 | 5305 | 5445 | 5331 | 5070 | 5369 | 3823 | 388 | 6 |
| B2 | 5629 | 5066 | 5289 | 5210 | 4956 | 5140 | 5345 | 4548 | 739 | 0 |
| B1 | 4914 | 4588 | 4458 | 4325 | 4972 | 5022 | 5021 | 4239 | 600 | 4 |
| B0 | 5076 | 5090 | 4877 | 4455 | 4789 | 5008 | 5125 | 4350 | 1205 | 2 |

TABLE 9
m3657 (7 d)

| | A0 | A1 | A2 | A3 | A4 | A5 | A6 | A7 | A8 | A9 |
|---|---|---|---|---|---|---|---|---|---|---|
| B9 | 40 | 28 | 50 | 54 | 31 | 26 | 22 | 37 | 20 | 18 |
| B8 | 158 | 178 | 171 | 189 | 197 | 143 | 72 | 34 | 38 | 19 |
| B7 | 229 | 210 | 214 | 239 | 223 | 185 | 112 | 39 | 37 | 3 |
| B6 | 293 | 298 | 295 | 347 | 322 | 250 | 118 | 53 | 39 | 13 |
| B5 | 433 | 385 | 524 | 439 | 437 | 374 | 211 | 86 | 37 | 16 |
| B4 | 1355 | 1081 | 1107 | 1194 | 1370 | 1056 | 882 | 199 | 48 | 15 |
| B3 | 1167 | 1389 | 1338 | 1314 | 1287 | 1074 | 800 | 338 | 73 | 14 |
| B2 | 1436 | 1334 | 1375 | 1157 | 1467 | 1102 | 1074 | 395 | 83 | 26 |
| B1 | 1326 | 1487 | 1245 | 1116 | 1115 | 1277 | 913 | 385 | 36 | 10 |
| B0 | 3780 | 3562 | 4473 | 4173 | 1159 | 3935 | 3317 | 4220 | 723 | 17 |

TABLE 10
m1338 (7 d)

| | A0 | A1 | A2 | A3 | A4 | A5 | A6 | A7 | A8 | A9 |
|---|---|---|---|---|---|---|---|---|---|---|
| B9 | 416 | 154 | 93 | 114 | 91 | 35 | 22 | 1 | 0 | 1 |
| B8 | 719 | 506 | 566 | 475 | 370 | 343 | 219 | 21 | 0 | 0 |
| B7 | 1374 | 825 | 640 | 590 | 674 | 594 | 292 | 57 | 0 | 0 |
| B6 | 1658 | 1073 | 825 | 883 | 781 | 724 | 431 | 114 | 3 | 0 |
| B5 | 1856 | 1407 | 1504 | 1426 | 1482 | 961 | 907 | 211 | 7 | 1 |
| B4 | 2628 | 2437 | 2519 | 2250 | 2363 | 2096 | 1537 | 668 | 27 | 0 |
| B3 | 3547 | 2493 | 2245 | 2641 | 2576 | 1893 | 2322 | 1565 | 85 | 0 |
| B2 | 2770 | 2366 | 2360 | 2735 | 2568 | 2327 | 2108 | 1704 | 222 | 0 |
| B1 | 3024 | 2426 | 2405 | 2281 | 2185 | 2636 | 2264 | 1410 | 171 | 1 |
| B0 | 3373 | 2207 | 3174 | 2916 | 2372 | 2788 | 2166 | 1468 | 292 | 1 |

TABLE 11
m4206 (3 d)

| | A0 | A1 | A2 | A3 | A4 | A5 | A6 | A7 | A8 | A9 |
|---|---|---|---|---|---|---|---|---|---|---|
| B9 | 1021 | 977 | 917 | 832 | 852 | 708 | 643 | 314 | 289 | 35 |
| B8 | 695 | 717 | 563 | 724 | 607 | 471 | 504 | 361 | 53 | 68 |
| B7 | 455 | 485 | 529 | 546 | 426 | 386 | 293 | 352 | 86 | 136 |
| B6 | 387 | 855 | 419 | 544 | 501 | 325 | 442 | 453 | 76 | 110 |
| B5 | 936 | 795 | 787 | 807 | 776 | 671 | 806 | 592 | 103 | 158 |
| B4 | 1460 | 1254 | 1439 | 1015 | 1115 | 1284 | 1069 | 866 | 108 | 157 |
| B3 | 1522 | 1967 | 1303 | 1395 | 1194 | 1189 | 1204 | 1066 | 81 | 223 |
| B2 | 1693 | 1681 | 1288 | 1313 | 1405 | 1141 | 1221 | 847 | 109 | 165 |
| B1 | 1859 | 1428 | 1318 | 1175 | 1552 | 996 | 1415 | 967 | 83 | 84 |
| B0 | 3409 | 3112 | 2484 | 3106 | 2323 | 2141 | 2304 | 1979 | 301 | 69 |

TABLE 12
m1717 (3 d)

| | A0 | A1 | A2 | A3 | A4 | A5 | A6 | A7 | A8 | A9 |
|---|---|---|---|---|---|---|---|---|---|---|
| B9 | 930 | 769 | 609 | 614 | 528 | 422 | 123 | 30 | 21 | 5 |
| B8 | 1991 | 1202 | 1127 | 1053 | 1309 | 1141 | 604 | 136 | 44 | 4 |
| B7 | 2044 | 1504 | 1314 | 1249 | 1388 | 1502 | 836 | 305 | 43 | 18 |
| B6 | 2596 | 1979 | 1651 | 1683 | 1793 | 1864 | 1330 | 716 | 28 | 10 |
| B5 | 3758 | 2853 | 2406 | 2418 | 2803 | 2826 | 2797 | 1036 | 28 | 6 |
| B4 | 6464 | 4162 | 3717 | 4089 | 4206 | 4587 | 3992 | 1813 | 104 | 15 |
| B3 | 6851 | 5534 | 4497 | 4211 | 4632 | 4665 | 4904 | 2817 | 146 | 3 |
| B2 | 7148 | 5854 | 5011 | 4310 | 5056 | 5180 | 4562 | 3396 | 309 | 17 |
| B1 | 6284 | 5714 | 4884 | 5097 | 4946 | 5285 | 5007 | 3081 | 278 | 13 |
| B0 | 6722 | 5977 | 6674 | 5155 | 6700 | 6589 | 5782 | 3762 | 571 | 24 |

TABLE 13
m3810 (3 d)

| | A0 | A1 | A2 | A3 | A4 | A5 | A6 | A7 | A8 | A9 |
|---|---|---|---|---|---|---|---|---|---|---|
| B9 | 74 | 106 | 74 | 101 | 79 | 113 | 77 | 30 | 7 | 10 |
| B8 | 113 | 164 | 141 | 163 | 144 | 184 | 167 | 102 | 16 | 11 |
| B7 | 130 | 139 | 143 | 178 | 136 | 144 | 139 | 134 | 14 | 10 |
| B6 | 111 | 185 | 158 | 199 | 157 | 185 | 165 | 175 | 23 | 17 |
| B5 | 265 | 295 | 302 | 287 | 295 | 262 | 268 | 235 | 60 | 7 |
| B4 | 613 | 757 | 612 | 596 | 530 | 470 | 404 | 358 | 87 | 11 |
| B3 | 597 | 837 | 764 | 780 | 627 | 566 | 486 | 405 | 85 | 6 |
| B2 | 727 | 763 | 701 | 676 | 647 | 478 | 482 | 385 | 139 | 15 |
| B1 | 582 | 530 | 552 | 556 | 613 | 663 | 512 | 498 | 104 | 16 |
| B0 | 2123 | 1232 | 2209 | 1405 | 1982 | 1472 | 1497 | 1054 | 268 | 8 |

TABLE 14
m4206 (7 d)

| | A0 | A1 | A2 | A3 | A4 | A5 | A6 | A7 | A8 | A9 |
|---|---|---|---|---|---|---|---|---|---|---|
| B9 | 60 | 83 | 120 | 121 | 214 | 508 | 1228 | 1264 | 1368 | 4881 |
| B8 | 91 | 65 | 108 | 92 | 156 | 663 | 1243 | 1047 | 1229 | 3666 |
| B7 | 84 | 93 | 70 | 103 | 204 | 602 | 1188 | 1369 | 1355 | 4685 |
| B6 | 70 | 60 | 82 | 118 | 111 | 728 | 1150 | 1110 | 923 | 3684 |
| B5 | 71 | 53 | 108 | 76 | 196 | 429 | 1285 | 1421 | 1180 | 3902 |
| B4 | 58 | 80 | 91 | 112 | 137 | 501 | 927 | 1090 | 942 | 3737 |
| B3 | 58 | 73 | 111 | 126 | 91 | 295 | 720 | 844 | 688 | 3282 |
| B2 | 147 | 129 | 98 | 115 | 164 | 131 | 306 | 309 | 245 | 1532 |
| B1 | 161 | 74 | 181 | 86 | 222 | 220 | 308 | 258 | 241 | 143 |
| B0 | 32 | 56 | 52 | 86 | 37 | 83 | 87 | 68 | 24 | 35 |

TABLE 15
X-SK-5864D (7 d)

| | A0 | A1 | A2 | A3 | A4 | A5 | A6 | A7 | A8 | A9 |
|---|---|---|---|---|---|---|---|---|---|---|
| B9 | 51 | 99 | 75 | 55 | 75 | 47 | 43 | 37 | 24 | 19 |
| B8 | 65 | 114 | 137 | 138 | 122 | 94 | 87 | 60 | 41 | 7 |
| B7 | 158 | 177 | 148 | 152 | 196 | 117 | 93 | 88 | 49 | 37 |
| B6 | 189 | 156 | 250 | 273 | 158 | 159 | 129 | 122 | 47 | 93 |
| B5 | 402 | 398 | 390 | 410 | 338 | 332 | 343 | 184 | 36 | 114 |
| B4 | 2636 | 1761 | 1791 | 1219 | 1433 | 1417 | 1291 | 591 | 86 | 114 |
| B3 | 3379 | 4002 | 3867 | 3538 | 3231 | 3073 | 3105 | 2044 | 176 | 103 |
| B2 | 4746 | 3760 | 3869 | 3618 | 3415 | 3979 | 1782 | 2008 | 267 | 72 |
| B1 | 4600 | 4300 | 4111 | 3727 | 4171 | 3638 | 2151 | 1541 | 251 | 51 |
| B0 | 8994 | 8655 | 8407 | 8740 | 9490 | 9099 | 8295 | 6625 | 1834 | 39 |

TABLE 16
m1560 (3 d)

| | A0 | A1 | A2 | A3 | A4 | A5 | A6 | A7 | A8 | A9 |
|---|---|---|---|---|---|---|---|---|---|---|
| B9 | 119 | 143 | 155 | 113 | 150 | 141 | 154 | 85 | 85 | 11 |
| B8 | 978 | 940 | 1061 | 886 | 776 | 753 | 679 | 340 | 99 | 18 |
| B7 | 1615 | 1448 | 1443 | 1600 | 1556 | 1230 | 1033 | 549 | 102 | 21 |
| B6 | 2147 | 1676 | 2022 | 2336 | 1966 | 1799 | 1513 | 933 | 210 | 8 |
| B5 | 3294 | 3216 | 3318 | 3021 | 3440 | 3225 | 2479 | 1175 | 233 | 18 |
| B4 | 3833 | 4207 | 3976 | 3657 | 3629 | 3192 | 2661 | 1564 | 848 | 7 |
| B3 | 4034 | 3754 | 4065 | 4060 | 4553 | 3549 | 3858 | 3156 | 912 | 9 |
| B2 | 4644 | 3954 | 4070 | 4143 | 4767 | 3794 | 3995 | 3859 | 1144 | 19 |
| B1 | 4009 | 3477 | 3926 | 3794 | 4154 | 3510 | 4199 | 3495 | 979 | 12 |
| B0 | 3868 | 3831 | 4353 | 3990 | 4102 | 4790 | 4389 | 4671 | 1355 | 11 |

TABLE 17
XX-SK-15723 (3 d)

| | A0 | A1 | A2 | A3 | A4 | A5 | A6 | A7 | A8 | A9 |
|---|---|---|---|---|---|---|---|---|---|---|
| B9 | 127 | 164 | 148 | 185 | 154 | 146 | 129 | 68 | 116 | 251 |
| B8 | 219 | 252 | 148 | 316 | 357 | 185 | 181 | 119 | 101 | 353 |
| B7 | 358 | 290 | 248 | 331 | 325 | 281 | 243 | 174 | 105 | 455 |
| B6 | 440 | 391 | 353 | 309 | 440 | 298 | 275 | 219 | 123 | 571 |
| B5 | 574 | 500 | 642 | 561 | 786 | 533 | 618 | 367 | 189 | 552 |
| B4 | 807 | 845 | 688 | 888 | 834 | 747 | 822 | 521 | 151 | 658 |
| B3 | 882 | 944 | 983 | 973 | 951 | 1114 | 961 | 718 | 181 | 421 |
| B2 | 847 | 863 | 821 | 748 | 900 | 1008 | 771 | 632 | 165 | 503 |
| B1 | 875 | 894 | 915 | 723 | 811 | 808 | 909 | 766 | 155 | 548 |
| B0 | 1151 | 1111 | 1324 | 1511 | 1030 | 1379 | 1698 | 1672 | 381 | 465 |

TABLE 18
X-SK-14316 (3 d)

| | A0 | A1 | A2 | A3 | A4 | A5 | A6 | A7 | A8 | A9 |
|---|---|---|---|---|---|---|---|---|---|---|
| B9 | 102 | 148 | 78 | 65 | 69 | 58 | 67 | 17 | 21 | 7 |
| B8 | 191 | 116 | 100 | 110 | 73 | 50 | 49 | 14 | 11 | 5 |
| B7 | 140 | 171 | 164 | 147 | 156 | 139 | 70 | 15 | 20 | 2 |
| B6 | 279 | 230 | 229 | 202 | 181 | 235 | 135 | 69 | 14 | 5 |
| B5 | 311 | 292 | 338 | 318 | 375 | 284 | 299 | 218 | 33 | 7 |
| B4 | 603 | 373 | 415 | 426 | 436 | 416 | 463 | 337 | 131 | 13 |
| B3 | 516 | 453 | 457 | 596 | 595 | 560 | 612 | 460 | 215 | 4 |
| B2 | 617 | 423 | 515 | 432 | 459 | 410 | 522 | 467 | 278 | 1 |
| B1 | 684 | 570 | 488 | 502 | 561 | 463 | 503 | 460 | 198 | 1 |
| B0 | 1067 | 825 | 708 | 984 | 778 | 710 | 727 | 584 | 345 | 2 |

TABLE 19
m3596 (3 d)

| | A0 | A1 | A2 | A3 | A4 | A5 | A6 | A7 | A8 | A9 |
|---|---|---|---|---|---|---|---|---|---|---|
| B9 | 658 | 682 | 757 | 545 | 767 | 658 | 278 | 82 | 34 | 10 |
| B8 | 1101 | 1035 | 1472 | 917 | 1258 | 743 | 926 | 448 | 131 | 39 |
| B7 | 1079 | 1248 | 1240 | 1111 | 1215 | 1313 | 924 | 622 | 111 | 25 |
| B6 | 1344 | 1196 | 1412 | 1309 | 1014 | 1161 | 1149 | 648 | 64 | 11 |
| B5 | 1853 | 1900 | 2016 | 1756 | 1947 | 1385 | 1129 | 658 | 67 | 16 |
| B4 | 3093 | 3011 | 3067 | 2970 | 2953 | 2790 | 2388 | 1594 | 136 | 16 |
| B3 | 3031 | 2989 | 2977 | 3050 | 2966 | 2368 | 2683 | 1829 | 165 | 25 |
| B2 | 3392 | 3588 | 2861 | 3154 | 3237 | 2842 | 2462 | 1768 | 103 | 21 |
| B1 | 3162 | 3260 | 3091 | 3260 | 2900 | 2698 | 2869 | 1414 | 137 | 22 |
| B0 | 5541 | 5112 | 5583 | 5099 | 4098 | 5124 | 4525 | 3446 | 178 | 14 |

TABLE 20
XX-IV-182 (3 d)

| | A0 | A1 | A2 | A3 | A4 | A5 | A6 | A7 | A8 | A9 |
|---|---|---|---|---|---|---|---|---|---|---|
| B9 | 999 | 897 | 955 | 924 | 876 | 851 | 1022 | 480 | 27 | 14 |
| B8 | 1497 | 1680 | 1723 | 1492 | 1717 | 1430 | 1533 | 966 | 93 | 9 |
| B7 | 2067 | 1897 | 1727 | 1697 | 1861 | 1679 | 1579 | 1069 | 65 | 16 |
| B6 | 2253 | 2501 | 1925 | 1545 | 1702 | 1701 | 1818 | 1152 | 203 | 19 |
| B5 | 2324 | 2869 | 2703 | 2308 | 2611 | 2573 | 2328 | 1139 | 248 | 2 |
| B4 | 3138 | 3353 | 3121 | 3150 | 3167 | 2990 | 2731 | 2323 | 649 | 3 |
| B3 | 2857 | 2994 | 3325 | 3426 | 3359 | 3282 | 3013 | 2810 | 1228 | 19 |
| B2 | 3257 | 3303 | 3281 | 3127 | 3098 | 3480 | 2446 | 2845 | 1612 | 18 |
| B1 | 3169 | 3025 | 3402 | 3353 | 2759 | 2989 | 2927 | 2748 | 1302 | 10 |
| B0 | 3226 | 3476 | 3328 | 3487 | 3721 | 3248 | 3111 | 2076 | 1869 | 0 |

TABLE 21
m1594 (3 d)

| | A0 | A1 | A2 | A3 | A4 | A5 | A6 | A7 | A8 | A9 |
|---|---|---|---|---|---|---|---|---|---|---|
| B9 | 865 | 442 | 451 | 383 | 512 | 391 | 139 | 88 | 17 | 17 |
| B8 | 1142 | 576 | 462 | 534 | 691 | 524 | 324 | 165 | 136 | 5 |
| B7 | 1253 | 672 | 763 | 649 | 641 | 671 | 453 | 196 | 124 | 16 |
| B6 | 1692 | 1363 | 1093 | 1111 | 995 | 1015 | 790 | 333 | 103 | 11 |
| B5 | 2745 | 2640 | 2671 | 2444 | 2012 | 2458 | 1929 | 929 | 249 | 23 |
| B4 | 4818 | 3651 | 4455 | 4229 | 3751 | 3578 | 4408 | 2798 | 230 | 29 |
| B3 | 4966 | 5215 | 4568 | 3850 | 4854 | 5223 | 4688 | 3445 | 492 | 41 |
| B2 | 5720 | 4808 | 1079 | 4927 | 4907 | 4909 | 4890 | 3847 | 697 | 70 |
| B1 | 5417 | 4645 | 4754 | 5119 | 4584 | 4351 | 5212 | 3919 | 533 | 24 |
| B0 | 7645 | 6401 | 5827 | 5690 | 5212 | 5817 | 6001 | 5577 | 1349 | 21 |

TABLE 22
XX-SK-15723 (3 d)

| | A0 | A1 | A2 | A3 | A4 | A5 | A6 | A7 | A8 | A9 |
|---|---|---|---|---|---|---|---|---|---|---|
| B9 | 127 | 164 | 148 | 185 | 154 | 146 | 129 | 68 | 116 | 251 |
| B8 | 219 | 252 | 148 | 316 | 357 | 185 | 181 | 119 | 101 | 353 |
| B7 | 358 | 290 | 248 | 331 | 325 | 281 | 243 | 174 | 105 | 455 |
| B6 | 440 | 391 | 353 | 309 | 440 | 298 | 275 | 219 | 123 | 571 |
| B5 | 574 | 500 | 642 | 561 | 786 | 533 | 618 | 367 | 189 | 552 |
| B4 | 807 | 845 | 688 | 888 | 834 | 747 | 822 | 521 | 151 | 658 |
| B3 | 882 | 944 | 983 | 973 | 951 | 1114 | 961 | 718 | 181 | 421 |
| B2 | 847 | 863 | 821 | 748 | 900 | 1008 | 771 | 632 | 165 | 503 |
| B1 | 875 | 894 | 915 | 723 | 811 | 808 | 909 | 766 | 155 | 548 |
| B0 | 1151 | 1111 | 1324 | 1511 | 1030 | 1379 | 1698 | 1672 | 381 | 465 |

TABLE 23
X-SK-6731 (3 d)

| | A0 | A1 | A2 | A3 | A4 | A5 | A6 | A7 | A8 | A9 |
|---|---|---|---|---|---|---|---|---|---|---|
| B9 | 133 | 47 | 48 | 44 | 87 | 35 | 47 | 1 | 0 | 0 |
| B8 | 335 | 200 | 164 | 122 | 170 | 156 | 174 | 50 | 0 | 0 |
| B7 | 362 | 175 | 151 | 134 | 193 | 149 | 165 | 80 | 0 | 0 |
| B6 | 549 | 385 | 235 | 258 | 310 | 273 | 262 | 110 | 4 | 0 |
| B5 | 1553 | 826 | 723 | 672 | 790 | 721 | 650 | 160 | 0 | 0 |
| B4 | 3585 | 2061 | 1645 | 1597 | 1448 | 1532 | 1388 | 554 | 23 | 0 |
| B3 | 3856 | 2579 | 2112 | 2186 | 1870 | 1821 | 1427 | 652 | 16 | 0 |
| B2 | 3698 | 2289 | 2404 | 1793 | 1991 | 1715 | 1767 | 818 | 76 | 0 |
| B1 | 3499 | 2563 | 2168 | 2073 | 2006 | 1830 | 1827 | 858 | 58 | 0 |
| B0 | 4324 | 3005 | 2586 | 2601 | 2507 | 2249 | 2333 | 1142 | 160 | 0 |

TABLE 24
m3596 (7 d)

| | A0 | A1 | A2 | A3 | A4 | A5 | A6 | A7 | A8 | A9 |
|---|---|---|---|---|---|---|---|---|---|---|
| B9 | 135 | 75 | 124 | 85 | 60 | 41 | 50 | 15 | 3 | 0 |
| B8 | 265 | 289 | 115 | 324 | 221 | 383 | 355 | 100 | 3 | 1 |
| B7 | 273 | 304 | 172 | 186 | 346 | 297 | 173 | 165 | 2 | 4 |
| B6 | 325 | 345 | 297 | 379 | 340 | 383 | 387 | 117 | 21 | 0 |
| B5 | 125 | 369 | 300 | 365 | 541 | 514 | 511 | 241 | 8 | 2 |
| B4 | 933 | 1003 | 950 | 707 | 917 | 1034 | 1075 | 645 | 38 | 0 |
| B3 | 1423 | 1544 | 1380 | 1861 | 1551 | 1880 | 1018 | 864 | 58 | 0 |
| B2 | 1727 | 1460 | 1675 | 1459 | 1476 | 1610 | 1647 | 943 | 45 | 0 |
| B1 | 1727 | 1492 | 1496 | 1719 | 1652 | 1240 | 1577 | 1046 | 33 | 0 |
| B0 | 3310 | 3788 | 4101 | 3585 | 3908 | 3717 | 3560 | 2162 | 94 | 1 |

TABLE 25
m3657 (3 d)

| | A0 | A1 | A2 | A3 | A4 | A5 | A6 | A7 | A8 | A9 |
|---|---|---|---|---|---|---|---|---|---|---|
| B9 | 181 | 134 | 192 | 218 | 160 | 127 | 70 | 12 | 8 | 3 |
| B8 | 420 | 388 | 377 | 369 | 394 | 214 | 166 | 47 | 11 | 0 |
| B7 | 423 | 403 | 515 | 478 | 456 | 639 | 207 | 103 | 35 | 2 |
| B6 | 568 | 476 | 581 | 603 | 506 | 486 | 275 | 113 | 47 | 1 |
| B5 | 681 | 790 | 726 | 661 | 937 | 527 | 488 | 183 | 79 | 3 |
| B4 | 1208 | 1076 | 1353 | 1081 | 1570 | 1326 | 926 | 558 | 161 | 2 |
| B3 | 1194 | 1328 | 1385 | 1408 | 1317 | 1108 | 1337 | 729 | 214 | 0 |
| B2 | 1311 | 996 | 1404 | 1339 | 1498 | 1325 | 1385 | 919 | 223 | 3 |
| B1 | 1345 | 1538 | 1260 | 1552 | 1246 | 1210 | 1436 | 749 | 228 | 2 |
| B0 | 1632 | 1929 | 2171 | 2106 | 2057 | 2138 | 2291 | 1780 | 1080 | 2 |

TABLE 26
m1307 (3 d)

| | A0 | A1 | A2 | A3 | A4 | A5 | A6 | A7 | A8 | A9 |
|---|---|---|---|---|---|---|---|---|---|---|
| B9 | 388 | 386 | 0 | 342 | 321 | 239 | 120 | 46 | 6 | 9 |
| B8 | 534 | 638 | 0 | 549 | 505 | 434 | 241 | 41 | 106 | 0 |
| B7 | 960 | 997 | 965 | 1063 | 900 | 695 | 385 | 101 | 209 | 2 |
| B6 | 1452 | 1491 | 1437 | 1433 | 1302 | 1090 | 528 | 74 | 367 | 4 |
| B5 | 3518 | 3168 | 3282 | 3438 | 3088 | 1227 | 2237 | 417 | 506 | 7 |
| B4 | 5749 | 5024 | 6133 | 5253 | 5504 | 5263 | 4846 | 2818 | 697 | 6 |
| B3 | 4119 | 5503 | 6015 | 5491 | 5466 | 5547 | 5522 | 3270 | 1570 | 1 |
| B2 | 5036 | 5245 | 5008 | 5320 | 5112 | 4896 | 4325 | 3420 | 1513 | 5 |
| B1 | 5093 | 5163 | 1240 | 5415 | 5186 | 5088 | 5274 | 3445 | 1610 | 5 |
| B0 | 5757 | 6152 | 6130 | 6246 | 5821 | 5979 | 6580 | 6173 | 3087 | 3 |

TABLE 27
m1307 (7 d)

| | A0 | A1 | A2 | A3 | A4 | A5 | A6 | A7 | A8 | A9 |
|---|---|---|---|---|---|---|---|---|---|---|
| B9 | 31 | 25 | 50 | 38 | 45 | 44 | 26 | 11 | 0 | 1 |
| B8 | 105 | 167 | 99 | 130 | 167 | 83 | 55 | 40 | 41 | 0 |
| B7 | 295 | 224 | 225 | 359 | 264 | 149 | 310 | 35 | 58 | 0 |
| B6 | 409 | 406 | 459 | 476 | 441 | 26 | 83 | 70 | 55 | 1 |
| B5 | 729 | 1441 | 1401 | 1564 | 1537 | 614 | 573 | 140 | 99 | 0 |
| B4 | 3714 | 3643 | 3874 | 2696 | 4135 | 3031 | 2413 | 846 | 112 | 0 |
| B3 | 4767 | 5023 | 5325 | 4275 | 4510 | 4317 | 3435 | 1691 | 187 | 0 |
| B2 | 3973 | 4581 | 5143 | 4683 | 4904 | 4696 | 3887 | 2438 | 411 | 1 |
| B1 | 4509 | 4912 | 4491 | 4869 | 4162 | 4063 | 3458 | 2487 | 293 | 0 |
| B0 | 6796 | 7142 | 7897 | 6024 | 7716 | 7448 | 6827 | 6821 | 3341 | 0 |

TABLE 28
m4138 (7 d)

| | A0 | A1 | A2 | A3 | A4 | A5 | A6 | A7 | A8 | A9 |
|---|---|---|---|---|---|---|---|---|---|---|
| B9 | 56 | 65 | 51 | 72 | 54 | 45 | 41 | 91 | 121 | 144 |
| B8 | 63 | 54 | 74 | 76 | 67 | 50 | 47 | 99 | 109 | 161 |
| B7 | 90 | 58 | 91 | 42 | 101 | 53 | 36 | 64 | 92 | 210 |
| B6 | 69 | 110 | 53 | 95 | 77 | 94 | 74 | 54 | 40 | 168 |
| B5 | 174 | 111 | 150 | 134 | 101 | 98 | 107 | 66 | 63 | 137 |
| B4 | 467 | 230 | 397 | 365 | 332 | 271 | 116 | 104 | 28 | 114 |
| B3 | 1115 | 386 | 1185 | 1033 | 975 | 1015 | 929 | 108 | 110 | 172 |
| B2 | 1232 | 1233 | 1406 | 1414 | 479 | 1319 | 632 | 294 | 55 | 135 |
| B1 | 804 | 1419 | 1197 | 1277 | 1425 | 677 | 1114 | 114 | 86 | 69 |
| B0 | 1058 | 1607 | 2092 | 2136 | 1740 | 664 | 1893 | 865 | 128 | 97 |

TABLE 29
m1106 (3 d)

| | A0 | A1 | A2 | A3 | A4 | A5 | A6 | A7 | A8 | A9 |
|---|---|---|---|---|---|---|---|---|---|---|
| B9 | 650 | 646 | 546 | 741 | 959 | 1193 | 2189 | 1863 | 3 | 12 |
| B8 | 1731 | 1724 | 1447 | 1244 | 1545 | 1473 | 1887 | 1369 | 793 | 31 |
| B7 | 2418 | 2400 | 2252 | 2591 | 2682 | 2315 | 2209 | 1392 | 623 | 48 |
| B6 | 2654 | 2651 | 2553 | 2732 | 2531 | 2543 | 2651 | 1944 | 562 | 47 |
| B5 | 3473 | 3349 | 3123 | 2866 | 3057 | 2720 | 3629 | 2572 | 481 | 49 |
| B4 | 3863 | 3955 | 3831 | 3951 | 4091 | 3768 | 4178 | 2661 | 519 | 47 |
| B3 | 4387 | 4626 | 4713 | 4504 | 4405 | 4092 | 3979 | 3063 | 562 | 47 |
| B2 | 4081 | 4611 | 4537 | 4426 | 4477 | 4039 | 4568 | 2968 | 1117 | 34 |
| B1 | 4532 | 4585 | 4829 | 4710 | 4765 | 4348 | 4486 | 2908 | 626 | 49 |
| B0 | 5947 | 7059 | 6717 | 7505 | 7175 | 6517 | 6405 | 4699 | 1192 | 40 |

TABLE 30
m1371 (7 d)

| | A0 | A1 | A2 | A3 | A4 | A5 | A6 | A7 | A8 | A9 |
|---|---|---|---|---|---|---|---|---|---|---|
| B9 | 194 | 234 | 219 | 184 | 226 | 177 | 105 | 55 | 10 | 10 |
| B8 | 384 | 371 | 372 | 341 | 333 | 353 | 347 | 258 | 24 | 11 |
| B7 | 441 | 465 | 346 | 431 | 417 | 421 | 429 | 312 | 28 | 16 |
| B6 | 571 | 515 | 564 | 589 | 531 | 582 | 539 | 424 | 40 | 7 |
| B5 | 778 | 928 | 772 | 745 | 810 | 725 | 820 | 637 | 71 | 9 |
| B4 | 1139 | 1278 | 1054 | 979 | 1018 | 1109 | 1308 | 996 | 73 | 7 |
| B3 | 1046 | 1071 | 1074 | 1160 | 1251 | 722 | 1184 | 1046 | 461 | 2 |
| B2 | 990 | 1044 | 1094 | 1133 | 1361 | 1049 | 1501 | 1065 | 169 | 4 |
| B1 | 1099 | 1093 | 121 | 1071 | 1189 | 1223 | 1438 | 897 | 113 | 3 |
| B0 | 1382 | 1469 | 1056 | 1428 | 1411 | 1380 | 1399 | 1172 | 205 | 9 |

TABLE 31
m4138 (3 d)

| | A0 | A1 | A2 | A3 | A4 | A5 | A6 | A7 | A8 | A9 |
|---|---|---|---|---|---|---|---|---|---|---|
| B9 | 176 | 272 | 176 | 228 | 224 | 212 | 187 | 63 | 76 | 5 |
| B8 | 193 | 214 | 220 | 231 | 203 | 215 | 173 | 111 | 36 | 10 |
| B7 | 266 | 251 | 275 | 236 | 219 | 219 | 247 | 105 | 71 | 5 |
| B6 | 279 | 239 | 298 | 276 | 318 | 304 | 277 | 129 | 64 | 10 |
| B5 | 605 | 683 | 625 | 596 | 699 | 673 | 501 | 135 | 74 | 7 |
| B4 | 1497 | 1661 | 1509 | 1499 | 1533 | 750 | 1257 | 577 | 84 | 5 |
| B3 | 1881 | 2305 | 2198 | 1181 | 2342 | 2103 | 1544 | 551 | 45 | 8 |
| B2 | 2177 | 1676 | 1440 | 2557 | 2302 | 2100 | 756 | 601 | 66 | 17 |
| B1 | 2188 | 2236 | 2127 | 1913 | 2477 | 1903 | 1610 | 542 | 68 | 7 |
| B0 | 3174 | 3250 | 3053 | 3433 | 3000 | 3209 | 2230 | 1278 | 108 | 7 |

TABLE 32
m3320 (3 d)

| | A0 | A1 | A2 | A3 | A4 | A5 | A6 | A7 | A8 | A9 |
|---|---|---|---|---|---|---|---|---|---|---|
| B9 | 15 | 16 | 19 | 27 | 20 | 24 | 14 | 9 | 5 | 3 |
| B8 | 24 | 26 | 17 | 33 | 23 | 33 | 20 | 8 | 1 | 0 |
| B7 | 30 | 37 | 32 | 46 | 37 | 14 | 25 | 12 | 3 | 0 |
| B6 | 63 | 52 | 48 | 81 | 78 | 54 | 40 | 13 | 9 | 1 |
| B5 | 515 | 624 | 511 | 431 | 493 | 575 | 451 | 90 | 12 | 2 |
| B4 | 3239 | 2531 | 2320 | 2283 | 2228 | 2322 | 1999 | 1351 | 73 | 1 |
| B3 | 3272 | 2712 | 2588 | 2856 | 2542 | 2648 | 3112 | 2249 | 90 | 0 |
| B2 | 3622 | 2560 | 2755 | 2800 | 2646 | 2769 | 3104 | 2355 | 150 | 2 |
| B1 | 3835 | 3190 | 2471 | 2690 | 2573 | 2501 | 3137 | 2542 | 175 | 0 |
| B0 | 11445 | 8255 | 8872 | 9248 | 9768 | 9770 | 10719 | 4017 | 268 | 1 |

TABLE 33
XX-IV-113 (7 d)

| | A0 | A1 | A2 | A3 | A4 | A5 | A6 | A7 | A8 | A9 |
|---|---|---|---|---|---|---|---|---|---|---|
| B9 | 17 | 18 | 10 | 14 | 9 | 19 | 12 | 21 | 14 | 12 |
| B8 | 47 | 36 | 52 | 30 | 36 | 15 | 26 | 22 | 5 | 16 |
| B7 | 151 | 105 | 101 | 91 | 109 | 87 | 57 | 29 | 29 | 0 |
| B6 | 118 | 119 | 137 | 109 | 99 | 70 | 66 | 62 | 46 | 24 |
| B5 | 289 | 113 | 207 | 146 | 117 | 177 | 142 | 121 | 34 | 62 |
| B4 | 11422 | 1623 | 1975 | 12276 | 12872 | 4413 | 11328 | 1129 | 870 | 67 |
| B3 | 12123 | 11710 | 8351 | 12522 | 12973 | 13308 | 12790 | 10315 | 1345 | 53 |
| B2 | 12532 | 9461 | 12821 | 14151 | 13255 | 12266 | 12504 | 10300 | 1807 | 31 |
| B1 | 13471 | 10837 | 12433 | 3253 | 11829 | 13564 | 13778 | 9590 | 1622 | 63 |
| B0 | 11196 | 13403 | 13329 | 13281 | 12393 | 13143 | 13327 | 9922 | 2073 | 83 |

TABLE 34
m3794 (3 d)

| | A0 | A1 | A2 | A3 | A4 | A5 | A6 | A7 | A8 | A9 |
|---|---|---|---|---|---|---|---|---|---|---|
| B9 | 212 | 216 | 162 | 131 | 132 | 149 | 51 | 37 | 93 | 28 |
| B8 | 402 | 420 | 271 | 295 | 408 | 334 | 197 | 88 | 52 | 42 |
| B7 | 364 | 341 | 334 | 401 | 319 | 291 | 185 | 64 | 49 | 47 |
| B6 | 482 | 507 | 560 | 326 | 504 | 458 | 262 | 98 | 76 | 23 |
| B5 | 923 | 1120 | 1013 | 1391 | 1093 | 982 | 670 | 175 | 52 | 50 |
| B4 | 2005 | 2608 | 2312 | 2000 | 1860 | 2214 | 1942 | 977 | 84 | 16 |
| B3 | 1841 | 2123 | 2555 | 2387 | 2294 | 2372 | 2374 | 1700 | 99 | 30 |
| B2 | 2257 | 2301 | 2703 | 2356 | 2362 | 2093 | 2395 | 1637 | 165 | 20 |
| B1 | 2665 | 2690 | 2752 | 2270 | 2289 | 2193 | 2524 | 1492 | 148 | 37 |
| B0 | 2813 | 2473 | 2024 | 2610 | 2444 | 2859 | 2760 | 2195 | 254 | 28 |

TABLE 35
m3147 (7 d)

| | A0 | A1 | A2 | A3 | A4 | A5 | A6 | A7 | A8 | A9 |
|---|---|---|---|---|---|---|---|---|---|---|
| B9 | 34 | 53 | 21 | 30 | 55 | 18 | 18 | 8 | 3 | 10 |
| B8 | 247 | 234 | 229 | 304 | 195 | 267 | 178 | 103 | 29 | 4 |
| B7 | 294 | 456 | 209 | 406 | 408 | 303 | 274 | 161 | 32 | 0 |
| B6 | 548 | 386 | 440 | 371 | 444 | 514 | 409 | 266 | 27 | 1 |
| B5 | 836 | 803 | 784 | 730 | 975 | 829 | 748 | 456 | 74 | 3 |
| B4 | 1534 | 1494 | 1492 | 1511 | 1072 | 1199 | 1283 | 1008 | 289 | 4 |
| B3 | 2244 | 1912 | 1979 | 1913 | 2093 | 1971 | 2063 | 1290 | 647 | 3 |
| B2 | 1812 | 1663 | 1983 | 1883 | 2046 | 1993 | 1785 | 1590 | 745 | 4 |
| B1 | 1731 | 1808 | 1968 | 1737 | 2138 | 1467 | 2102 | 1984 | 741 | 2 |
| B0 | 2298 | 2778 | 3241 | 2785 | 2761 | 2128 | 2825 | 2125 | 1167 | 4 |

TABLE 36
m1594 (7 d)

| | A0 | A1 | A2 | A3 | A4 | A5 | A6 | A7 | A8 | A9 |
|---|---|---|---|---|---|---|---|---|---|---|
| B9 | 25 | 21 | 10 | 18 | 25 | 21 | 40 | 20 | 5 | 1 |
| B8 | 338 | 54 | 25 | 31 | 28 | 24 | 24 | 19 | 49 | 4 |
| B7 | 550 | 92 | 49 | 34 | 47 | 22 | 58 | 20 | 8 | 1 |
| B6 | 742 | 192 | 128 | 77 | 69 | 74 | 38 | 33 | 10 | 4 |
| B5 | 2020 | 588 | 454 | 321 | 277 | 209 | 241 | 119 | 25 | 29 |
| B4 | 3788 | 1571 | 1660 | 1212 | 1270 | 1080 | 1147 | 420 | 45 | 33 |
| B3 | 4466 | 2702 | 2772 | 2471 | 2244 | 2276 | 1863 | 1178 | 99 | 15 |
| B2 | 4888 | 3427 | 3001 | 2499 | 2276 | 1849 | 2480 | 1431 | 168 | 46 |
| B1 | 5789 | 2679 | 2665 | 2247 | 2219 | 2406 | 2568 | 1518 | 151 | 9 |
| B0 | 8723 | 5777 | 4973 | 4136 | 3129 | 4102 | 3755 | 3900 | 725 | 20 |

TABLE 37
m3794 (7 d)

| | A0 | A1 | A2 | A3 | A4 | A5 | A6 | A7 | A8 | A9 |
|---|---|---|---|---|---|---|---|---|---|---|
| B9 | 9 | 10 | 14 | 2 | 5 | 7 | 6 | 4 | 8 | 7 |
| B8 | 36 | 37 | 41 | 37 | 64 | 37 | 12 | 8 | 5 | 8 |
| B7 | 88 | 68 | 43 | 79 | 45 | 51 | 51 | 8 | 2 | 35 |
| B6 | 126 | 143 | 138 | 137 | 124 | 119 | 62 | 15 | 9 | 17 |
| B5 | 467 | 499 | 523 | 398 | 496 | 391 | 200 | 41 | 12 | 35 |
| B4 | 1208 | 1507 | 1452 | 1448 | 1162 | 1352 | 1108 | 272 | 13 | 43 |
| B3 | 1801 | 2029 | 1852 | 2211 | 2178 | 2226 | 1646 | 819 | 15 | 13 |
| B2 | 2239 | 2274 | 2070 | 2253 | 1826 | 2104 | 2044 | 939 | 18 | 12 |
| B1 | 1864 | 2471 | 2097 | 2481 | 2411 | 2192 | 1655 | 905 | 16 | 8 |
| B0 | 4936 | 3956 | 4074 | 4998 | 3613 | 4348 | 3709 | 2630 | 271 | 26 |

TABLE 38
m3153 (7 d)

| | A0 | A1 | A2 | A3 | A4 | A5 | A6 | A7 | A8 | A9 |
|---|---|---|---|---|---|---|---|---|---|---|
| B9 | 46 | 21 | 10 | 17 | 10 | 28 | 38 | 15 | 13 | 13 |
| B8 | 132 | 107 | 85 | 122 | 84 | 124 | 116 | 61 | 15 | 28 |
| B7 | 178 | 212 | 202 | 241 | 237 | 270 | 171 | 133 | 15 | 28 |
| B6 | 334 | 318 | 290 | 231 | 323 | 358 | 378 | 181 | 24 | 32 |
| B5 | 550 | 464 | 450 | 296 | 499 | 507 | 632 | 336 | 27 | 18 |
| B4 | 1178 | 1208 | 1194 | 1094 | 1311 | 1255 | 1257 | 976 | 73 | 19 |
| B3 | 1296 | 1262 | 1292 | 1430 | 1444 | 1466 | 1488 | 1208 | 101 | 16 |
| B2 | 1202 | 1170 | 1303 | 1440 | 1349 | 1395 | 1403 | 1176 | 183 | 17 |
| B1 | 1122 | 1272 | 1413 | 1242 | 1393 | 1412 | 1483 | 1096 | 115 | 31 |
| B0 | 2081 | 2288 | 2291 | 2505 | 2446 | 1542 | 2303 | 2087 | 545 | 73 |

TABLE 39
m1560 (7 d)

| | A0 | A1 | A2 | A3 | A4 | A5 | A6 | A7 | A8 | A9 |
|---|---|---|---|---|---|---|---|---|---|---|
| B9 | 10 | 6 | 8 | 13 | 16 | 9 | 6 | 9 | 18 | 3 |
| B8 | 128 | 135 | 137 | 113 | 126 | 64 | 45 | 16 | 32 | 2 |
| B7 | 370 | 334 | 306 | 288 | 280 | 218 | 222 | 72 | 30 | 0 |
| B6 | 342 | 669 | 479 | 664 | 657 | 628 | 447 | 171 | 42 | 29 |
| B5 | 1595 | 1420 | 1542 | 1620 | 1729 | 1781 | 1241 | 435 | 101 | 7 |
| B4 | 3358 | 3091 | 3094 | 3145 | 2911 | 3093 | 2637 | 1299 | 154 | 0 |
| B3 | 3970 | 3673 | 3758 | 3461 | 4196 | 3484 | 3652 | 2574 | 276 | 2 |
| B2 | 4127 | 3975 | 3786 | 3621 | 3415 | 3068 | 3797 | 2654 | 650 | 1 |
| B1 | 3953 | 4213 | 3135 | 4059 | 4085 | 3554 | 3619 | 2956 | 755 | 4 |
| B0 | 4122 | 5402 | 5149 | 5598 | 4723 | 4546 | 4296 | 3475 | 1078 | 1 |

TABLE 40
XX-IV-182 (7 d)

| | A0 | A1 | A2 | A3 | A4 | A5 | A6 | A7 | A8 | A9 |
|---|---|---|---|---|---|---|---|---|---|---|
| B9 | 194 | 88 | 191 | 136 | 175 | 201 | 203 | 52 | 18 | 3 |
| B8 | 716 | 655 | 709 | 543 | 534 | 447 | 316 | 192 | 10 | 2 |
| B7 | 1068 | 983 | 962 | 844 | 938 | 745 | 605 | 220 | 14 | 3 |
| B6 | 1741 | 1378 | 1362 | 1249 | 1284 | 972 | 977 | 379 | 25 | 8 |
| B5 | 2109 | 1839 | 1815 | 1749 | 1644 | 1620 | 1326 | 806 | 24 | 3 |
| B4 | 3097 | 2977 | 2947 | 3070 | 3010 | 2719 | 2267 | 1220 | 399 | 11 |
| B3 | 2995 | 4635 | 4202 | 4028 | 3463 | 3656 | 2838 | 1757 | 482 | 5 |
| B2 | 3863 | 3654 | 4246 | 4004 | 3429 | 3076 | 3078 | 1973 | 759 | 14 |
| B1 | 3806 | 4090 | 3935 | 3993 | 3759 | 3804 | 3047 | 1835 | 798 | 3 |
| B0 | 2952 | 4034 | 4692 | 4103 | 4991 | 4074 | 2926 | 2085 | 1052 | 7 |

TABLE 41
m1600 (7 d)

| | A0 | A1 | A2 | A3 | A4 | A5 | A6 | A7 | A8 | A9 |
|---|---|---|---|---|---|---|---|---|---|---|
| B9 | 143 | 112 | 120 | 73 | 89 | 76 | 76 | 119 | 43 | 12 |
| B8 | 693 | 457 | 398 | 370 | 394 | 307 | 328 | 234 | 226 | 12 |
| B7 | 812 | 643 | 649 | 564 | 614 | 580 | 374 | 255 | 342 | 14 |
| B6 | 1500 | 967 | 990 | 736 | 928 | 667 | 589 | 433 | 315 | 16 |
| B5 | 2919 | 2113 | 2163 | 2060 | 1571 | 1477 | 968 | 550 | 98 | 13 |
| B4 | 5606 | 4696 | 3726 | 3800 | 2287 | 2862 | 1955 | 1141 | 200 | 12 |
| B3 | 2457 | 5339 | 4467 | 4524 | 3406 | 2993 | 2907 | 1302 | 347 | 11 |
| B2 | 4478 | 4668 | 4263 | 4253 | 3898 | 3420 | 2831 | 1591 | 314 | 11 |
| B1 | 2362 | 4454 | 4457 | 3993 | 3889 | 3143 | 2348 | 1502 | 369 | 19 |
| B0 | 471 | 1893 | 2130 | 3491 | 5488 | 5243 | 4346 | 2600 | 315 | 14 |

TABLE 42
XX-IV-153 (3 d)

| | A0 | A1 | A2 | A3 | A4 | A5 | A6 | A7 | A8 | A9 |
|---|---|---|---|---|---|---|---|---|---|---|
| B9 | 265 | 234 | 255 | 252 | 301 | 249 | 162 | 34 | 20 | 12 |
| B8 | 625 | 479 | 526 | 528 | 425 | 475 | 406 | 235 | 96 | 7 |
| B7 | 821 | 828 | 716 | 711 | 771 | 817 | 614 | 355 | 147 | 10 |
| B6 | 893 | 938 | 841 | 878 | 885 | 649 | 699 | 505 | 214 | 12 |
| B5 | 1454 | 1325 | 1153 | 1317 | 1327 | 1471 | 1191 | 944 | 244 | 11 |
| B4 | 1963 | 1900 | 1823 | 1695 | 1732 | 1847 | 1809 | 1544 | 332 | 3 |
| B3 | 2073 | 1772 | 1998 | 1991 | 2153 | 1338 | 1849 | 1413 | 317 | 4 |
| B2 | 1018 | 1866 | 1864 | 2081 | 1954 | 1729 | 1602 | 1226 | 389 | 15 |
| B1 | 1442 | 702 | 1349 | 1429 | 1554 | 1628 | 1642 | 1201 | 302 | 4 |
| B0 | 2449 | 2404 | 3545 | 3359 | 2839 | 3303 | 2330 | 2729 | 1186 | 18 |

TABLE 43
X-SK-8266 (3 d)

| | A0 | A1 | A2 | A3 | A4 | A5 | A6 | A7 | A8 | A9 |
|---|---|---|---|---|---|---|---|---|---|---|
| B9 | 260 | 213 | 183 | 223 | 217 | 191 | 96 | 64 | 47 | 3 |
| B8 | 322 | 283 | 217 | 218 | 241 | 231 | 230 | 107 | 34 | 11 |
| B7 | 325 | 284 | 264 | 257 | 314 | 327 | 236 | 130 | 61 | 21 |
| B6 | 432 | 416 | 287 | 402 | 436 | 435 | 358 | 217 | 104 | 22 |
| B5 | 707 | 654 | 610 | 675 | 664 | 625 | 566 | 324 | 74 | 30 |
| B4 | 1171 | 810 | 891 | 502 | 785 | 731 | 784 | 508 | 166 | 18 |
| B3 | 1598 | 1072 | 827 | 741 | 1007 | 789 | 739 | 488 | 90 | 30 |
| B2 | 1534 | 952 | 912 | 875 | 912 | 747 | 728 | 625 | 117 | 57 |
| B1 | 1476 | 947 | 713 | 780 | 718 | 818 | 848 | 663 | 152 | 19 |
| B0 | 1717 | 985 | 782 | 766 | 866 | 856 | 619 | 558 | 161 | 25 |

TABLE 44
X-SK-8266 (7 d)

| | A0 | A1 | A2 | A3 | A4 | A5 | A6 | A7 | A8 | A9 |
|---|---|---|---|---|---|---|---|---|---|---|
| B9 | 72 | 99 | 61 | 67 | 24 | 24 | 30 | 20 | 25 | 8 |
| B8 | 110 | 117 | 90 | 114 | 61 | 95 | 68 | 35 | 35 | 16 |
| B7 | 99 | 119 | 110 | 96 | 121 | 94 | 55 | 55 | 42 | 14 |
| B6 | 224 | 115 | 185 | 167 | 176 | 113 | 58 | 105 | 8 | 34 |
| B5 | 514 | 319 | 379 | 314 | 369 | 307 | 324 | 136 | 60 | 12 |
| B4 | 2316 | 1026 | 952 | 621 | 617 | 846 | 944 | 320 | 20 | 13 |
| B3 | 2548 | 1452 | 1133 | 1027 | 930 | 863 | 798 | 279 | 37 | 27 |
| B2 | 3359 | 1655 | 1236 | 989 | 992 | 821 | 632 | 211 | 58 | 38 |
| B1 | 2523 | 1255 | 1121 | 1083 | 852 | 815 | 604 | 396 | 118 | 16 |
| B0 | 1679 | 1643 | 1100 | 1193 | 998 | 706 | 868 | 371 | 99 | 40 |

TABLE 45
m1730 (7 d)

| | A0 | A1 | A2 | A3 | A4 | A5 | A6 | A7 | A8 | A9 |
|---|---|---|---|---|---|---|---|---|---|---|
| B9 | 5 | 5 | 5 | 8 | 8 | 6 | 5 | 22 | 27 | 16 |
| B8 | 3 | 4 | 4 | 5 | 3 | 2 | 8 | 15 | 42 | 8 |
| B7 | 5 | 4 | 2 | 0 | 2 | 8 | 8 | 13 | 36 | 17 |
| B6 | 3 | 7 | 4 | 6 | 6 | 6 | 9 | 9 | 45 | 17 |
| B5 | 52 | 82 | 63 | 43 | 41 | 24 | 14 | 15 | 44 | 10 |
| B4 | 599 | 1720 | 1785 | 1388 | 1463 | 773 | 174 | 13 | 43 | 7 |
| B3 | 2390 | 2886 | 3265 | 3212 | 2483 | 2057 | 894 | 37 | 28 | 9 |
| B2 | 2463 | 3560 | 3295 | 2959 | 2768 | 2029 | 749 | 40 | 25 | 12 |
| B1 | 3052 | 3399 | 3610 | 3583 | 3250 | 2337 | 1141 | 48 | 29 | 6 |
| B0 | 3463 | 4260 | 5214 | 5497 | 5598 | 4127 | 2393 | 258 | 19 | 5 |

TABLE 46
X-R-IV-109 (7 d)

| | A0 | A1 | A2 | A3 | A4 | A5 | A6 | A7 | A8 | A9 |
|---|---|---|---|---|---|---|---|---|---|---|
| B9 | 37 | 25 | 19 | 32 | 33 | 22 | 36 | 27 | 16 | 33 |
| B8 | 188 | 175 | 175 | 178 | 209 | 233 | 300 | 392 | 118 | 28 |
| B7 | 430 | 341 | 390 | 287 | 271 | 472 | 546 | 642 | 164 | 29 |
| B6 | 483 | 287 | 439 | 502 | 399 | 766 | 727 | 1143 | 333 | 135 |
| B5 | 949 | 774 | 801 | 652 | 258 | 1052 | 1288 | 1490 | 598 | 56 |
| B4 | 3695 | 2466 | 2177 | 2065 | 932 | 2208 | 2880 | 2431 | 795 | 98 |
| B3 | 2605 | 3405 | 3511 | 4065 | 3407 | 3186 | 4361 | 3562 | 1199 | 57 |
| B2 | 3096 | 4304 | 3628 | 3196 | 3584 | 3770 | 4295 | 3860 | 1410 | 22 |
| B1 | 3139 | 3299 | 2951 | 2903 | 4018 | 4071 | 4909 | 3925 | 1425 | 174 |
| B0 | 5287 | 7093 | 3309 | 3369 | 8255 | 7783 | 7909 | 5856 | 2103 | 61 |

TABLE 47
XX-IV-153 (7 d)

| | A0 | A1 | A2 | A3 | A4 | A5 | A6 | A7 | A8 | A9 |
|---|---|---|---|---|---|---|---|---|---|---|
| B9 | 28 | 49 | 41 | 38 | 39 | 47 | 53 | 48 | 20 | 13 |
| B8 | 28 | 48 | 48 | 45 | 50 | 53 | 59 | 50 | 40 | 4 |
| B7 | 90 | 72 | 40 | 61 | 58 | 77 | 66 | 66 | 59 | 8 |
| B6 | 114 | 61 | 104 | 92 | 95 | 148 | 86 | 80 | 51 | 13 |
| B5 | 138 | 119 | 97 | 203 | 147 | 245 | 173 | 136 | 98 | 17 |
| B4 | 8572 | 8348 | 3056 | 7879 | 7765 | 8096 | 8512 | 5342 | 529 | 11 |
| B3 | 9108 | 8837 | 8710 | 9755 | 8690 | 2713 | 7598 | 5710 | 225 | 9 |
| B2 | 7675 | 8636 | 8808 | 8558 | 7832 | 9902 | 8177 | 3042 | 847 | 11 |
| B1 | 8286 | 8813 | 8992 | 9256 | 8050 | 8196 | 8044 | 5960 | 454 | 19 |
| B0 | 7758 | 6964 | 8639 | 9007 | 8949 | 8707 | 6201 | 5199 | 1095 | 13 |

TABLE 48
X-SK-5486-R (7 d)

| | A0 | A1 | A2 | A3 | A4 | A5 | A6 | A7 | A8 | A9 |
|---|---|---|---|---|---|---|---|---|---|---|
| B9 | 53 | 61 | 48 | 47 | 39 | 49 | 42 | 22 | 11 | 10 |
| B8 | 105 | 102 | 128 | 132 | 153 | 146 | 80 | 88 | 54 | 6 |
| B7 | 115 | 176 | 188 | 191 | 202 | 187 | 93 | 90 | 34 | 22 |
| B6 | 215 | 226 | 247 | 203 | 302 | 214 | 271 | 201 | 52 | 33 |
| B5 | 450 | 469 | 537 | 592 | 663 | 518 | 398 | 245 | 60 | 40 |
| B4 | 3914 | 4752 | 4394 | 4146 | 4090 | 3409 | 3295 | 1313 | 81 | 30 |
| B3 | 9069 | 8915 | 9771 | 9515 | 8986 | 8612 | 7187 | 4863 | 152 | 37 |
| B2 | 9257 | 10025 | 10404 | 10038 | 10548 | 8922 | 8595 | 4929 | 337 | 48 |
| B1 | 9754 | 9906 | 10564 | 10158 | 9855 | 9274 | 8138 | 4818 | 324 | 36 |
| B0 | 3970 | 9051 | 5484 | 10118 | 9117 | 9577 | 8739 | 3575 | 1008 | 23 |

TABLE 49
X-SK-6731 (7 d)

| | A0 | A1 | A2 | A3 | A4 | A5 | A6 | A7 | A8 | A9 |
|---|---|---|---|---|---|---|---|---|---|---|
| B9 | 1 | 3 | 1 | 1 | 4 | 6 | 4 | 0 | 2 | 1 |
| B8 | 42 | 33 | 28 | 20 | 20 | 14 | 20 | 2 | 1 | 1 |
| B7 | 31 | 40 | 27 | 38 | 24 | 34 | 23 | 9 | 3 | 0 |
| B6 | 66 | 39 | 51 | 55 | 51 | 68 | 33 | 23 | 4 | 0 |
| B5 | 604 | 354 | 377 | 240 | 230 | 217 | 153 | 50 | 11 | 0 |
| B4 | 4670 | 2566 | 2609 | 1976 | 1659 | 1435 | 953 | 172 | 19 | 0 |
| B3 | 8277 | 4954 | 3685 | 3355 | 2652 | 2600 | 1774 | 391 | 6 | 0 |
| B2 | 7321 | 5165 | 4287 | 3751 | 2679 | 2525 | 1628 | 488 | 5 | 0 |
| B1 | 7019 | 4807 | 4075 | 3273 | 3031 | 2598 | 1564 | 513 | 2 | 0 |
| B0 | 9279 | 6018 | 4739 | 3509 | 3668 | 2826 | 1860 | 834 | 11 | 2 |

TABLE 50
m1371 (3 d)

| | A0 | A1 | A2 | A3 | A4 | A5 | A6 | A7 | A8 | A9 |
|---|---|---|---|---|---|---|---|---|---|---|
| B9 | 113 | 138 | 97 | 121 | 107 | 109 | 85 | 36 | 27 | 31 |
| B8 | 273 | 308 | 242 | 319 | 285 | 237 | 244 | 252 | 35 | 0 |
| B7 | 322 | 324 | 273 | 326 | 313 | 351 | 325 | 207 | 12 | 0 |
| B6 | 346 | 449 | 427 | 373 | 411 | 422 | 438 | 203 | 39 | 2 |
| B5 | 576 | 543 | 552 | 571 | 537 | 632 | 522 | 377 | 42 | 12 |
| B4 | 623 | 762 | 656 | 784 | 642 | 710 | 956 | 705 | 54 | 4 |
| B3 | 629 | 635 | 686 | 663 | 775 | 753 | 1047 | 728 | 55 | 23 |
| B2 | 713 | 694 | 703 | 730 | 784 | 728 | 893 | 549 | 42 | 25 |
| B1 | 512 | 754 | 583 | 808 | 880 | 718 | 783 | 517 | 54 | 13 |
| B0 | 700 | 771 | 740 | 857 | 805 | 1006 | 836 | 688 | 61 | 4 |

TABLE 51
m1730 (3 d)

| | A0 | A1 | A2 | A3 | A4 | A5 | A6 | A7 | A8 | A9 |
|---|---|---|---|---|---|---|---|---|---|---|
| B9 | 11 | 10 | 4 | 2 | 8 | 5 | 1 | 31 | 14 | 0 |
| B8 | 39 | 32 | 26 | 12 | 11 | 20 | 6 | 11 | 10 | 5 |
| B7 | 51 | 44 | 26 | 42 | 30 | 15 | 14 | 14 | 22 | 9 |
| B6 | 151 | 127 | 96 | 88 | 59 | 64 | 24 | 7 | 31 | 2 |
| B5 | 669 | 750 | 840 | 689 | 760 | 525 | 179 | 28 | 23 | 14 |
| B4 | 2329 | 2925 | 2886 | 2149 | 2409 | 2286 | 1429 | 149 | 9 | 12 |
| B3 | 2830 | 3105 | 3486 | 3022 | 3175 | 2685 | 1918 | 310 | 38 | 8 |
| B2 | 2821 | 3144 | 3435 | 3228 | 3224 | 2746 | 2111 | 415 | 43 | 15 |
| B1 | 3513 | 3268 | 3661 | 3401 | 3242 | 2971 | 2505 | 322 | 22 | 6 |
| B0 | 4928 | 4546 | 3818 | 4179 | 3944 | 4072 | 3653 | 1224 | 19 | 23 |

TABLE 52
m877 (7 d)

| | A0 | A1 | A2 | A3 | A4 | A5 | A6 | A7 | A8 | A9 |
|---|---|---|---|---|---|---|---|---|---|---|
| B9 | 185 | 20 | 22 | 5 | 6 | 12 | 5 | 2 | 0 | 1 |
| B8 | 814 | 223 | 236 | 105 | 83 | 69 | 27 | 19 | 9 | 1 |
| B7 | 1388 | 462 | 383 | 290 | 199 | 139 | 63 | 24 | 19 | 1 |
| B6 | 1908 | 961 | 680 | 583 | 381 | 281 | 137 | 19 | 24 | 0 |
| B5 | 4383 | 1789 | 1371 | 1265 | 861 | 843 | 295 | 48 | 32 | 1 |
| B4 | 5862 | 3183 | 2517 | 2529 | 1806 | 1327 | 541 | 39 | 13 | 1 |
| B3 | 7279 | 3759 | 3270 | 3124 | 2663 | 2067 | 1468 | 332 | 9 | 1 |
| B2 | 5532 | 4103 | 3815 | 2733 | 2960 | 2362 | 1860 | 556 | 31 | 0 |
| B1 | 4746 | 3688 | 3290 | 2657 | 2573 | 2177 | 1639 | 781 | 20 | 2 |
| B0 | 6011 | 3350 | 3024 | 2636 | 2367 | 2008 | 1592 | 917 | 32 | 0 |

TABLE 53
m3320 (7 d)

| | A0 | A1 | A2 | A3 | A4 | A5 | A6 | A7 | A8 | A9 |
|---|---|---|---|---|---|---|---|---|---|---|
| B9 | 1 | 12 | 7 | 10 | 44 | 4 | 11 | 10 | 3 | 7 |
| B8 | 29 | 30 | 12 | 17 | 20 | 37 | 22 | 18 | 9 | 1 |
| B7 | 26 | 27 | 22 | 30 | 31 | 28 | 37 | 19 | 9 | 3 |
| B6 | 38 | 40 | 26 | 44 | 52 | 42 | 33 | 21 | 10 | 7 |
| B5 | 235 | 200 | 203 | 126 | 212 | 197 | 162 | 32 | 10 | 5 |
| B4 | 4084 | 2863 | 2869 | 2824 | 2519 | 2939 | 2645 | 1498 | 17 | 0 |
| B3 | 16626 | 10918 | 10124 | 8541 | 9882 | 8078 | 8799 | 4974 | 30 | 4 |
| B2 | 16930 | 12456 | 12090 | 9527 | 10090 | 9997 | 11230 | 4831 | 49 | 4 |
| B1 | 17469 | 11471 | 11449 | 9934 | 10910 | 11491 | 11146 | 6334 | 53 | 3 |
| B0 | 22081 | 14399 | 13135 | 15293 | 8420 | 5988 | 940 | 450 | 1820 | 1 |

TABLE 54
X-R-IV-109 (3 d)

| | A0 | A1 | A2 | A3 | A4 | A5 | A6 | A7 | A8 | A9 |
|---|---|---|---|---|---|---|---|---|---|---|
| B9 | 1471 | 1466 | 1501 | 1602 | 1676 | 1794 | 1398 | 1515 | 68 | 16 |
| B8 | 1659 | 1706 | 1839 | 1630 | 1825 | 1954 | 1968 | 1901 | 812 | 6 |
| B7 | 1533 | 2270 | 2237 | 2166 | 2224 | 2087 | 2273 | 2284 | 714 | 7 |
| B6 | 2758 | 2518 | 2926 | 2685 | 2544 | 2379 | 2678 | 2985 | 1126 | 6 |
| B5 | 2626 | 3687 | 3533 | 3200 | 3527 | 3723 | 3654 | 2287 | 1870 | 1 |
| B4 | 4139 | 4184 | 4454 | 4353 | 4755 | 5017 | 4927 | 4752 | 2255 | 6 |
| B3 | 4480 | 4491 | 4588 | 4587 | 4602 | 5014 | 4953 | 4743 | 2461 | 3 |
| B2 | 4024 | 4404 | 4609 | 4414 | 4709 | 4840 | 4834 | 4639 | 2587 | 3 |
| B1 | 4195 | 4612 | 4542 | 4638 | 4777 | 5116 | 4860 | 4664 | 2590 | 6 |
| B0 | 4941 | 5128 | 5836 | 5521 | 4885 | 6309 | 6138 | 5751 | 2981 | 7 |

TABLE 55
m1339 (3 d)

| | A0 | A1 | A2 | A3 | A4 | A5 | A6 | A7 | A8 | A9 |
|---|---|---|---|---|---|---|---|---|---|---|
| B9 | 1223 | 0 | 1388 | 1583 | 1364 | 1363 | 788 | 0 | 3 | 1 |
| B8 | 1626 | 1144 | 1102 | 947 | 1188 | 1036 | 882 | 136 | 3 | 1 |
| B7 | 1335 | 1431 | 995 | 1154 | 1443 | 1523 | 877 | 285 | 8 | 1 |
| B6 | 2353 | 2049 | 1565 | 1515 | 2108 | 1436 | 1066 | 387 | 8 | 0 |
| B5 | 2809 | 2337 | 2513 | 3019 | 2221 | 2553 | 1983 | 731 | 17 | 0 |
| B4 | 3264 | 2809 | 3051 | 3208 | 3243 | 3182 | 3254 | 1522 | 60 | 2 |
| B3 | 3585 | 3150 | 3079 | 3499 | 3127 | 2871 | 3718 | 2054 | 110 | 1 |
| B2 | 2857 | 2713 | 3164 | 2818 | 3359 | 2725 | 4098 | 2745 | 272 | 1 |
| B1 | 3028 | 3038 | 3297 | 3158 | 3380 | 3308 | 3549 | 2517 | 189 | 0 |
| B0 | 4770 | 3945 | 4050 | 3763 | 4321 | 4701 | 4063 | 3346 | 731 | 0 |

TABLE 56
m3285 (7 d)

| | A0 | A1 | A2 | A3 | A4 | A5 | A6 | A7 | A8 | A9 |
|---|---|---|---|---|---|---|---|---|---|---|
| B9 | 103 | 166 | 223 | 359 | 231 | 425 | 452 | 585 | 381 | 665 |
| B8 | 149 | 142 | 203 | 228 | 349 | 638 | 873 | 643 | 646 | 952 |
| B7 | 96 | 120 | 274 | 316 | 428 | 601 | 828 | 591 | 812 | 1173 |
| B6 | 64 | 151 | 242 | 217 | 314 | 784 | 854 | 542 | 640 | 988 |
| B5 | 81 | 126 | 196 | 236 | 410 | 801 | 849 | 685 | 650 | 651 |
| B4 | 104 | 102 | 183 | 181 | 285 | 789 | 1003 | 773 | 942 | 1230 |
| B3 | 58 | 64 | 90 | 94 | 227 | 577 | 617 | 593 | 829 | 1162 |
| B2 | 63 | 103 | 57 | 70 | 133 | 190 | 329 | 455 | 265 | 996 |
| B1 | 18 | 13 | 54 | 53 | 62 | 84 | 125 | 162 | 297 | 916 |
| B0 | 15 | 5 | 4 | 12 | 10 | 10 | 24 | 15 | 14 | 11 |

TABLE 57
m1675 (3d)

| | A0 | A1 | A2 | A3 | A4 | A5 | A6 | A7 | A8 | A9 |
|---|---|---|---|---|---|---|---|---|---|---|
| B9 | 44 | 26 | 38 | 34 | 54 | 32 | 35 | 7 | 27 | 5 |
| B8 | 94 | 102 | 101 | 81 | 152 | 136 | 144 | 72 | 11 | 3 |
| B7 | 103 | 131 | 118 | 138 | 192 | 184 | 227 | 137 | 3 | 9 |
| B6 | 93 | 105 | 109 | 101 | 154 | 165 | 199 | 216 | 3 | 4 |
| B5 | 131 | 126 | 143 | 163 | 220 | 190 | 248 | 181 | 7 | 3 |
| B4 | 149 | 155 | 214 | 145 | 246 | 274 | 314 | 204 | 7 | 7 |
| B3 | 138 | 201 | 196 | 194 | 260 | 263 | 325 | 289 | 9 | 12 |
| B2 | 181 | 173 | 211 | 317 | 266 | 267 | 310 | 254 | 23 | 5 |
| B1 | 139 | 179 | 182 | 158 | 333 | 199 | 306 | 218 | 9 | 5 |
| B0 | 504 | 612 | 661 | 766 | 762 | 420 | 817 | 846 | 73 | 9 |

TABLE 58
XX-IV-113 (3 d)

| | A0 | A1 | A2 | A3 | A4 | A5 | A6 | A7 | A8 | A9 |
|---|---|---|---|---|---|---|---|---|---|---|
| B9 | 600 | 637 | 800 | 832 | 660 | 346 | 300 | 105 | 16 | 7 |
| B8 | 1637 | 1643 | 1541 | 1555 | 1680 | 1250 | 953 | 665 | 148 | 9 |
| B7 | 1514 | 1903 | 1845 | 1896 | 1923 | 1650 | 1340 | 735 | 265 | 8 |
| B6 | 2012 | 1955 | 1897 | 1729 | 1955 | 1529 | 1413 | 983 | 414 | 8 |
| B5 | 1730 | 2067 | 2187 | 1973 | 2225 | 2040 | 1761 | 1273 | 447 | 4 |
| B4 | 1920 | 1610 | 2582 | 1751 | 2412 | 2031 | 2749 | 1436 | 485 | 11 |
| B3 | 1909 | 2167 | 2780 | 2668 | 2333 | 2723 | 2551 | 1777 | 553 | 13 |
| B2 | 2026 | 2392 | 2425 | 2516 | 2652 | 2387 | 1611 | 1725 | 563 | 6 |
| B1 | 2186 | 1655 | 1881 | 1876 | 2421 | 2759 | 2425 | 1699 | 605 | 2 |
| B0 | 4890 | 4668 | 4570 | 3075 | 5106 | 5496 | 6086 | 5260 | 2907 | 9 |

TABLE 59
m1717 (7d)

| | A0 | A1 | A2 | A3 | A4 | A5 | A6 | A7 | A8 | A9 |
|---|---|---|---|---|---|---|---|---|---|---|
| B9 | 53 | 60 | 48 | 49 | 32 | 28 | 7 | 3 | 5 | 1 |
| B8 | 361 | 444 | 472 | 405 | 361 | 364 | 133 | 9 | 17 | 0 |
| B7 | 538 | 585 | 476 | 723 | 628 | 397 | 291 | 43 | 25 | 4 |
| B6 | 1033 | 995 | 942 | 930 | 919 | 679 | 428 | 107 | 9 | 1 |
| B5 | 1810 | 1397 | 1864 | 1309 | 1554 | 1321 | 737 | 69 | 4 | 2 |
| B4 | 2377 | 6494 | 5263 | 4346 | 4291 | 4008 | 2808 | 439 | 12 | 4 |
| B3 | 2172 | 6475 | 6506 | 6141 | 5928 | 4514 | 2145 | 1367 | 13 | 1 |
| B2 | 2149 | 6013 | 7131 | 6203 | 5290 | 5987 | 4742 | 2024 | 25 | 1 |
| B1 | 2521 | 6432 | 6411 | 6085 | 6481 | 6272 | 4572 | 2069 | 26 | 1 |
| B0 | 1166 | 3890 | 5665 | 5980 | 6794 | 5897 | 4734 | 2420 | 147 | 0 |

TABLE 60
X-SK-5486-R (3 d)

| | A0 | A1 | A2 | A3 | A4 | A5 | A6 | A7 | A8 | A9 |
|---|---|---|---|---|---|---|---|---|---|---|
| B9 | 807 | 880 | 856 | 753 | 803 | 797 | 571 | 254 | 41 | 13 |
| B8 | 1403 | 1402 | 1660 | 1523 | 1468 | 1518 | 1328 | 954 | 223 | 41 |
| B7 | 1440 | 1413 | 1485 | 1543 | 1446 | 1414 | 1483 | 1099 | 299 | 74 |
| B6 | 1590 | 1687 | 1788 | 1712 | 1649 | 1606 | 1611 | 1309 | 211 | 35 |
| B5 | 2541 | 2551 | 2643 | 2940 | 2641 | 2574 | 2591 | 1987 | 378 | 89 |
| B4 | 3959 | 3996 | 3884 | 3497 | 3438 | 3869 | 4035 | 2935 | 488 | 34 |
| B3 | 4572 | 4362 | 4432 | 4451 | 4378 | 4489 | 4434 | 3429 | 939 | 57 |
| B2 | 4412 | 4798 | 4155 | 5023 | 4747 | 4682 | 5086 | 3938 | 1365 | 86 |
| B1 | 4810 | 4630 | 4520 | 4525 | 4447 | 3983 | 4676 | 3699 | 980 | 53 |
| B0 | 5290 | 5690 | 5181 | 6450 | 5880 | 5687 | 5636 | 5695 | 2554 | 34 |

Conclusion: A strong synergy between ASNase and ibrutinib was found in 72% of these PDXs at concentrations between about 0.01 μM and about 10 μM ibrutinib.

### Example 5—Prednisone and Ibrutinib Effect on Nalm6, Sem and 697 Cells

A series of experiments was conducted to test for the specificity of the synergistic interaction between BTK inhibitor ibrutinib and asparaginase. Synthetic glucocorticoids (GCs, e.g. prednisone, dexamethasone) are essential drugs for the treatment of ALL. To test whether BTK inhibition could modulate the response to GCs, the effect of combination treatment was tested. First, Nalm6 cells were exposed to 1 μl of prednisone in the absence or presence of Ibrutinib for 7 days. Next, cell viability was assayed by testing for membrane integrity using a amine-dye followed by flowcytometry. This showed that although combining prednisone treatment with the highest teste dose of 10 μM Ibrutinib resulted in enhanced cell death, this is at best a modest effect when compared to the synergy between asparaginase and Ibrutinib. Similarly, in alternative assays, measuring cell death by testing for PARP cleavage using western blot or DNA fragmentation using Hoechst staining followed by flow cytometry, only a small effect of combination treatment was observed. See FIGS. 6A to 6C.

### Example 6—ASNase and ibrutinib Combination Therapy to Treat Human Leukemia in an Immune Compromised Mouse Model

Objective. To study the safety and efficacy of the ASNase+ibrutinib combination treatment. Compounds: ibrutinib (Sellekchem), 25 mg/kg, dissolved in 5% DMSO+30% PEG 300+5% Tween 80+ddH2O; PEG-Asparaginase (ONCASPAR®), 300 IU/kg in phosphate buffered saline solution. Power calculation: Using a quite modest expected minimal effect size of 20% (a relevant effect in median survival time between the groups), a standard deviation <10%, an alpha-value of 0.05 and a power of 80%, this results in 7 mice per group.

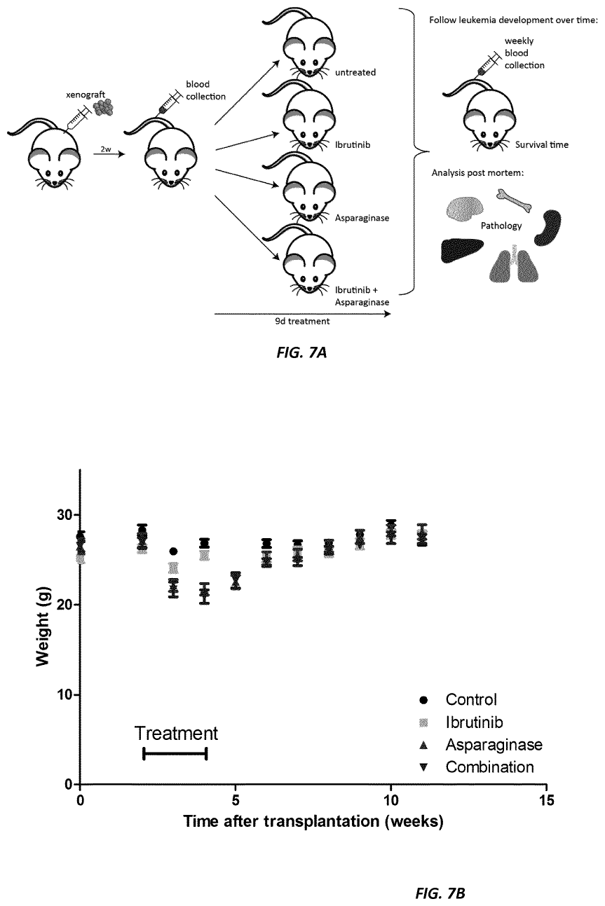

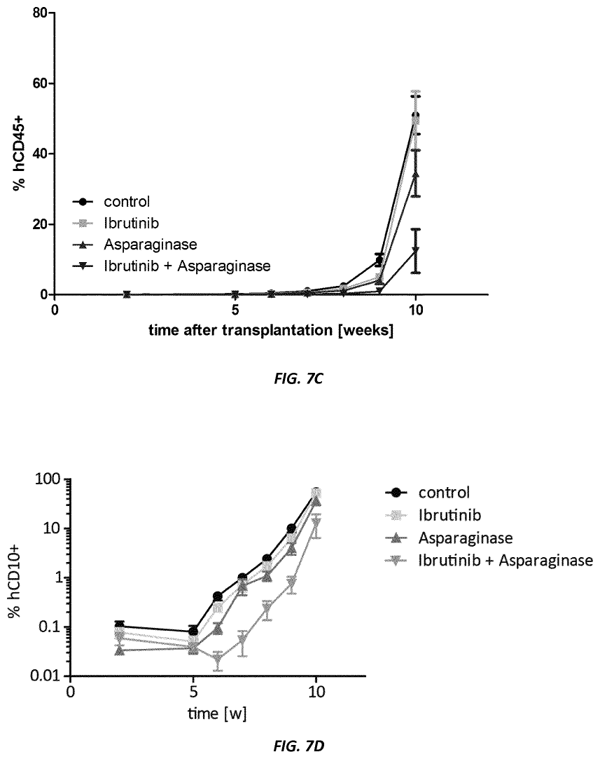

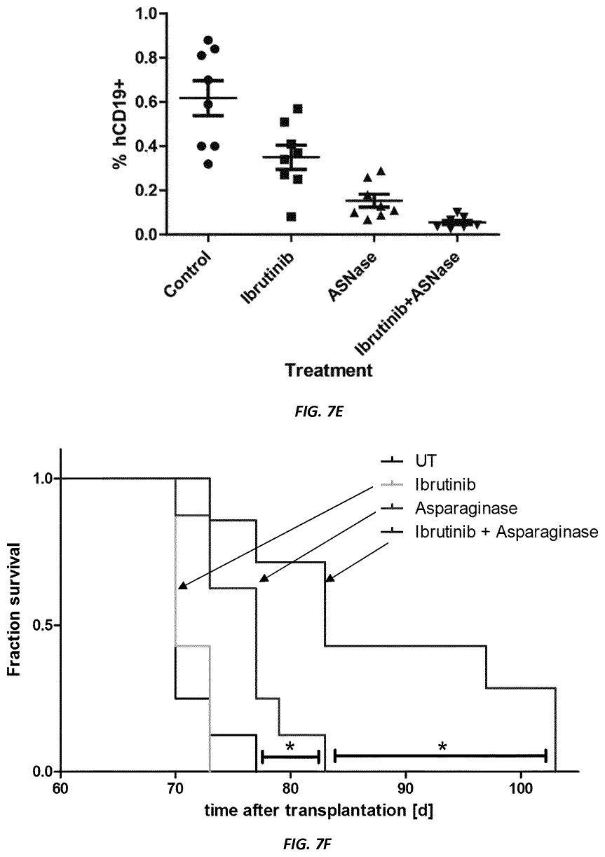

Procedure. The experimental design is schematized in FIG. 7A. Briefly, thirty-two (32) NOD.Cg-Prkdcscid I2rgtm1Wjl/SzJ (“NSG”) mice were injected intrafemorally with 500,000 leukemic blasts (2 injections/mouse) (Human Acute Lymphoblastic Leukemia). Two weeks after transplantation, mice received treatment for 9 consecutive days: 1) daily (9 days) oral administration of ibrutinib (groups 2 and 4) or vehicle (groups 1 and 3) via gavage; 2) IV administration of ONCASPAR® (groups 3 and 4) or vehicle (groups 1 and 2) on days 1, 4 and 7. During and after treatment, mice were weighed weekly, and their welfare was evaluated daily. Blood samples were taken weekly for assessment of the leukemia burden. Termination was predefined with a leukemia burden reaching 50% leukemic blasts in peripheral blood as measured by flow cytometry or euthanasia because of overt signs of discomfort due to disease.

The primary outcome of this experiment was event-free survival, where event was defined as early termination because of other causes will lead to censoring or exclusion from the experiment. Differences in event-free survival were analyzed using a Kaplan-Meier plot and significance was tested using the log-rank test.

Results. Thirty-one (31) of the thirty-two (32) mice were successfully engrafted with human leukemia. None of the mice showed treatment related signs of discomfort other than weight loss. In line with previous experiments, mice treated with ASNase experienced a strong but transient weight loss (FIG. 7B). ibrutinib treatment resulted in a mild and transient drop in bodyweight. Combination treated mice did not show any signs of additional discomfort in comparison to the PEG-Asparaginase treatment arm.

Weekly analysis of blood by flow cytometry showed that 5 weeks after transplantation, human leukemic blasts markers were detectable in control treated mice as identified by expression of human CD45 (FIG. 7C), CD10 (FIG. 7D) and CD19 (FIG. 7E). These numbers increased in time up to >50% which was defined as termination point. For 6 out of 8 control treated mice, this point was reached within 10 weeks after transplantation. Although ibrutinib treatment initially gave a growth retardation, 4/7 mice reached the termination point after 10 weeks and the remaining 3 mice were terminated 1 week later. Asparaginase treatment resulted in a stronger delay in leukemia development, with mice reaching the termination point between 10 and 13 weeks. Combination treatments significantly decreased the % of hCD19+ cells (FIG. 7E), and significantly prolonged survival compared to the ASNase treatment, with mice reaching termination point between 10 and 15 weeks after transplantation (FIG. 7F).

Accordingly, combination treatment of ASNase and ibrutinib significantly delays leukemia development in mice transplanted with human acute lymphoblastic leukemia relative to control mice or mice treated with either compound as a single agent. No overt signs of toxicities as a result of combination treatment were observed, other than the transient weight loss that was also observed in mice treated with ASNase alone.

### Overall Conclusions

The anti-tumor effects of ASNase impinge on changes in cell metabolism that occur as a result of amino acid regulation. Consistent with this notion, we identified genes either directly involved in the amino acid response route (TRIB3) or inhibition of protein translation in response to amino acid starvation (GCN2). Indeed, knockout of GCN2 sensitized cells to ASNase treatment whereas depletion of TRIB3 was sufficient to render these cells more resistant to the effects of ASNase on cell growth.

In addition, we found that Bruton Tyrosine Kinase (BTK), a hematopoietic-cell specific protein kinase acting downstream of the B-cell receptor, protects ALL cells from ASNase-induced apoptosis. Indeed, targeted knockout as well as inhibition by the FDA-approved BTK inhibitor ibrutinib, strongly enhanced ASNase-induced apoptosis in a variety of ALL cell lines (FIG. 1). Moreover, we tested the effect of combination treatment in 35 different patient-derived xenografts samples, mostly representing high risk leukemia cases and covering a wide variety of ALL subtypes. In more than 75% of the cases we observed synergy ranging from moderate to strong with a combination index (CI)<0.8.

In summary, the nutrient stress response pathway is a key modulator of ASNase-induced cell death; BTK provides a strong survival signal under (ASNase-induced) nutrient stress; and the BTK inhibitor ibrutinib potentiated ASNase therapy response in vivo.

### Example 7—AADAS and AADA Combination Therapy to Kill Solid Tumor Cells in an In Vitro Model

Although AADA asparaginase is primarily used in the treatment of leukemia, the responses in various solid tumor types suggest therapeutic efficacy beyond leukemia. Cell line and organoid models of different tumor origin (including, but not limited to pancreatic cancer, colon cancer, ovarium cancer, brain tumors, breast cancer and lymphoma) are tested for sensitivity to AADA therapy (selected from an asparaginase (ASNase), an arginase (ARGase), an arginine deiminase (ADI), a methioninase (METase), an adenosine deaminase (ADA), an IDO, a TDO, fumagillin) in combination with an AADAS (Ibrutinib and other AADAS) using a matrix of concentrations. Cells are seeded in 96 well or 384 well and incubated with the combination of AADA and AADAS at concentrations ranging from 0 to a 10-fold excess of the maximum tolerated dose in vivo. For asparaginase as AADA, these concentrations range from 0 to 100 IU/ml. For Ibrutinib as AADAS, concentrations range from 0 to 100 μM. After incubation for 3-7 days, cell viability is determined using automated microscopy using live cells staining or flowcytometry using 7AAD or amine staining to discriminate between live or dead cells. Quantification of the microscopy images was done using matlab. In a second step the data is analyzed using R-software to calculate a Combination Index (CI). These results demonstrate that AADA and AADAS synergize in killing solid tumor cells at clinically relevant concentrations. Again, for 7 day studies, cells are refed with [particular amino acid]-depleted media at the midway point.

### Example 8—AADAS and AADA Combination Therapy to Kill Solid Tumor Cells in an In Vivo Model

The efficacy of combination therapy of AADA and AADAS depends on the pharmacokinetics and dynamics of the drugs within a tumor type. In vivo models are used to demonstrate synergy at clinically relevant concentrations. Solid tumor models (cell lines and primary patient cells) are either grown in spheroids, organoids or in a growth supporting matrix (matrigel, growth factor containing scaffolds). Next, tumor cells grown as described are transplanted into a host (in most cases an immunocompromised mouse, commonly known as NSG-mice) and allow to grow and to be innervated by the host's blood vessels. When a tumor is established, the host will be treated with control compounds (solvents), AADA, AADAS and a combination thereof in concentrations either used in clinical practice, commonly used in experimental in vivo models or at the maximal tolerable dose using the administration route that is common practice for the compounds. The duration and frequency of administration are adapted to the compound based on pharmacokinetics and dynamics.

In the case where the AADA is PEG-asparaginase, the dose may be about 300 μl/kg, administered IV once every 3 days. In case the AADAS is ibrutinib, the host may be treated with about 25 mg/kg, administered orally once every day. The effect of tumor growth is monitored by in vivo imaging (in case the tumor cells are labelled with a fluorescent or bioluminescent marker), SPECT/CT and in case the tumor is transplanted subcutaneous, by directly measuring tumor size. The effect of control treatment, single treatment with either AADA or AADAS and the combination thereof on tumor development and/or the formation of metastasis is compared using predefined end-points such as tumor size, morbidity or mortality of the host. The results indicate that AADA and AADAS synergize in the treatment of solid tumor cells in in vivo models at clinically relevant concentrations.

### Example 9—Efficacy of AADAS+AADA Against HB-ALL, LOUCY, SupT1 and JURKAT Cells

T-ALL cell lines were tested as described in the above examples, and sensitivity to Asparaginase (SPECTRILA®, the recombinantly produced E. coli ASNase) and Ibrutinib (described above) were tested as detailed in Table 61. HBP-ALL (ThermoFisher); Loucy cells (ATCC® CRL-2629™); and SubT1 (ATCC® CRL-1942′); Jurkat (ATCC® Clone E61).

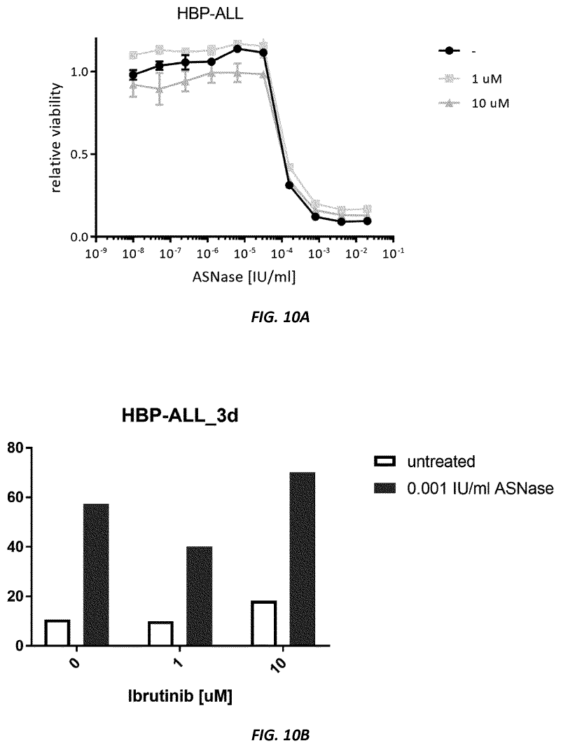

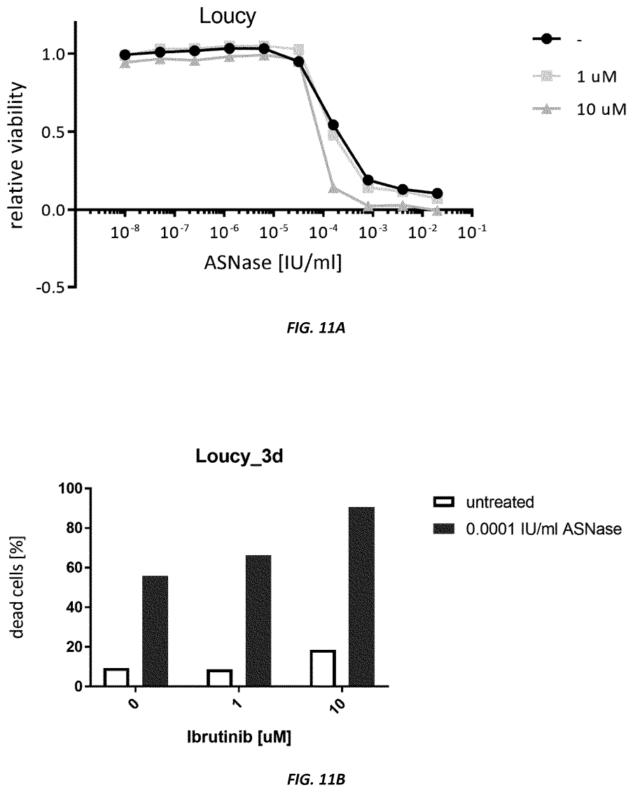

TABLE 61
Treatment details. Cell were incubated as indicated and tested
at either day 3 or 7 (FIGS. 10 to 13 summarize the results)

| | [ASNase] | | [Ibrutinib] | | Assay |
|---|---|---|---|---|---|
| ALL line | tested | | tested | Assay | Day |
| HBP-ALL | Range across | | 0, 1, 10 μM | MTT | |
| | 6.5 logs | | | | |
| HBP-ALL | 0.001 | IU/mL | 0, 1, 10 μM | Cell | 3 |
| | | | | Death | |
| LOUCY | Range across | | 0, 1, 10 μM | MTT | |
| | 6.5 logs | | | | |
| LOUCY | 0.0001 | IU/mL | 0, 1, 10 μM | Cell | 3 |
| | | | | Death | |
| SubT1 | Range across | | 0, 0.1, 1, | MTT | |
| | 8.5 logs | | 10 μM | | |
| SubT1 | 1 | IU/mL | 0, 1, 10 μM | Cell | 3 |
| | | | | Death | |
| SubT1 | 1 | IU/mL | 0, 1, 10 μM | Cell | 7 |
| | | | | Death | |
| JURKAT | Range across | | 0, 0.1, 1, | MTT | |
| | 8.5 logs | | 10 μM | | |
| JURKAT | 1 | IU/mL | 0, 1, 10 μM | Cell | 7 |
| | | | | Death | |

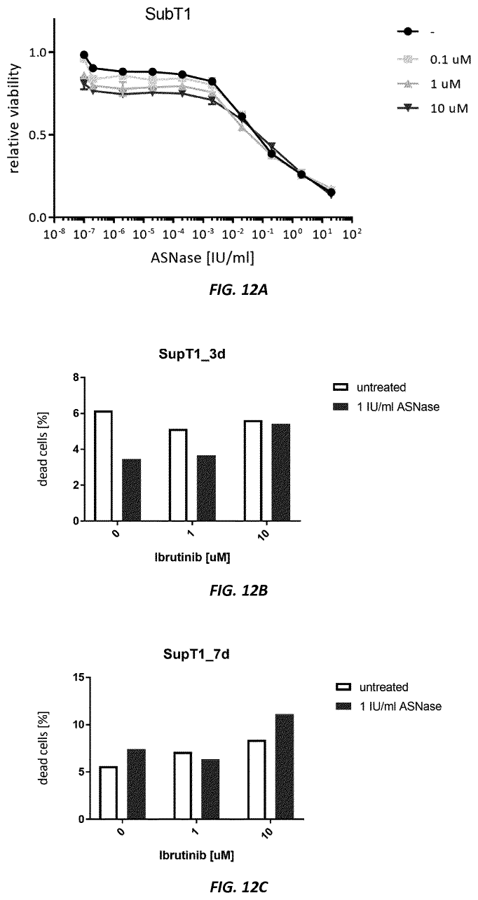

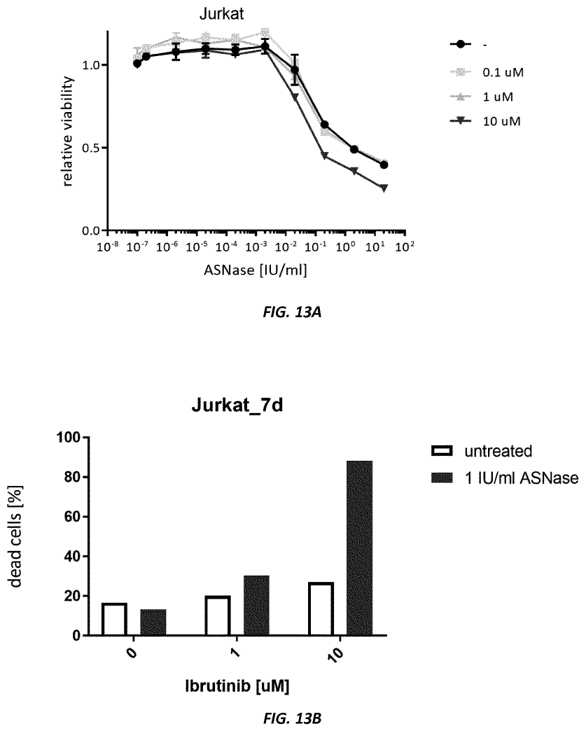

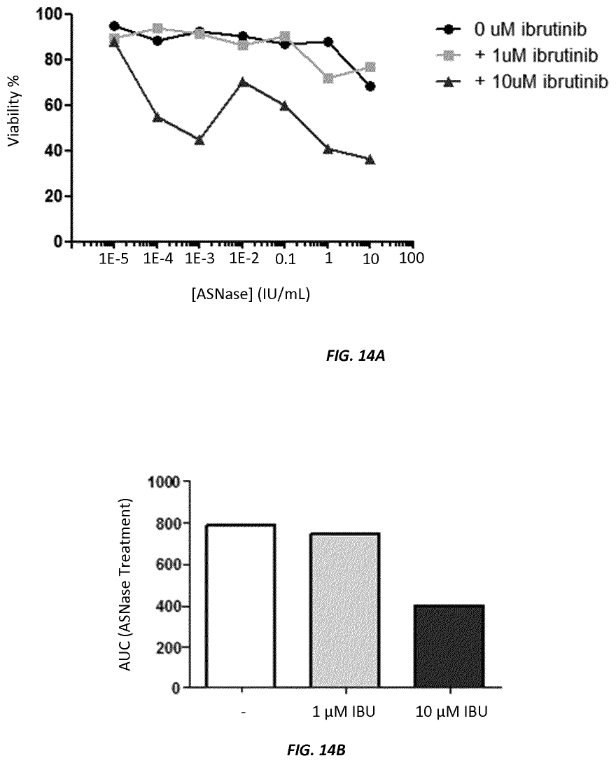

Results. As indicated in FIGS. 10 to 13, cell death (after either 3 or 7 days) was observed as determined by amine exposure. The first two cell lines (HBP-ALL, Loucy) appeared to be extremely sensitive to ASNase as a single agent. Even so, the addition of ibrutinib appeared to produce some synergistic efficacy (FIG. 11A-B). Further, the SupT1 line appeared to be largely unresponsive to any of the treatments (FIGS. 12A-C), while the Jurkat cells exhibited the most prominent “synergistic sensitivity” to the combination (FIGS. 13A-B). And while these results highlight the unpredictable nature of the response/sensitivity of cancer cells to the combined efficacy of AADAS+AADA, the skilled artisan reading this application will now be able to conduct routine tests to evaluate the synergistic—or other supra-additive—efficacy of the combination against any other cell/cell type.

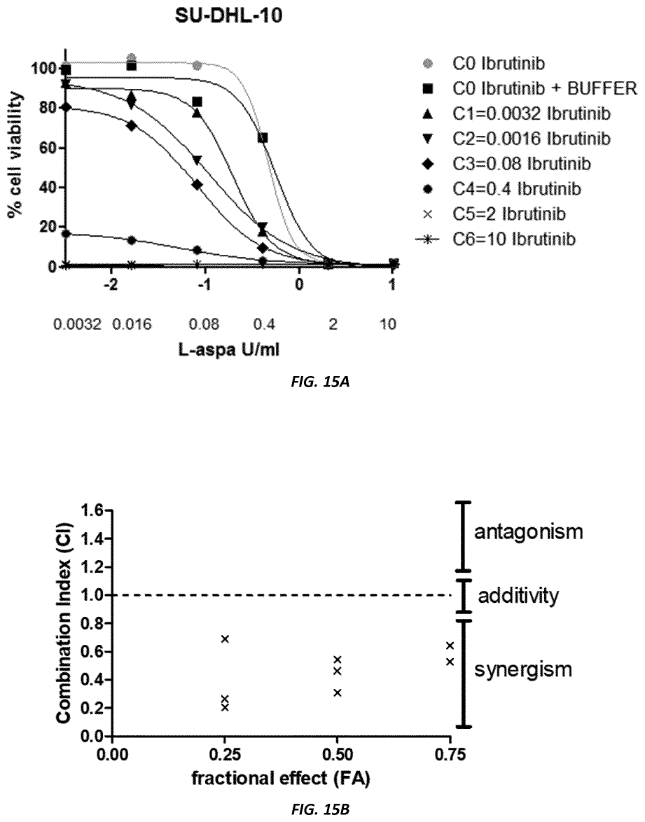

Furthermore, an ongoing study (performed substantially as described in Examples 4 & 6) indicates that the disclosed combinations may be synergistically effective against PDX cells taken from an extremely ASNase-refractory ALL patient. This particular patient's leukemia cells exhibited both IKZF1 deletion and PAX5 translocation, and, as indicated in FIGS. 14A-B, were significantly sensitive to the combination of native E. coli ASNase+Ibrutinib. These cells are currently amplifying in a mouse for subsequent analysis as detailed in Example 6.

Taken together, these data suggested to Applicants that it would be therapeutically useful to begin the combination treatment before the emergence of ASNase resistance. As such, in some embodiments, the invention encompasses a method for preventing ASNase resistance comprising the following steps: 1) determining the emergence of a predetermined level of ASNase blocking and/or inhibitory antibodies in a patient who is receiving ASNase therapy; 2) administering to the patient an effective amount of a BTKi to reduce and/or prevent the resistance; 3) continuing the ASNase therapy in combination with the BTKi therapy; 4) optionally discontinuing the BTKi therapy after the anti-ASNase antibodies reduced to some predetermined level; 5) optionally periodically monitoring the patient for the re-emergence of anti-ASNase antibodies; and 6) in the event of re-emergence of anti-ASNase antibodies at the predetermined level, repeat steps 2 to 5 as needed, for example, until the ALL has been eliminated.

Employing such a method would allow the clinician to selectively apply the disclose combination for ALL patients that would most benefit from the combination. In some embodiments, the method may be useful in cases where ALL patients relapse and are also intolerant to ASNase therapy. In such embodiments, the addition of the BTKi may allow for the re-sensitization of the relapsed patient's ALL cells to ASNase. In still further embodiments, these same methods and experiments may be extended to cancers outside of ALL, including to solid tumors and lymphomas.

### Example 10—Efficacy of AADAS+AADA Against Solid and Lymphoma Cancer Cells

To begin to determine the range of cancer types that are sensitive to the combination of AADAS+AADA, the components and their combinations were tested against 30 cell lines covering a variety of cancer cell types (Table 62). Two incubation times were tested. First, an overnight incubation detected early pro-apoptotic activity induced by the active ingredients (i.e. the AADAS and the AADA) via the measurement of caspase 3/7. Second, a longer incubation time (3-4 days) was performed to look simultaneously at viability, toxicity and apoptosis. For the experiments of this example, the AADA was the asparaginase known as SPECTRILA®, which is recombinantly produced E. coli ASNase having the amino acid sequence as set forth in NCBI Ref Seq WP_000394140.1, and having the ATC Code L01XX02. Ibrutinib was the AADAS as detailed above.

TABLE 62
Cancer cell lines

| Cell | Tissue/ | Cell | Tissue/ | Cell | Tissue/ |
|---|---|---|---|---|---|
| line | Type | line | Type | line | Type |
| Capan-1 | Pancreas | SU-DHL-8 | DLBCL | HepG2 | Liver |
| AsPC-1 | Pancreas | SU-DHL-10 | DLBCL | MDA- | Breast |
| | | | | MB-436 | |
| PANC-1 | Pancreas | Toledo | DLBCL | HCT-116 | Colon |
| Mia | Pancreas | NK-92 | NK | HT29 | Colon |
| PaCa-2 | | | Lymphoma | | |
| BxPC-3 | Pancreas | KHYG-1 | NK | SW620 | Colon |
| | | | Lymphoma | | |
| SW1990 | Pancreas | LN-229 | Glioblastoma | UM-UC-3 | Bladder |
| AGS | Stomach | U87 MG | Glioblastoma | RT-4 | Bladder |
| NCI-N87 | Stomach | PC-3 | Prostate | RS4; 11 | ALL |
| NIH: | Ovary | SK- | Liver | NALM-6 | ALL |
| OVCAR-3 | | HEP-1 | | | |
| SK-OV-3 | Ovary | Hep3B | Liver | SEM | ALL |

Cell culture conditions. Adherent tumor cells were grown as monolayer at 37° C. in a humidified atmosphere (5% CO₂, 95% air). For experimental use, tumor cells were rinsed twice with versene (ref.: 15040033, Thermofisher), detached from the culture flask by a 5-minute treatment with trypsin (ref.: 25300054, Thermofisher) and neutralized by addition of complete culture medium. The cells were counted and their viability assessed by 0.25% trypan blue exclusion assay.

TABLE 63
Culture conditions

| Cell line | Culture Medium | Growth |
|---|---|---|
| Capan-1 | IMDM + 20% FBS | Adherent |
| AsPC-1 | RPMI + 10% FBS + 1% GlutaMax + | Adherent |
| | 1% PS | |
| PANC-1 | DMEM + 10% FBS + 1% PS + 1% Na Pyr | Adherent |
| Mia | DMEM + 2.5% Horse serum + 10% FBS + | Adherent |
| PaCa-2 | 1% PS + 1% Na Pyr | |
| BxPC-3 | RPMI + 10% FBS + 1% GlutaMax + | Adherent |
| | 1% PS | |
| SW1990 | Leibovitz's + 10% FBS + 1% PS | Adherent |
| AGS | RPMI + 10% FBS + 1% GlutaMax | Adherent |
| NCI-N87 | RPMI + 10% FBS + 1% GlutaMax | Adherent |
| NIH: | RPMI + 10 mM Hepes + 1 mM Na | Adherent |
| OVCAR-3 | Pyr + 10% FBS + 2.5 g/l glucose + | |
| | 0.01 mg/ml bovine insulin | |
| SK-OV-3 | RPMI + 10% FBS + 1% GlutaMax | Adherent |
| SU-DHL-8 | RPMI 1640 + 10% FBS + 1% GlutaMax + | Suspension |
| | 1% PS | |
| SU-DHL-10 | RPMI 1640 + 10% FBS + 1% GlutaMax + | Suspension |
| | 1% PS | |
| Toledo | RPMI + 10 mM Hepes + 1 mM Na Pyr + | Suspension |
| | 15% FBS + 2.5 g/l glucose | |
| NK-92 | AlphaMEM + 10% FBS + 10% Horse | Suspension |
| | serum + 1% PS + 100 U/ml IL-2 + | |
| | 0.2 mM Myoinositol + 0.1 mM mercapto- | |
| | ethanol + 0.02 mM folic acid | |
| KHYG-1 | RPMI 1640 + 10% FBS + 1% GlutaMax + | Suspension |
| | 1% PS + 100 U/ml Il2 | |
| LN-229 | DMEM + 5% FBS + 1% Na Pyr | Adherent |
| U87 MG | EMEM + 10% FBS | Adherent |
| PC-3 | RPMI + 10% FBS + 1% GlutaMax | Adherent |
| SK-HEP-1 | RPMI 1640 + 10% FBS + 1% GlutaMax | Adherent |
| Hep3B2.1-7 | RPMI 1640 + 10% FBS + 1% GlutaMax | Adherent |
| HepG2 | EMEM + 10% FBS + 1 mM NaPyr + 0.1 | Adherent |
| | mM NEAA | |
| MDA- | RPMI 1640 + 10% FBS + 1% GlutaMax | Adherent |
| MB-436 | | |
| HCT-116 | McCoy's 5 A + 10% FBS | Adherent |
| HT29 | RPMI 1640 + 10% FBS + 1% GlutaMax | Adherent |
| SW620 | RPMI 1640 + 10% FBS + 1% GlutaMax | Adherent |
| UM-UC-3 | EMEM + 10% FBS + 1 mM NaPyr + 0.1 | Adherent |
| | mM NEAA | |
| RT-4 | McCoy's 5 A + 10% FBS | Adherent |
| RS4; 11 | RPMI + 10% FBS + 1% PS | Suspension |
| NALM-6 | RPMI + 10% FBS + 1% PS | Suspension |
| SEM | RPMI + 10% FBS + 1% PS | Suspension |

Cell plating for 3 to 4 day incubation. The tumor cells were seeded at optimal density (Table 64) in 96-well flat-bottom microtitration plates. Cells were seeded in 90 μL of drug free minimal essential medium and incubated at 37° C. under 5% CO₂. Adherent cells were seeded overnight before treatment whereas cells in suspension were seeded on the same day as treatment.

TABLE 64
Seeding conditions

| Cell | Cells/ | Cell | Tissue/ | Cell | Tissue/ |
|---|---|---|---|---|---|
| line | Well | line | Type | line | Type |
| Capan-1 | 5000 | SU-DHL-8 | 10000 | HepG2 | 1000 |
| AsPC-1 | 2500 | SU-DHL-10 | 5000 | MDA- | 2500 |
| | | | | MB-436 | |
| PANC-1 | 5000 | Toledo | 25000 | HCT-116 | 500 |
| Mia PaCa-2 | 2000 | NK-92 | 7500 | HT29 | 500 |
| BxPC-3 | 1500 | KHYG-1 | 2500 | SW620 | 750 |
| SW1990 | 7500 | LN-229 | 1000 | UM-UC-3 | 1000 |
| AGS | 1000 | U87 MG | 1000 | RT-4 | 2500 |
| NCI-N87 | 2500 | PC-3 | 1000 | RS4; 11 | 100000 |
| NIH: | 2000 | SK-HEP-1 | 2000 | NALM-6 | 100000 |
| OVCAR-3 | | | | | |
| SK-OV-3 | 2000 | Hep3B2.1-7 | 2500 | SEM | 100000 |

Tumor cell plating for overnight incubation. The tumor cells were seeded at optimal density (Table 65) in 96-well flat-bottom microtitration plates. Cells were seeded in 90 μL of drug free minimal essential medium and incubated at 37° C. under 5% CO₂. Adherent cells were seeded overnight before treatment whereas cells in suspension were seeded on the day of treatment.

TABLE 65
Seeding conditions

| Cell | Cells/ | Cell | Tissue/ | Cell | Tissue/ |
|---|---|---|---|---|---|
| line | Well | line | Type | line | Type |
| Capan-1 | 15000 | SU-DHL-8 | 30000 | HepG2 | 3000 |
| AsPC-1 | 7500 | SU-DHL-10 | 15000 | MDA- | 7500 |
| | | | | MB-436 | |
| PANC-1 | 15000 | Toledo | 75000 | HCT-116 | 1500 |
| Mia | 6000 | NK-92 | 22500 | HT29 | 1500 |
| PaCa-2 | | | | | |
| BxPC-3 | 4500 | KHYG-1 | 7500 | SW620 | 2250 |
| SW1990 | 22500 | LN-229 | 3000 | UM-UC-3 | 3000 |
| AGS | 3000 | U87 MG | 3000 | RT-4 | 7500 |
| NCI-N87 | 7500 | PC-3 | 3000 | RS4; 11 | |
| NIH: | 6000 | SK-HEP-1 | 6000 | NALM-6 | |
| OVCAR-3 | | | | | |
| SK-OV-3 | 6000 | Hep3B2.1-7 | 7500 | SEM | 250000 |

Compounds were added to the plates containing cells the day following plating. Each compound was tested alone at 6 doses. For Ibrutinib, the doses were 0.0032, 0.016, 0.08, 0.4, 2, and 10 μM. These doses were prepared from a stock solution of 10 mM in 100% DMSO, with successive 5-fold dilutions in 100% DMSO to provide the stock solutions for the lower concentrations. Ibrutinib was diluted 100-fold from stock solution in culture medium and 10 μL were added to the cells. The final concentration of DMSO was 0.1%, and 10 μL of DMSO 0.1% was used as the negative control. For Asparaginase, the doses tested were 0.0032, 0.016, 0.08, 0.4, 2, and 10 U/ml. These were prepared as stock solutions of 10,000 U/ml in DPBS. Successive 5-fold dilutions in DPBS provided the stock solutions for the lower concentrations. Asparaginase was diluted 100-fold from stock solution in culture medium and 10 μL was added to the cells. The final concentration of DPBS was 0.1%, and 10 μL of DPBS 0.1% was used as the negative control.

The two active ingredients were also tested in combinations at 6×6 doses, with the primary dilutions prepared as above. Ibrutinib was diluted 50-fold from stock solution in culture medium and 5 μL were added to the cells. The final concentration of DMSO were 0.1%, and 5 μL of DMSO 0.1% was used as negative control. Asparaginase was diluted 50-fold from stock solution in culture medium and 5 μL were added to the cells. The final concentration of DPBS was 0.1%, and 5 μL of DPBS 0.1% were used as negative control. Experiments were performed in duplicate.

TABLE 66
Plate map

| | 1 | 2 | 3 | 4 | 5 | 6 | 7 | 8 | 9 | 10 | 11 | 12 |
|---|---|---|---|---|---|---|---|---|---|---|---|---|
| A | CM | CM | CM | CM | CM | CM | CM | CM | CM | CM | CM | CM |
| B | CM | A0-I0 | A1-10 | A2-I0 | A3-I0 | A4-I0 | A5-I0 | A6-I0 | B-A | B-I | B-1 | CM |
| C | CM | A0-I1 | A1-I1 | A2-I1 | A3-I1 | A4-I1 | A5-I1 | A6-I1 | A1 | I1 | B-A | CM |
| D | CM | A0-I2 | A1-I2 | A2-I2 | A3-I2 | A4-I2 | A5-I2 | A6-I2 | A2 | I2 | A6I6 | CM |
| E | CM | A0-I3 | A1-I3 | A2-I3 | A3-I3 | A4-I3 | A5-I3 | A6-I3 | A3 | I3 | A6I6 | CM |
| F | CM | A0-I4 | A1-I4 | A2-I4 | A3-I4 | A4-I4 | A5-I4 | A6-I4 | A4 | I4 | B-AI | B-AI |
| G | CM | A0-I5 | A1-I5 | A2-I5 | A3-I5 | A4-I5 | A5-I5 | A6-I5 | A5 | I5 | B-AI | B-AI |
| H | CM | A0-I6 | A1-I6 | A2-I6 | A3-I6 | A4-I6 | A5-I6 | A6-I6 | A6 | I6 | w/o cell | w/o cell |

CM = culture media
ASNase concentrations (U/ml) (diluted in DPBS)

| | | | | | | |
|---|---|---|---|---|---|---|
| A0 | A1 | A2 | A3 | A4 | A5 | A6 |
| 0 | 0.0032 | 0.016 | 0.08 | 0.4 | 2 | 10 |

Ibrutinib concentrations (μM) (diluted in DMSO)

| | | | | | | |
|---|---|---|---|---|---|---|
| I0 | I1 | I2 | I3 | I4 | I5 | I6 |
| 0 | 0.0032 | 0.016 | 0.08 | 0.4 | 2 | 10 |

| | |
|---|---|
| B-A: | DPBS only; negative control for ASNase (background) |
| A1-A6: | ASNase only; positive control for ASNase (with DPBS diluent) |
| B-I: | DMSO only; negative control for ibrutinib (background) |
| I1-I6: | Ibrutinib only positive control for ibrutinib (with DMSO diluent) |
| B-AI: | PBS + DMSO; negative control for ASNase + ibrutinib (background) |
| A6-I6 | ASNase + Ibrutinib; + control for ASNase + ibrutinib (with DPBS + DMSO diluents) |

Two protocols of incubation were performed: overnight and over 3/4 days as indicated.

TABLE 67
Seeding conditions

| | Incu- | | Incu- | | Incu- |
|---|---|---|---|---|---|
| Cell | bation | Cell | bation | Cell | bation |
| line | (days) | line | (days) | line | (days) |
| Capan-1 | 4 | SU-DHL-8 | 4 | HepG2 | 3 |
| AsPC-1 | 4 | SU-DHL-10 | 4 | MDA- | 4 |
| | | | | MB-436 | |
| PANC-1 | 4 | Toledo | 4 | HCT-116 | 4 |
| Mia PaCa-2 | 3 | NK-92 | 3 | HT29 | 4 |
| BxPC-3 | 4 | KHYG-1 | 4 | SW620 | 4 |
| SW1990 | 4 | LN-229 | 4 | UM-UC-3 | 4 |
| AGS | 4 | U87 MG | 4 | RT-4 | 4 |
| NCI-N87 | 4 | PC-3 | 4 | RS4; 11 | 3 |
| NIH: | 4 | SK-HEP-1 | 3 | NALM-6 | 3 |
| OVCAR-3 | | | | | |
| SK-OV-3 | 4 | Hep3B2.1-7 | 4 | SEM | 3 |

Active ingredient efficacy was measured after an overnight incubation time using the Caspase-Glo 3/7™ Assay (Promega). The assay provides a proluminescent caspase-3/7 substrate, which contains the tetrapeptide sequence DEVD. This substrate is cleaved to release aminoluciferin, a substrate of luciferase used in the production of light. The assay was performed as recommended by the manufacturer. Briefly, the plate was read using EnVision plate reader (PerkinElmer). Compound efficacy after 3-4 days of incubation was monitored using the ApoTox-Glo™ Triplex Assay (Promega). This assay combines three assay chemistries to assess viability, cytotoxicity and apoptosis events in the same cell-based assay well. First, viability and cytotoxicity are determined by measuring two differential protease biomarkers simultaneously with the addition of a single non-lytic reagent containing two peptide substrates. A second reagent containing luminogenic DEVD-peptide substrate for caspase-3/7 and Ultra-Glo™ Recombinant Thermostable Luciferase is added. Caspase-3/7 cleavage of the substrate releases luciferin, which is a substrate for luciferase and generates light. The light output, measured with a luminometer, correlates with caspase-3/7 activation as a key indicator of apoptosis. The assay was performed as recommended by the manufacturer. Briefly, the plate was read using EnVision plate reader (PerkinElmer). Viability was measured in fluorescence units at wavelengths Ex 400/Em 505. Cytotoxicity was monitored in fluorescence units at wavelengths Ex 485/Em 520. Apoptosis was measured in luminescence.

RESULTS. The majority of the cell lines demonstrated greater sensitivity to the combination of ASNase and Ibrutinib than they did to either active ingredient alone (i.e. additivity and synergism). In some cases, there is strong evidence of synergistic/supra-additive efficacy. In other cases, the cells were not responsive to Ibrutinib, but were significantly more responsive to the combination than they were to ASNase alone (e.g. Bladder, Glioblastoma and Hepatic cancer cells). In still other cases, the cells were somewhat resistant to ASNase, but that resistance was at least partially overcome by the addition of Ibrutinib.

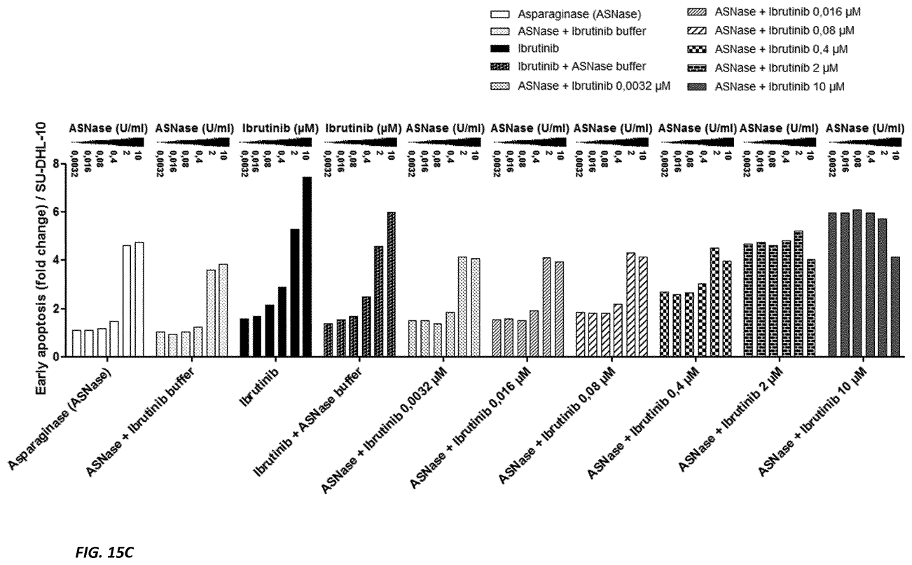

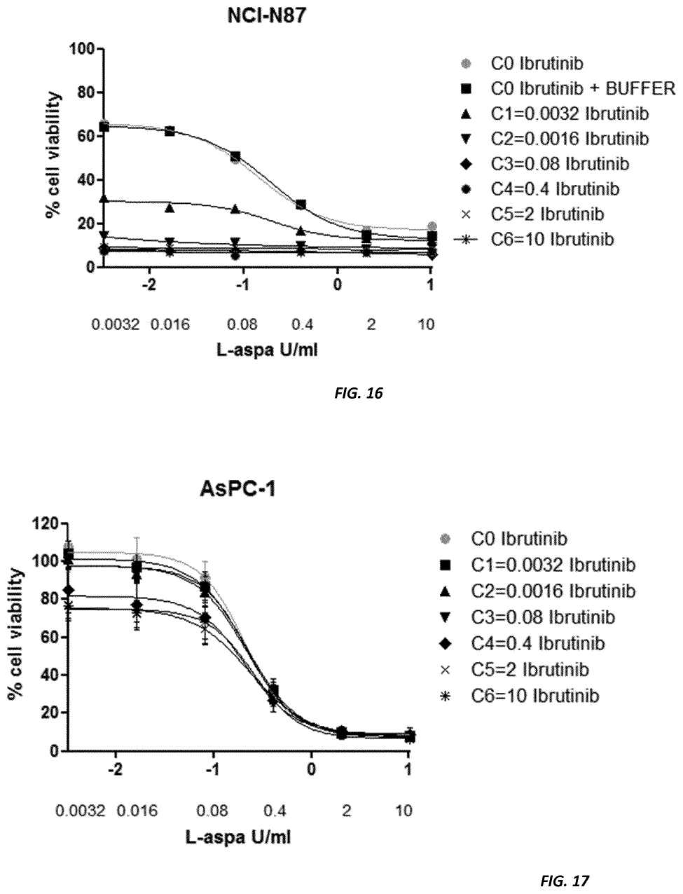

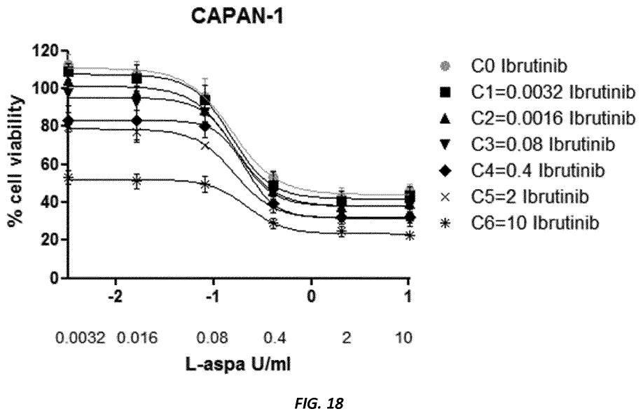

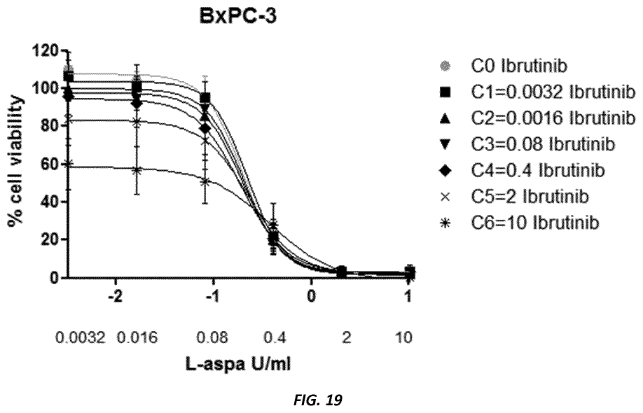

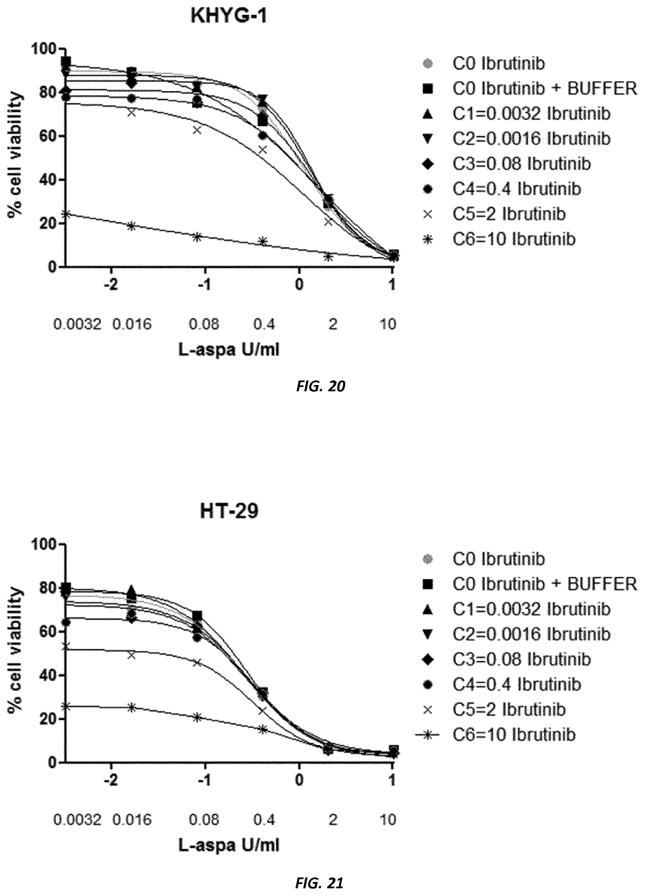

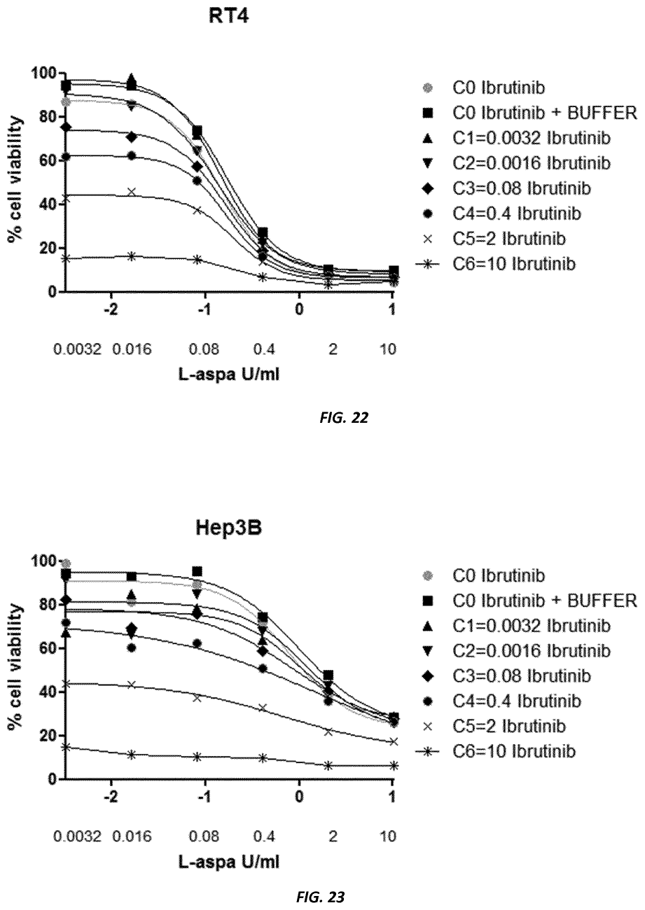

FIGS. 15 to 23 present % cell viability curves for SU-DHL-10 (DLBCL), NCI-N87 (gastric cancer), AsPC-1/CAPAN-1/BxPC-3 (pancreatic cancer), KHYG-1 (NK Lymphoma), HT-29 (Colorectal cancer), RT4 (bladder cancer) and Hep3B (hepatic cancer) cells. And as indicted in FIGS. 15B-C, the combination of ASNase and Ibrutinib were highly synergistically effective against DLBCL cells. And while NCI-N87 Gastric cancer cells were quite sensitive to each active ingredient alone, they were synergistically sensitive to the combination (FIG. 16).

Taken together, the foregoing results support the broad use of synergistic combinations of amino acid depletion agents (AADA) (e.g. ASNase) and amino acid depletion agent sensitizers (AADAS) (e.g. BTKi) to kill cancer cells and to treat patients suffering from liquid cancers, solid cancers and lymphomas.

The invention will now be described in the following non-limiting, numbered embodiments.

1. A pharmaceutical composition, kit or fixed-dose combination comprising:

(a) an effective amount of an amino acid depletion agent (AADA) selected from an asparaginase (ASNase), an arginase (ARGase), an arginine deiminase (ADI), a methioninase/methioninase (METase), an adenosine deaminase (ADA), an IDO, a TDO, fumagillin, a diet low in a selected amino acid, and a glutaminase (GLNase); and

(b) an effective amount of an amino acid depletion agent sensitizer (AADAS), wherein the AADAS is a Bruton's Tyrosine Kinase inhibitor (BTKi);

for use in the treatment of a of disease or condition in a subject or patient in need of treatment thereof, wherein the disease or condition is not effectively treated by either the ADAA or the AADAS alone, or wherein the amounts of the AADA and the AADAS are synergistically effective in treating the disease or condition, or wherein the amount of the AADAS is sufficient to sensitize AADA-resistant cells to AADA, or wherein the amount of the AADAS is sufficient to enable the use of a smaller amount of AADA to treat a disease or condition wherein an effective amount of the AADA would produce unacceptable toxicity in the subject or patient.

2. The pharmaceutical combination of embodiment 1, wherein the AADA is ASNase and the AADAS is a small molecule BTKi, and wherein the AADA and AADAS are separate entities, delivered sequentially or simultaneously, and are present in synergistically therapeutically effective amounts.

3. The pharmaceutical combination of embodiment 1 or 2, wherein the BTKi is selected from ibrutinib, acalabrutinib, zanabrutinib, tirabrutinib, M7583, vecabrutinib, CT-1530, ARQ 531, DTRMWXHS-12, TG-1701, spebrutinib, CC-292, CG′806, evorbrutinib, RG7845, GDC-0853, poseltinib, LY3337641, HM71224, PRN1008, BMS-986142, PRN2246, TAK-020, AC0058, BUB-068, a BTKi having substantially the same in vivo PK/PD profile and mechanism of action as any of the foregoing and combinations thereof.

4. The pharmaceutical combination of any one of embodiments 1 to 3, wherein the ASNase is selected from a native E. coli asparaginase (e.g. ELSPAR, Lundbeck Inc.), an E. coli-derived peg-conjugated ASNase (e.g. ONCASPAR®, Servier), an E. chrysanthemi ASNase (e.g. ERWINAZE®, EUSA Pharma), a human-derived ASNase, an ASNase having substantially the same in vivo PK/PD profile as any of the foregoing and combinations thereof.

5. A method of treating cancer, comprising administering to a subject in need thereof synergistically effective amounts of an ASNase and a BTKi.

6. The method of embodiment 5, wherein the amount of the ASNase would be subtherapeutic for the subject if it were not administered sequentially or simultaneously as a combination therapy with the BTKi.

7. The method of embodiment 5 or 6, wherein the amount of the BTKi would be subtherapeutic for the subject's cancer were it not administered sequentially or simultaneously as a combination therapy with the ASNase.

8. The method of any one of claims 5 to 7, wherein the cancer is acute lymphoblastic leukemia (ALL), acute myeloid leukemia (AML), DLBCL, Gastric cancer, Pancreatic cancer, NK Lymphoma, Colorectal cancer, Bladder cancer or Hepatic cancer.

9. The method of any one of embodiments 5 to 8, wherein the cancer is resistant to ASNase treatment.

10. The method of any one of embodiments 5 to 9, wherein the ASNase and the BTKi are sequentially administered, preferably wherein the BTKi is administered before the ASNase.

11. The method of any one of embodiments 5 to 10, wherein the cancer comprises a cancer-initiating stem cell.

12. The method any one of embodiments 5 to 11, wherein the cancer comprises tumor cells that are resistant to ASNase-induced apoptosis or cell death.

13. The method of any one of embodiments 5 to 7, wherein the cancer comprises a blood-borne, brain, pancreatic, cervical, lung, head and neck, breast or gastro-intestinal cancer.

14. The method of embodiment 13, wherein the cancer comprises a DLBCL or other B cell lymphoma, a pancreatic cancer, a colorectal cancer, a gastric cancer or a triple negative breast cancer.

15. The method of embodiment 14, wherein the cancer comprises a DLBCL or another lymphoma.

16. The method of any one of embodiments 5 to 15, wherein the ASNase and/or the BTKi are administered by injection or wherein the ASNase is administered by injection and the BTKi is administered orally.

17. The method of any one of embodiments 5 to 16, wherein the ASNase is selected from a native E. coli asparaginase (e.g. ELSPAR, Lundbeck Inc.), an E. coli-derived peg-conjugated ASNase (e.g. ONCASPAR®, Servier), an E. chrysanthemi ASNase (e.g. ERWINAZE®, EUSA Pharma), and a human-derived ASNase.

18. The method of any one of embodiments 5 to 17, wherein the ASNase and the BTKi are separate entities.

19. The method of any one of embodiments 5 to 18, wherein the BTKi is a covalent, irreversible BTKi or is a safe and effective agent capable of knocking down or eliminating BTK activity in the cancer cells.

20. The method of any one of embodiments 5 to 19, wherein the ASNase is encapsulated in red blood cells (RBCs) and the BTKi is co-formulated with said encapsulated RBCs.

21. A pharmaceutical composition, kit or fixed dose combination for use in treatment of cancer in subject in need of treatment therefor, comprising a pharmaceutically acceptable carrier and a combination of a BTKi and an ASNase, wherein the combination contains a subtherapeutic dose of the BTKi and a subtherapeutic dose of the ASNase, and neither the dose of the BTKi nor the dose of the ASNase are or would be sufficient alone to treat the cancer.

22. The composition for the use of embodiment 21, comprising at least one dose of the BTKi and at least one dose of the ASNase.

23. The composition for the use of embodiment 21 or 22, comprising from about 0.05 mg to about 1.0 mg of the BTKi and from about 50 to about 500 U of the ASNase.

24. The composition for the use of any one of embodiments 21 to 23, wherein the dose of the BTKi is from about 5 to about 50 mg/kg bodyweight of the subject and the dose of the ASNase is about 50 to about 500 IU/kg bodyweight of the subject.

25. The composition for the use of any one of embodiments 21 to 24, wherein the dose of the BTKi is from about 10 to about 40 mg/kg and the dose of the ASNase is about 100 to about 400 IU/kg.

26. The composition for the use of any one of embodiments 21 to 25, wherein the dose of the BTKi is from about 15 to about 35 mg/kg and the dose of the ASNase is about 200 to about 400 IU/kg.

27. The composition for the use of any one of embodiments 21 to 26, wherein the dose of the BTKi is from about 25 mg/kg and the dose of the ASNase is about 300 IU/kg.

28. The composition for the use of any one of embodiments 21 to 27, wherein the BTKi is ibrutinib and the ASNase is either a PEG-ASNase or an RBC-encapsulated ASNase.

29. The composition for the use of any one of embodiment 21 to 28, comprising from about 10 to about 35 mg/kg ibrutinib, preferably dissolved in 5% DMSO+30% PEG 300+5% Tween 80+ddH2O; and about 200 to 500 IU/kg PEG-ASNase or RBC-encapsulated ASNase.

30. A pharmaceutical combination comprising (i) a BTKi and (ii) an AADA, or a pharmaceutically acceptable salt thereof, respectively, or a prodrug thereof, respectively, and at least one pharmaceutically acceptable carrier.

31. The pharmaceutical combination according to embodiment 30 for simultaneous, separate or sequential use of the components (i) and (ii).

32. The pharmaceutical combination according to embodiment 30 or 31 in the form of a fixed combination.

33. The pharmaceutical combination according to any one of embodiments 30 to 32 in the form or a kit of parts for the combined administration where the BTKi and the AADA may be administered independently at the same time or separately within time intervals, especially where these time intervals allow that the combination partners are jointly active.

34. The pharmaceutical combination according to any one of embodiments 30 to 33, wherein the BTKi is ibrutinib, acalabrutinib, zanabrutinib, tirabrutinib, M7583, vecabrutinib, CT-1530, ARQ 531, DTRMWXHS-12, TG-1701, spebrutinib, CC-292, CG′806, evorbrutinib, RG7845, GDC-0853, poseltinib, LY3337641, HM71224, PRN1008, BMS-986142, PRN2246, TAK-020, AC0058, BIIB-068, a BTKi having substantially the same in vivo PK/PD profile and mechanism of action as any of the foregoing, or combinations thereof; and wherein the amino acid depletion agent is ASNase; or a pharmaceutically acceptable salt or prodrug thereof, respectively.

35. The pharmaceutical combination according to any one of embodiments 30 to 34, wherein the ASNase is selected from a native E. coli asparaginase (e.g. ELSPAR, Lundbeck Inc.), an E. coli-derived peg-conjugated ASNase (e.g. ONCASPAR®, Servier), an E. chrysanthemi ASNase (e.g. ERWINAZE®, EUSA Pharma), and a human-derived ASNase.

35. The pharmaceutical combination according to any one of embodiments 30 to 35, further comprising a co-agent, or a pharmaceutically acceptable salt or a prodrug thereof.

36. The pharmaceutical combination according to any one of embodiments 30 to 35 in the form of a co-formulated combination product.

37. Use of the pharmaceutical combination or combination product according to any one of embodiments 30 to 36 for treating cancer that is or has become resistant to treatment with either the BTKi or the ASNase.

38. A combination of (i) a BTKi and (ii) an amino acid depletion agent, for the manufacture of a medicament or a pharmaceutical product, especially a combination or combination product according to embodiment 30, for treating cancer.

39. A pharmaceutical product or a commercial package comprising a combination or combination product according to embodiment 30, in particular together with instructions for simultaneous, separate or sequential use thereof in the treatment of a BTKi and an AADA for the treatment of cancer.

40. A pharmaceutical combination according to embodiment 30, for use in the treatment of cancer or as a medicine.

41. A method of inducing apoptosis in a tumor cell in vivo in a mammalian subject, wherein the tumor cell is resistant to treatment with an AADA, or the tumor cell that has only been rendered quiescent by said AADA, comprising administering an effective amount of an AADAS, administering said AADA, and allowing sufficient time for the tumor cells to undergo apoptosis, thereby inducing the apoptosis in the tumor cell.

42. The method of embodiment 41, wherein the AADAS is administered before the AADA.

43. The method of embodiment 41 or 42, wherein the AADAS is administered 1, 2, 3, 4, 5 or more days prior to the administration of the AADA.

44. The method of any one of embodiments 41 to 43, wherein the AADAS is administered in an amount from about 5 to about 50 mg/kg bodyweight of the subject.

45. The method of any one of embodiments 41 to 44, wherein the AADAS is administered in an amount from about 10 to about 40 mg/kg.

46. The method of any one of embodiments 41 to 45, wherein the AADAS is administered in an amount from about 20 to about 30 mg/kg.

47. The method of any one of embodiments 41 to 46, wherein the AADAS is a BTKi and the AADA is an ASNase.

48. The method of any one of embodiments 40 to 47, wherein the BTKi is administered in an amount from about 15 to about 35 mg/kg and the ASNase is administered in an amount from about 200 to about 400 IU/kg.

49. The method of any one of embodiments 40 to 48, wherein the BTKi is administered in an amount from about 20 to about 30 mg/kg and the ASNase is administered in an amount from about 200 to about 400 IU/kg.

50. The method of any one of embodiments 40 to 49, wherein the BTKi is ibrutinib and the ASNase is encapsulated in enucleated RBCs.

51. A method of treating a subject or patient suffering from cancer and previously unsuccessfully treated with ASNase, wherein the subject or patient exhibited hypersensitivity to and/or silent inactivation of said ASNase, comprising administering a sensitizing-effective amount of an AADAS and an apoptosis-inducing effective amount of an AADA.

52. The method of embodiment 51, comprising the step of administering an effective amount of the composition of any one of embodiments 1-4 or any one of embodiments 21-40.

53. The method of embodiment 51 or 52, wherein the AADAS is administered in an amount from about 5 to about 50 mg/kg bodyweight of the subject.

54. The method of embodiment 53, wherein the AADAS is administered in an amount from about 10 to about 40 mg/kg.

55. The method of embodiment 54, wherein the AADAS is administered in an amount from about 20 to about 30 mg/kg.

56. The method of embodiment 55, wherein the AADAS is a BTKi and the AADA is an ASNase.

57. The method of embodiment 56, wherein the BTKi is administered in an amount from about 15 to about 35 mg/kg and the ASNase is administered in an amount from about 200 to about 400 IU/kg.

58. The method of embodiment 57, wherein the BTKi is administered in an amount from about 20 to about 30 mg/kg and the ASNase is administered in an amount from about 200 to about 400 IU/kg.

59. The method of any one of embodiments 51 to 58, wherein the BTKi is ibrutinib and the ASNase is encapsulated in enucleated RBCs.

60. The method of any one of embodiments 56 to 59, wherein the BTKi and the ASNase are administered to the subject or patient in amounts that, if give separately, would not induce apoptosis in a majority of the cancer cells.

61. The method of any one of the preceding embodiments, wherein the AADA is a diet low in methionine or asparagine.

62. The method of embodiment 61, wherein the AADA is a diet low in methionine and asparagine.

63. The method of embodiment 61, wherein the low methionine diet is begun about 7 days after the administration of the AADAS.

64. The method of embodiment 61, wherein the low asparagine diet is begun about 7 days after the administration of the AADAS.

65. The method of embodiment 62, wherein the low methionine and low asparagine diet is begun about 7 days after the administration of the AADAS.

66. The composition or method of any one of the preceding embodiments comprising or making use of no traditional chemotherapeutic drug.

67. A method for preventing ASNase resistance (silent inactivation) comprising the following steps:

1) determining the emergence of a predetermined level of ASNase blocking and/or inhibitory anti-ASNase antibodies in a patient who is receiving ASNase therapy, said level being indicative of an early phase in the development of resistance against ASNase;

2) administering to the patient an effective amount of a BTKi to reduce and/or prevent the levels of blocking and/or inhibitory anti-ASNase antibodies past a predetermined level; and

3) continuing to administer effective amounts of ASNase in combination with a resistance-blocking effective amount of BTKi.

68. The method of embodiment 67, comprising the steps of discontinuing the administration of the BTKi when the anti-ASNase blocking and/or inhibitory antibody levels have reduced to below the initial predetermined level of step 1; periodically monitoring the patient for the re-emergence of anti-ASNase antibody levels in excess of the predetermined level; and, in the event of re-emergence of anti-ASNase antibodies at or above the predetermined level, repeating steps 2 to 5 as needed, until the ALL is in complete remission.

69. A method of reversing ASNase resistance in a patient suffering from a cancer that has ceased to be sensitive to ASNase therapy as a result of antibody induced inactivation, wherein the cancer is optionally a leukemia, a lymphoma or a solid cancer, comprising administering to the patient an effective amount of a BTKi to re-sensitize the patient's cancer to ASNase therapy, thereby reversing the resistance.

70. The method of any one of embodiments 67 to 69, wherein the BTKi is administered in an amount from about 5 to about 50 mg/kg, about 10 to about 40 mg/kg, or about 20 to about 30 mg/kg bodyweight of the patient.

71. The method of any one of embodiments 67 to 70, wherein the amount of BTKi is sufficient to attenuate or completely inhibit ASNase-induced increases in ASNS expression.

72. The method of embodiment 71, wherein BTKi inhibits ASNase-induced ASNS expression by attenuating or inhibiting ASNase-induced increases in ATF4 expression.

73. A method of blocking an in culture or in vivo cell from mounting an effective amino acid deprivation stress response (AADSR) comprising treating the cell, or administering to a patient or subject comprising the cell, an effective amount of an AADA and an effective amount of an AADAS, thereby blocking the cell from mounting the effective AADSR; optionally wherein the AADAS is a BTKi and the AADA is an ASNase.

74. The method of embodiment 73, wherein the amount of ASNase would be sufficient to induce GCN2-mediated, ATF4-mediated increases in ASNS expression, in absence of the BTKi.

75. The method of any of the foregoing embodiments, wherein the amount of BTKi administered to the patient or subject is sufficient to reverse ASNase hypersensitivity, wherein the hypersensitivity is optionally selected from acute allergic reactions to ASNase ranging from mild symptoms to systemic anaphylaxis.

The invention will now be described in the following non-limited claims.

1. A pharmaceutical composition, kit or fixed-dose combination comprising:
(a) an effective amount of an amino acid depletion agent (AADA) selected from an asparaginase (ASNase), an arginase (ARGase), an arginine deiminase (ADI), a methionase (METase), an adenosine deaminase (ADA), an IDO, a TDO, fumagillin, a diet low in a selected amino acid, and a glutaminase (GLNase); and
(b) an effective amount of an amino acid depletion agent sensitizer (AADAS), wherein the AADAS is a Bruton's Tyrosine Kinase inhibitor (BTKi);
for use in the treatment of a of disease or condition in a subject or patient in need of treatment thereof, wherein the disease or condition is not effectively treated by either the ADAA or the AADAS alone, or wherein the amounts of the AADA and the AADAS are synergistically effective in treating the disease or condition, or wherein the amount of the AADAS is sufficient to sensitize AADA-resistant cells to AADA, or wherein the amount of the AADAS is sufficient to enable the use of a smaller amount of AADA to treat a disease or condition wherein an effective amount of the AADA would produce unacceptable toxicity in the subject or patient.

2. The pharmaceutical combination of claim 1, wherein the AADA is ASNase and the AADAS is a small molecule BTKi, and wherein the AADA and AADAS are separate entities, delivered sequentially or simultaneously, and are present in synergistically therapeutically effective amounts.

3. The pharmaceutical combination of claim 1, wherein the BTKi is selected from ibrutinib, acalabrutinib, zanabrutinib, tirabrutinib, M7583, vecabrutinib, CT-1530, ARQ 531, DTRMWXHS-12, TG-1701, spebrutinib, CC-292, CG′806, evorbrutinib, RG7845, GDC-0853, poseltinib, LY3337641, HM71224, PRN1008, BMS-986142, PRN2246, TAK-020, AC0058, BIIB-068, a BTKi having substantially the same in vivo PK/PD profile and mechanism of action as any of the foregoing and combinations thereof.

4. The pharmaceutical combination of claim 1, wherein the ASNase is selected from a native E. coli asparaginase (e.g. ELSPAR, Lundbeck Inc.), an E. coli-derived peg-conjugated ASNase (e.g. ONCASPAR®, Servier), an E. chrysanthemi ASNase (e.g. ERWINAZE®, EUSA Pharma), a human-derived ASNase, an ASNase having substantially the same in vivo PK/PD profile as any of the foregoing and combinations thereof.

5. A method of treating cancer, comprising administering to a subject in need thereof synergistically effective amounts of an ASNase and a BTKi.

6. The method of claim 5, wherein the amount of the ASNase would be subtherapeutic for the subject if it were not administered sequentially or simultaneously as a combination therapy with the BTKi.

7. The method of claim 5 or 6, wherein the amount of the BTKi would be subtherapeutic for the subject's cancer were it not administered sequentially or simultaneously as a combination therapy with the ASNase.

8. The method of claim 5, wherein the cancer is acute lymphoblastic leukemia (ALL), acute myeloid leukemia (AML), DLBCL, Gastric cancer, Pancreatic cancer, NK Lymphoma, Colorectal cancer, Bladder cancer, Hepatic cancer or a Glioma or Glioblastoma.

9. The method of claim 5, wherein the cancer is resistant to ASNase treatment.

10. The method of claim 5, wherein the ASNase and the BTKi are sequentially administered, preferably wherein the BTKi is administered before the ASNase.

11. The method of claim 5, wherein the cancer comprises a cancer-initiating stem cell.

12. The method of claim 5, wherein the cancer comprises tumor cells that are resistant to ASNase-induced cell death optionally selected from apoptosis.

13. The method of claim 5, wherein the cancer comprises a blood-borne, brain, pancreatic, cervical, lung, head and neck, breast or gastro-intestinal cancer.

14. The method of claim 13, wherein the cancer comprises a DLBCL or other B cell lymphoma, a pancreatic cancer, a colorectal cancer, a gastric cancer or a triple negative breast cancer.

15. The method of claim 14, wherein the cancer comprises a DLBCL or another lymphoma.

16. The method of claim 5, wherein the ASNase and/or the BTKi are administered by injection or wherein the ASNase is administered by injection and the BTKi is administered orally.

17. The method of claim 5, wherein the ASNase is selected from a native E. coli asparaginase (e.g. ELSPAR, Lundbeck Inc.), an E. coli-derived peg-conjugated ASNase (e.g. ONCASPAR®, Servier), an E. chrysanthemi ASNase (e.g. ERWINAZE®, EUSA Pharma), and a human-derived ASNase.

18. The method of claim 5, wherein the ASNase and the BTKi are separate entities.

19. The method of claim 5, wherein the BTKi is a covalent, irreversible BTKi or is a safe and effective agent capable of knocking down or eliminating BTK activity in the cancer cells.

20. The method of claim 5, wherein the ASNase is encapsulated in red blood cells (RBCs) and the BTKi is co-formulated with said encapsulated RBCs.
